(19) United States
(12) Patent Application Publication
(10) Pub. No.: US 2026/0337586 A1
(43) Pub. Date: Oct. 29, 2026

(54) ENGINEERED DNA POLYMERASE VARIANTS

(71) Applicant: Codexis, Inc., Redwood City, CA (US)

(72) Inventors: Jason Fell, Martinez, CA (US); Sandy M. Gomes, Redwood City, CA (US); Supriya Vijaykumar Kadam, San Jose, CA (US); Anders Matthew Knight, Mountain View, CA (US); Larson Lyle Matzdorff, San Francisco, CA (US); Mathew G. Miller, San Carlos, CA (US); Zhe Rui, Newark, CA (US)

(21) Appl. No.: 19/111,260

(22) PCT Filed: Sep. 12, 2023
(86) PCT No.: PCT/US2023/073997

§ 371 Date: Mar. 12, 2025

Related U.S. Application Data
(60) Provisional application No. 63/375,371, filed on Sep. 12, 2022.
(60) Provisional application No. 63/494,211, filed on Apr. 4, 2023.

(51) Int. Cl. C12N 9/12; C12P 19/34; C12Q 1/6806; C12Q 1/6844

(52) U.S. Cl. CPC C12N 9/1252; C12P 19/34; C12Q 1/6806; C12Q 1/6844; C12Y 207/07007

(57) ABSTRACT

The present disclosure relates to engineered DNA polymerase polypeptides and compositions thereof, as well as polynucleotides encoding the engineered DNA polymerase polypeptides. The present disclosure also provides methods of using the engineered DNA polymerase polypeptides or compositions thereof for diagnostic and other purposes.

### CROSS-REFERENCE TO RELATED APPLICATIONS

This application claims the benefit of U.S. Provisional Application No. 63/375,371, filed Sep. 12, 2022, and U.S. Provisional Application No. 63/494,211, filed Apr. 4, 2023, the contents of which are incorporated by reference herein.

### REFERENCE TO SEQUENCE LISTING, TABLE OR COMPUTER PROGRAM

The Sequence Listing concurrently submitted herewith as file name CX9-226WO2_ST26.xml, created on Sep. 12, 2023, with a file size of 1,322,734 bytes, is part of the specification and is incorporated by reference herein in its entirety.

### TECHNICAL FIELD

The present disclosure provides engineered DNA polymerase polypeptides and compositions thereof, as well as polynucleotides encoding the engineered DNA polymerase polypeptides. The disclosure also provides methods of using the engineered DNA polymerase or compositions thereof for diagnostic, molecular biological, and other purposes.

### BACKGROUND

DNA polymerases are a class of enzymes (EC 2.7.7.7) that synthesize complementary DNA strands using deoxyribonucleotide substrates. There are multiple types of DNA polymerases displaying different properties in each organism as well as different DNA polymerases in different organisms. DNA polymerases have fundamental roles in DNA replication and repair, functions that are essential for maintaining genetic integrity. In addition to their biological roles, DNA polymerases are essential tools for DNA manipulation, including among others DNA cloning, sequencing, labeling, mutagenesis, detection, and diagnostics. Although all DNA polymerases have the ability to synthesize a deoxyribonucleotide chain, different polymerases have varying properties, including differences in stability (e.g., thermal and/or chemical stability), processivity, fidelity, nucleotide selectivity, sensitivity, and template selectivity (e.g., DNA, RNA, etc.).

While many different DNA polymerase are available, it is desirable to have DNA polymerases that display certain attributes, for example certain levels of stability, processivity, fidelity, nucleotide selectivity, sensitivity, and template selectivity suitable for different uses, such as for use as tools in molecular biology and diagnostics. For example, some DNA polymerases do not effectively use RNA as a template, thereby requiring use of a separate enzyme, a reverse transcriptase, to synthesize a DNA complementary to the RNA template that can then be recognized by the DNA polymerase. The requirement for use of another enzyme with the DNA polymerase inserts additional steps and complexity in diagnostics for detecting a target RNA.

### SUMMARY

The present disclosure provides engineered DNA polymerase polypeptides and compositions thereof, as well as polynucleotides encoding the engineered DNA polymerase polypeptides, where the engineered DNA polymerases have DNA polymerase and reverse transcriptase activity, i.e., capable of using DNA and RNA as templates. The present disclosure also provides methods of using the engineered DNA polymerase polypeptides and compositions thereof for diagnostic and other purposes.

The engineered DNA polymerases of the present disclosure are based on the large fragment (SEQ ID NO: 2) of the full length wild-type DNA polymerase of Parageobacillus genomosp. 1 (SEQ ID NO: 540), where the large fragment includes the DNA polymerase domain but lacks the 5′-exonuclease domain.

In one aspect, the present disclosure provides an engineered DNA polymerase, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of SEQ ID NO: 2, 10, 80, 224, or 366, or to a reference sequence corresponding to SEQ ID NO: 2, 10, 80, 224 or 366, wherein the amino acid sequence comprises one or more substitutions relative to the reference sequence corresponding to amino acid residues 12 to 604 of SEQ ID NO: 2, 10, 80, 224, or 366, or relative to the reference sequence corresponding to SEQ ID NO: 2, 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 2, or to the reference sequence corresponding to SEQ ID NO: 2, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of an even-numbered SEQ ID NO. of SEQ ID NOs: 4-488, or to a reference sequence corresponding to an even-numbered SEQ ID NO. of SEQ ID NOs: 4-488, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the amino acid sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192,210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 2.

In some embodiments, the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 2.

In some embodiments, the amino acid sequence of the engineered DNA polymerase comprises at least a substitution at amino acid position 36, 241, 372, or 470, or combinations thereof, wherein the amino acid positions are relative to the reference sequence corresponding to SEQ ID NO: 2.

In some embodiments, the amino acid sequence of the engineered DNA polymerase comprises at least a substitution or substitution set at amino acid position(s) 509, 300, 452, 36/241/372/470, 124/192/210/372/427/456/552, 124, 52, 483, 372, 393, 212, 52/66, 133, 454, 154, 593, 462, 541, 21, 573, 505, 152, 294, 545, 101/241/470, 584, 304, 295, 578, 456/470, 253, 290, 192/241/372/456, or 252, wherein the amino acid positions are relative to the reference sequence corresponding to SEQ ID NO: 2.

In some embodiments, the amino acid sequence of the engineered DNA polymerase comprises at least one substitution of an engineered DNA polymerase variant set forth in Tables 5.1, 6.1, 7.1, 8.1, and 9.1, wherein the amino acid positions are relative to the reference sequence corresponding to SEQ ID NO: 2.

In some embodiments, the amino acid sequence of the engineered DNA polymerase comprises at least a substitution or substitution set of an engineered DNA polymerase variant set forth in Tables 5.1, 6.1, 7.1, 8.1, and 9.1, wherein the amino acid positions are relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 4-488, or to the reference sequence corresponding to an even-numbered SEQ ID NO. of SEQ ID NOs: 4-488.

In some embodiments, the engineered DNA polymerase comprises an amino acid sequence having at least 70%, 75%, 80%, 81%, 82%, 83%, 84%, 85%, 86%, 87%, 88%, 89% 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 4-488, or to the reference sequence corresponding to an even-numbered SEQ ID NO. of SEQ ID NOs: 4-488, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the amino acid sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192,210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 10, 80, 224, or 366.

In some embodiments, the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 10, 80, 224, or 366.

In some embodiments, the amino acid sequence of the engineered DNA polymerase comprises at least a substitution at amino acid position 36, 241, 372, or 470, or combinations thereof, wherein the amino acid positions are relative to the reference sequence corresponding to SEQ ID NO: 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, or to the reference sequence corresponding to SEQ ID NO: 10, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 10-218, or to the reference sequence corresponding to an even-numbered SEQ ID NO. of SEQ ID NOs: 10-218, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the amino acid sequence of the engineered DNA polymerase comprises at least a substitution set at amino acid positions 52/101/124/212/294/372/393/452/483/509, 52/124/300/393/452, 154/212/294/300/372/393/452/483/509, 52/101/154/294/300/452/509/593, 52/154/212/294/300/393/452, 212/300/393/452/509, 52/101/212/294/300/393/452/483/509/593, 52/124/294/300/452/509, 124/300/393/452/483/509/593, 101/124/212/452/483/509, 52/393/452/509/593, 212/300/452/509/593, 52/452/509/593, 154/212/300/372/452/509/593, 52/124/212/294/393/452/593, 124/452/509, 152/253/287/505/541/573/584, 154/212/300/452, 152/253/287/304/454/545/573/584, 52/124/154/294/300/372/452/509, 294/300/372/452/509/593, 52/212/452, 124/294/393/452, 52/300/509/593, 152/253/287/454/541/584, 52/101/452/509, 52/154/212/300/372/393/509, 21/253/287/454/584, 52/393/452, 52/124/154/294/509, 101/124/212/300/393, 124/294/300/372/452/509/593, 52/124/154/294/483/593, 52/372/393/452/509/593, 154/300/393/452/593, 52/101/294/372/393/452/509, 21/152/253/287/304/454/505, 52/300/393, 101/124/154/294/452/509, 154/212/372/393/509/593, 21/253/287/454/505/541, 287/454/505/584, 52/124/294/300/372/452, 287/505/541/573, 253/505/584, 84/253/287/456/573, 52/372/509/593, 509/593, 154/212/300/372/509, 52/154/294/509/593, 152/295/505/545/573, 253/541/584, 253/295/304/454/462/573/584, 253/454/505/573/584, 154/212/300, 287/454/545/573, 21/152/287/304/454, 152/253/295/304/541/573, 152/253/287/304/454, 152/454/573, 124/294/372/509/593, 152/253/295/454, 154/300/452, 287/304/505, 152/253/454/505, 253/454/505, 21/253/454/505/545, 152/253/287/505, 505/541/584, or 152/253/454/584, wherein the amino acid positions are relative to the reference sequence corresponding to SEQ ID NO: 10.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 80, or to the reference sequence corresponding to SEQ ID NO: 80, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 220-258, or to the reference sequence corresponding to an even-numbered SEQ ID NO. of SEQ ID NOs: 220-258, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the amino acid sequence of the engineered DNA polymerase comprises at least a substitution or substitution set at amino acid position(s) 300/454/456/541/584, 21/300/454/545/584, 253/300/454/456/584, 154/253/300/454/456/505/573, 21/154/300/454/456/573, 152/300/454/456/505/584/593, 154/253/300/456/505/545/573/584, 253/300/454/456/505/573, 154/253/300/454/456/541/573/584, 154/300/454/505, 21/253/300/454/456/545/573/593, 168/300/454/456/545/573, 253/300/454/505/573, 154/300/505/545/584, 154/253/300/456/541/573/584, 21/154/300/454/456/505/545, 154/253/300/454/505, 21/154/253/300/454/573, 300/454/505/541/584, 300/454/505/541/573, 154/253/300/454, 154/300/454/573, 300/454/456/545/573, 253/300/454/456, 154/253/300/454/545/573, 21/152/253/300/454/456/541/573, 154/253/300/454/456/573, 152/300/454, 154/253/300/454/505/541/593, 21/154/300/454/573/584, 154/300/456/573/593, 152/253/300/454/545/573, 21/300/456/495/505/541/584, 154/300/454/541/584, 253/300/505/541/573/584, 253/300/584, 253/300/454/456/545, 21/253/300/573, 300/454/545, 454/456/541/545/573/584, 152/253/454/505/545/573, 21/300/454/456/495, 300/573, 154/300/454/545, 154/253/300/545/584, 21/300/505, 21/152/253/300/454/456/545, 154/253/300/456/541/573, 154/300/454, 253/300/454/541, 253/454/456/541/584, 21/300/573, 300/454/456, 300/454, 154/253/300/454/456, 154/456/505/593, 454/456/505, 253/454/456/584, 154/253/300, 253/456/505/584, 454/456, 154/454, 584, 432/505/573, 456/584, 21/505/545/584, 21/154/545/573, 454/584, or 154/253/505, or 505, wherein the amino acid positions are relative to the reference sequence corresponding to SEQ ID NO: 80.

In some embodiments, the engineered DNA polymerase comprises an amino acid sequence having at least 70%, 75% 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 224, or to the reference sequence corresponding to SEQ ID NO: 224, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 360-400, or to the reference sequence corresponding to an even-numbered SEQ ID NO. of SEQ ID NOs: 360-400, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the amino acid sequence of the engineered DNA polymerase comprises at least a substitution set at amino acid positions 144/154/505/547/573/584, 154/191/325/505/573/584, 144/154/373/374/505/573/584, 134/136/154/505/547/573/584, 81/144/154/505/547/573/584, 154/505/573/584, 144/154/191/230/322/505/573/584, 68/144/154/505/573/584, 144/154/226/230/505/573/584, 144/154/374/486/505/573/584, 81/114/144/154/505/573/584, 144/154/276/505/573/584, 134/144/154/505/573/584, 144/154/505/573/584, 81/144/154/183/505/547/573/584, 68/81/133/134/144/154/505/547/573/584, 144/154/374/505/573/584, 92/133/144/154/505/573/584, 144/154/373/505/573/584, 114/144/154/505/573/584, 154/505/551/573/584/585, or 81/154/505/547/573/584, wherein the amino acid positions are relative to the reference sequence corresponding to SEQ ID NO: 224.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 366, or to the reference sequence corresponding to SEQ ID NO: 366, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 402-488, or to the reference sequence corresponding to an even-numbered SEQ ID NO. of SEQ ID NOs: 402-488, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the amino acid sequence of the engineered DNA polymerase comprises at least a substitution or substitution set at amino acid position(s) 24/58/432, 58/432/575, 58/115/432/575, 24/115/432/575, 24/25/221/432, 25/58/432/575, 24/25/58/432/575, 221/432/575, 432/575, 24/221/432, 24/115/221/432, 24/115/221/432/575, 432, 24/34/432, 24/432/575, 184/221/432/575, 24/432, 24/25/58/115/432/575, 25/221/432, 25/58/115/432, 221/575, 221/432, 24/58/115/221/575, 24/25/221/432/575, 24/25/58/221/432, 34/58/105/432, 24/25/115/432, 24/221/575, 115/432, 58/221, 58/575, 25/432/575, 25/432, 24/221/432/575, 24/115/432, 24/25/105/432, 25/34/432, 25/105/221/432, 24/25/432, 24/58/575, or 24/25/105/432/575, wherein the amino acid positions are relative to the reference sequence corresponding to SEQ ID NO: 366.

In some embodiments, the amino acid sequence of the engineered DNA polymerase comprises at least one substitution of an engineered DNA polymerase variant set forth in Tables 5.1, 6.1, 7.1, 8.1, and 9.1, wherein the amino acid positions are relative to the reference sequence corresponding to SEQ ID NO: 2, 10, 80, 224, or 366.

In some embodiments, the amino acid sequence of the engineered DNA polymerase comprises at least a substitution or substitution set of an engineered DNA polymerase variant set forth in Tables 5.1, 6.1, 7.1, 8.1, and 9.1, wherein the amino acid positions are relative to the reference sequence corresponding to SEQ ID NO: 2, 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered DNA polymerase variant set forth in Tables 5.1, 6.1, 7.1, 8.1, and 9.1.

In some embodiments, the engineered DNA polymerase comprises an amino acid sequence comprising residues 12 to 604 of an even-numbered SEQ ID NO. of SEQ ID NOs: 4-488, or an amino acid sequence comprising an even-numbered SEQ ID NO. of SEQ ID NOs: 4-488, optionally wherein the polypeptide has 1, 2, 3, 4, 5, 6, 7, 8, 9, or up to 10 substitutions in the polypeptide sequence. In some embodiments, the amino acid sequence of the engineered DNA polymerase has 1, 2, 3, 4, up to 5 substitutions in the amino acid sequence. In some embodiments, the substitutions comprise conservative substitutions.

In some embodiments, the engineered DNA polymerase comprises an amino acid sequence comprising residues 12 to 604 of SEQ ID NO: 10, 80, 224, or 366, or an amino acid sequence comprising SEQ ID NO: 10, 80, 224, or 366, optionally wherein the amino acid sequence has 1, 2, 3, 4, 5, 6, 7, 8, 9, or up to 10 substitutions in the amino acid sequence. In some embodiments, the amino acid sequence has 1, 2, 3, 4, up to 5 substitutions in the amino acid sequence. In some embodiments, the substitutions comprise conservative substitutions.

In some embodiments, the engineered DNA polymerase has DNA polymerase activity, including reverse transcriptase activity, and displays one or more improved properties as compared to a reference DNA polymerase or a reference engineered DNA polymerase. In some embodiments, the engineered DNA polymerase has an improved property selected from i) increased activity, ii) increased stability, iii) increased thermostability, iv) increased processivity, v) increased fidelity, vi) increased sensitivity to input target RNA or DNA, vii) increased product yield in an isothermal amplification reaction, viii) increased salt tolerance, and ix) increased resistance to an inhibitor, or any combinations of i), ii), iii), iv), v), vi), vii), viii), and ix) compared to a reference DNA polymerase. In some embodiments, the reference DNA polymerase has an amino acid sequence corresponding to residues 12 to 604 of SEQ ID NO: 2, 10, 80, 224, or 366, or an amino acid sequence corresponding to SEQ ID NO: 2, 10, 80, 224, or 366. In some embodiments, the reference DNA polymerase has an amino acid sequence corresponding to residues 12 to 604 of SEQ ID NO: 2, or an amino acid sequence corresponding to SEQ ID NO: 2.

In some further embodiments, the engineered DNA polymerase is purified. In some embodiments, the engineered DNA polymerase is provided in solution, or is immobilized on a substrate, such as on a solid substrates or membranes or particles.

In another aspect, the present disclosure provides a recombinant polynucleotide comprising a polynucleotide sequence encoding any of the engineered DNA polymerases disclosed herein.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34 to 1812 of SEQ ID NO: 1, 9, 79, 223, or 365, or to a reference polynucleotide sequence corresponding to SEQ ID NO: 1, 9, 79, 224, or 365, wherein the recombinant polynucleotide encodes an engineered DNA polymerase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34 to 1812 of an odd-numbered SEQ ID NO. of SEQ ID NOs: 3-487, or to a reference polynucleotide sequence corresponding to an odd-numbered SEQ ID NO. of SEQ ID NOs: 3-487, wherein the recombinant polynucleotide encodes an engineered DNA polymerase.

In some embodiments, the polynucleotide sequence of the recombinant polynucleotide encoding an engineered DNA polymerase is codon optimized for expression in an organism or cell type thereof, for example a bacterial cell, fungal cell, insect cell, or mammalian cell.

In some embodiments, the recombinant polynucleotide comprises a polynucleotide sequence comprising nucleotide residues 34 to 1812 of SEQ ID NO: 9, 79, 223, or 365, or comprises a polynucleotide sequence comprising SEQ ID NO: 9, 79, 223, or 365.

In some embodiments, the recombinant polynucleotide comprises a polynucleotide sequence comprising nucleotide residues 34 to 1812 of an odd-numbered SEQ ID NO. of SEQ ID NOs: 3-487, or comprises a polynucleotide sequence comprising an odd-numbered SEQ ID NO. of SEQ ID NOs: 3-487.

In a further aspect, the present disclosure provides expression vectors comprising a recombinant polynucleotide provided herein encoding an engineered DNA polymerase. In some embodiments, the recombinant polynucleotide of the expression vector is operably linked to a control sequence. In some embodiments, the control sequence comprises a promoter, particularly a heterologous promoter.

In another aspect, the present disclosure also provides a host cell transformed with an expression vector or recombinant polynucleotide provided herein. In some embodiments, the host cell is a prokaryotic cell or eukaryotic cell. In some embodiments, the host cell is a bacterial cell, fungal cell, insect cell, or mammalian cell. In some embodiments, the host cell is a bacterial cell, such as E. coli.

In a further aspect, the present disclosure provides a method of producing an engineered DNA polymerase polypeptide, the method comprising culturing a host cell described herein under suitable culture conditions such that at least one engineered DNA polymerase is produced. In some embodiments, the method further comprises recovering or isolating the engineered DNA polymerase from the culture media and/or host cells. In some embodiments, the method further comprises purifying the engineered DNA polymerase.

In a further aspect, the present disclosure provides compositions comprising at least one engineered DNA polymerase disclosed herein, particularly for carrying out in vitro polymerase reactions. In some embodiments, the composition comprises one or more a buffer, DNA polymerase substrate, for example, one or more nucleotide substrates (e.g., dNTPs or analogues thereof) and/or oligonucleotide primer substrate. In some embodiments, the composition comprises at least a DNA or RNA template, for example a target DNA or RNA in a sample.

In another aspect, the present disclosure provides use of the engineered DNA polymerase in methods of preparing a complementary DNA copy of a target nucleic acid, whole or in part. In some embodiments, a method of preparing a complementary DNA of a target DNA or RNA, whole or in part, comprises contacting a target DNA or RNA with an engineered DNA polymerase described herein in presence of appropriate substrates under conditions suitable for DNA polymerase mediated production of a DNA complementary to the target DNA or RNA.

In some embodiments, the engineered DNA polymerase is used to detect a target nucleic acid, the method comprising contacting a sample suspected of containing a target nucleic acid with an engineered DNA polymerase of the present disclosure in presence of appropriate substrates under conditions suitable for DNA polymerase mediated production of a DNA complementary to the target nucleic acid, whole or in part, and detecting presence of the complementary DNA. In some embodiments, the target nucleic acid is DNA or RNA. In some embodiments, the sample is a biological or environmental sample. In some embodiments, detecting the complementary DNA is by amplifying the complementary DNA, such as by isothermal amplification or polymerase chain reaction.

In a further aspect, the present disclosure also provides a kit comprising at least one engineered DNA polymerase disclosed herein. In some embodiments, the kit further comprises one or more of a buffer, nucleotide substrate, and/or oligonucleotide primer substrate. In some embodiments, the kit can further comprise a nucleic acid template, such as a DNA or RNA template. In some embodiments, the kit can include a second DNA polymerase, for example a thermostable DNA polymerase.

### DETAILED DESCRIPTION

The present disclosure provides engineered DNA polymerase polypeptides and compositions thereof, as well as recombinant polynucleotides encoding the engineered DNA polymerase polypeptides. The disclosure also provides methods of using the engineered DNA polymerase polypeptides and compositions thereof for diagnostic, molecular biological, and other purposes. In some embodiments, the engineered DNA polymerase polypeptides described herein can use DNA as a template, or RNA as a template (i.e., reverse transcriptase activity), with one or more of improved properties, including, among others, improved polymerization activity, improved replication fidelity, improved processivity, particularly under conditions involving low concentrations of target DNA or RNA input, increased resistance to inhibitors, and increased salt tolerance.

In some embodiments, the engineered DNA polymerases of the present disclosure are particularly useful in diagnostic and research applications using small amounts of DNA or RNA from samples, including cell-free DNA or RNA, circulating tumor DNA or RNA, DNA or RNA isolated from circulating tumor cells, circulating fetal DNA or RNA, DNA or RNA isolated from virally infected cells, fine-needle aspirates, single cells isolated by FACS (fluorescence activated cell sorting), and environmental samples (e.g., water, sewer eluents, air, etc.). However, it is not intended that the sample used with the present disclosure be limited to any particular sample type, as any suitable sample, including those with low target DNA or RNA concentrations, finds use with the engineered DNA polymerases herein.

### ABBREVIATIONS AND DEFINITIONS

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses.

Furthermore, the headings provided herein are not to be construed as limitations of the various aspects or embodiments of the invention and is to be viewed in reference to the application as a whole. Accordingly, the terms defined immediately below are more fully defined by reference to the application as a whole.

As used herein, the singular “a,” “an,” and “the” include the plural references, unless the context clearly indicates otherwise.

As used herein, the term “comprising” and its cognates are used in their inclusive sense (i.e., equivalent to the term “including” and its corresponding cognates).

It is to be further understood that where description of embodiments use the term “comprising” and its cognates, the embodiments can also be described using language “consisting essentially of” or “consisting of.”

Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glutamine (Gln or Q), glycine (Gly or G) histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” “fusion polypeptide,” “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Polymerase” refers to a class of enzymes (e.g., EC 2.7.7.7) that polymerize nucleoside triphosphates to form polynucleotides. In the present context, polymerases use a template nucleic acid strand to synthesize a complementary nucleic acid strand. In some embodiments, the template strand and synthesized nucleic acid strand can independently be either DNA or RNA, depending on the substrate and template specificity of the polymerase. Polymerases known in the art include, but are not limited to, DNA polymerases (e.g., E. coli DNA poll, T. aquaticus DNA polymerase (Taq), Bst Polymerase), DNA-dependent RNA polymerases, and reverse transcriptases. As used herein, the polymerase is a polypeptide or protein containing sufficient amino acids to carry out a desired enzymatic function of the polymerase. In some embodiments, the polymerase does not contain all of the amino acids found in the native enzyme, but only those which are sufficient to allow the polymerase to carry out a desired catalytic activity, including but not limited to 5′-3′ polymerization, 5′-3′ exonuclease, and 3′-5′ exonuclease activities. In some embodiments, the polymerase is limited to the polymerase domain and does not include exonuclease function.

“DNA polymerase activity,” “synthetic activity,” and “polymerase activity” are used interchangeably herein, and refer to the ability of an enzyme to synthesize new DNA strands by the incorporation of deoxynucleoside triphosphates or analogs thereof. In some embodiments, the DNA polymerase may use a DNA and/or RNA as a template.

“Reverse transcriptase activity” refers to the ability of a DNA polymerase enzyme to synthesize new DNA strands by the incorporation of deoxynucleoside triphosphates or analogs thereof using RNA as a template.

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

In some embodiments, “polynucleotide,” “nucleic acid” and “oligonucleotide” includes polynucleotide or nucleic acid or oligonucleotide analogs, which include, among others, nucleosides linked together via other than standard phosphodiester linkages, such as non-standard linkages of phosphoramidates, phosphorothioates, amides, positively-charged linkages, etc.; nucleosides with modified and/or synthetic nucleobases, for example inosine, xanthine, hypoxanthine, etc.; and/or nucleosides with modified sugar residues, such as 2′-O-alkyl (e.g., 2′-O-methyl, 2′-O-ethyl, etc.), 2′-halo (e.g., 2′-F, 2′-Br, etc.) 2,3-dideoxy, 2′-halo-2′-deoxy, β-D-ribo LNA; α-L-ribo-LNA (locked nucleic acids), etc.

“Duplex” and “ds” refer to a double-stranded nucleic acid (e.g., DNA) molecule comprised of two single-stranded polynucleotides that are complementary in their sequence (A pairs to T, C pairs to G), arranged in an antiparallel 5′ to 3′ orientation, and held together by hydrogen bonds between the nucleobases (e.g., adenine [A], guanine [G], cytosine [C], and thymine [T]) or nucleobase analogs.

“Engineered,” “recombinant,” “non-naturally occurring,” and “variant,” when used with reference to a cell, a polynucleotide, or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215: 403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 2, having a serine at the residue corresponding to X470” (or “a reference sequence corresponding to SEQ ID NO: 2, having a serine at the residue corresponding to position 470”) refers to a reference sequence in which the corresponding residue at position X470 in SEQ ID NO: 2 (e.g., isoleucine), has been changed to serine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered DNA polymerase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X470 as compared to SEQ ID NO: 2” (or a “residue difference at position 470 as compared to SEQ ID NO: 2”) refers to a difference of the amino acid residue at the polypeptide position corresponding to position 470 of SEQ ID NO: 2. Thus, if the reference polypeptide of SEQ ID NO: 2 has a isoleucine at position 470, then a “residue difference at position X470 as compared to SEQ ID NO: 2” refers to an amino acid substitution of any residue other than isoleucine at the position of the polypeptide corresponding to position 470 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X21M/X21V, X21M/V, or 21M/V). The present disclosure includes engineered polypeptide sequences comprising one or more amino acid differences that include either/or both conservative and non-conservative amino acid substitutions, as well as insertions and deletions of amino acids in the sequence.

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant DNA polymerase polypeptides listed in any of the Tables in the Examples. In these substitution sets, the individual substitutions are separated by a semicolon (“;”; e.g., S36T;A241Q;K372E;I470S or abbreviated 36T;241Q;372E;470S) or slash (“/”; e.g., S36T/A241Q/K372E/I470S or abbreviated 36T/241Q/372E/470S). In some embodiments, the “substitution” comprises the deletion of an amino acid, and can be denoted by “−” symbol.

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered polymerase enzyme. Deletions can be directed to the internal portions and/or terminal portions of the polypeptide. In various embodiments, the deletion can comprise a continuous segment or can be discontinuous. Deletions are indicated by “−”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered DNA polymerase of the present invention) and that retains substantially all of the activity of the full-length polypeptide.

“Isolated polypeptide” refers to a polypeptide which is separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant DNA polymerase polypeptides may be present within a cell, present in the cellular medium, or prepared in various forms, such as lysates or isolated preparations. As such, in some embodiments, the recombinant DNA polymerase polypeptides provided herein are isolated polypeptides.

“Substantially pure polypeptide” or “purified polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure DNA polymerase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant DNA polymerase polypeptides are substantially pure polypeptide compositions.

“Improved enzyme property” as used herein refers to an engineered DNA polymerase polypeptide that exhibits an improvement in any enzyme property as compared to a reference DNA polymerase polypeptide, such as the DNA polymerase polypeptide sequence of SEQ ID NO: 2, or another engineered DNA polymerase polypeptide. Improved properties include but are not limited to such properties as increased protein expression, increased thermoactivity, increased thermostability, increased stability, increased enzymatic activity, increased substrate specificity and/or affinity, increased specific activity, increased resistance to substrate and/or end-product inhibition, increased chemical stability, improved salt tolerance, improved solvent stability, increased solubility, increased fidelity, increased processivity, increased inhibitor resistance or tolerance, and altered temperature profile.

“Increased enzymatic activity” and “enhanced catalytic activity” refer to an improved property of the engineered DNA polymerase polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of DNA polymerase) as compared to the reference DNA polymerase enzyme (e.g., wild-type DNA polymerase and/or another engineered DNA polymerase). Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. In some embodiments, the engineered DNA polymerase can be from about 1.1, 1.2, 1.3, 1.4, or 1.5 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring DNA polymerase or another engineered DNA polymerase from which the DNA polymerase polypeptides were derived. In some specific embodiments, the engineered DNA polymerase exhibits improved enzymatic activity in the range of 1.5 to 10 fold, 1.5 to 25 fold, 1.5 to 50 fold, 1.5 to 100 fold or greater, than that of the reference DNA polymerase.

“Hybridization stringency” relates to hybridization conditions, such as washing conditions, in the hybridization of nucleic acids (see, e.g., Sambrook et al., Molecular Cloning: A Laboratory Manual, 3rd Ed., Cold Spring Harbor Laboratory Press, 2001). Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5× Denhart's solution, 5×SSPE, 0.2% SDS at 42 °C, followed by washing in 0.2×SSPE, 0.2% SDS, at 42 °C. “High stringency hybridization” refers generally to conditions that are about 10 °C or less from the thermal melting temperature Tₙ as determined under the solution condition for a defined polynucleotide sequence. In some embodiments, a high stringency condition refers to conditions that permit hybridization of only those nucleic acid sequences that form stable hybrids in 0.018M NaCl at 65 °C (i.e., if a hybrid is not stable in 0.018M NaCl at 65 °C, it will not be stable under high stringency conditions, as contemplated herein). High stringency conditions can be provided, for example, by hybridization in conditions equivalent to 50% formamide, 5× Denhart's solution, 5×SSPE, 0.2% SDS at 42 °C, followed by washing in 0.1×SSPE, and 0.1% SDS at 65 °C. Another high stringency condition comprises hybridizing in conditions equivalent to hybridizing in 5×SSC containing 0.1% (w:v) SDS at 65 °C and washing in 0.1×SSC containing 0.1% SDS at 65 °C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the DNA polymerase enzymes are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, propeptide sequences, promoter sequences, signal peptide sequences, initiation sequences, and transcription terminators. In some embodiments, at a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide and/or the encoded polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” or “suitable conditions” refers to those conditions in the enzymatic conversion reaction solution (e.g., ranges of enzyme loading, substrate loading, temperature, pH, buffers, co-solvents, etc.) under which a DNA polymerase polypeptide of the present disclosure is capable of converting a substrate to the desired product compound. Exemplary “suitable reaction conditions” are provided herein (see, the Examples).

“Product” in the context of an enzymatic conversion process refers to the compound or molecule resulting from the action of the DNA polymerase polypeptide on the substrate.

“Culturing” refers to the growing of a population of cells under suitable conditions using any suitable medium (e.g., liquid, gel, or solid). The cells for culturing can be prokaryotic or eukaryotic cells, such as bacterial, fungal, insect, or mammalian cells.

“Vector” is a recombinant construct for introducing a polynucleotide sequence of interest into a cell. In some embodiments, the vector is an expression vector that is operably linked to a suitable control sequence capable of effecting the expression in a suitable host of the polynucleotide or a polypeptide encoded in the polynucleotide sequence. In some embodiments, an “expression vector” has a promoter sequence operably linked to the polynucleotide sequence (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the expression or production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one DNA polymerase variant). In some embodiments, the host cells are prokaryotic or eukaryotic cells that have been transformed or transfected with vectors constructed using recombinant DNA techniques as known in the art.

“Analogue” means a polypeptide having more than 70% sequence identity but less than 100% sequence identity (e.g., more than 75%, 78%, 80%, 81%, 82%, 83%, 84%, 85%, 86%, 87%, 88%, 89%, 90%, 91%, 92%, 93%, 94%, 95%, 96%, 97%, 98%, 99% sequence identity) with a reference polypeptide. In some embodiments, analogues include non-naturally occurring amino acid residues including, but not limited, to homoarginine, ornithine and norvaline, as well as naturally occurring amino acids. In some embodiments, analogues also include one or more D-amino acid residues and non-peptide linkages between two or more amino acid residues.

“Cell-free DNA” or “cfDNA” refers to DNA circulating freely in the bloodstream and is not contained by or associated with cells. In some embodiments, cell-free DNA comprises DNA originally derived and released from normal somatic or germ line cells, cancer cells, fetal cells, microbial cells, or viruses. “Cell-free RNA” or “cfRNA) refers to RNA circulating freely in the bloodstream and is not contained by or associated with cells. In some embodiments, cell-free RNA comprises RNA originally derived and released from normal somatic or germ line cells, cancer cells, fetal cells, microbial cells, or viruses. Cell-free DNA and cell-free RNA include those contained in exosomes.

“Amplification” refers to nucleic acid replication. In some embodiments, the term refers to replication of specific template nucleic acid.

“Isothermal amplification” refers to nucleic acid amplification not limited by constraint of thermal cycling, such as in PCR.

“Polymerase chain reaction” and “PCR” refer to methods of generating multiple copies of a nucleic acid template of interest in presence of nucleic acid primers by repeated cycles of denaturation, annealing, and primer extension with a polymerase, such as described in “PCR: Methods and Protocols” Methods in Molecular Biology,” Springer Protocols (2017) and “Quantitative Real-Time PCR: Methods and Protocols,” Methods in Molecular Biology, Springer Protocols (2014), hereby incorporated by reference. The sequence of denaturation, annealing and extension constitute a “cycle”. The steps of denaturing, primer annealing, and polymerase extension can be repeated (i.e., multiple cycles are used), to amplify a nucleic acid template. In some embodiments, the process of annealing and extension occur in one-step, sometimes referred to as 2-step PCR. Accordingly, as used herein, the term “PCR” includes many variations of the method, including, among others, qPCR, Hot-start PCR, touchdown PCR, asymmetric PCR, multiplex PCR, long or long range PCR, assembly PCR, and inverse PCR.

“Target” when used in reference to a method employing a DNA polymerase refers to the region of nucleic acid for preparation of a complementary DNA. The “target” is sorted out from other nucleic acids present in the methods using a DNA polymerase. In some embodiments, a “segment” is a region of nucleic acid within the target sequence.

“Target DNA” when used in context of a DNA polymerase refers to the DNA, all or a portion thereof, that is the object for preparation of a complementary DNA copy. The target DNA can be the whole of the DNA sequence or a portion thereof, such as a segment of the DNA sequence.

“Target RNA” refers to the RNA, all or a portion thereof, that is the object for preparation of a complementary DNA copy. The target RNA can be the whole of the RNA sequence or a portion thereof, such as a segment of the RNA sequence.

“Sample template” refers to nucleic acid originating from a sample which is analyzed for the presence of target nucleic acid. In contrast, “background template” refers to nucleic acid other than sample template that may or may not be present within a sample. Background template may be inadvertently included in the sample, it may result from carryover, or may be due to the presence of nucleic acid contaminants from which the target nucleic acid is purified. For example, in some embodiments, nucleic acids from organisms other than those to be detected may be present as background in a test sample. However, it is not intended that the present invention be limited to any specific nucleic acid samples or templates.

“Amplifiable nucleic acid” is used in reference to nucleic acids which may be amplified by any amplification method, including but not limited to, PCR and isothermal amplification. In most embodiments, amplifiable nucleic acids comprise sample templates.

“Amplification product” refer to the resultant compounds (i.e., products) obtained after two or more cycles of a nucleic acid amplification method, for example isothermal amplification or PCR, or as indicated by the context.

“Amplification reagents” refer to those reagents (e.g., deoxyribonucleotide triphosphates, buffer, etc.), needed for amplification except for the primers, nucleic acid template, and the amplification enzyme. Typically, amplification reagents, along with other reaction components are placed and contained in a reaction vessel (e.g., test tube, microwell, etc.). It is not intended that the present invention be limited to any specific amplification reagents, as any suitable reagents find use in the present invention.

“Primer” refers to an oligonucleotide (i.e., a sequence of nucleotides), whether occurring naturally or produced synthetically, recombinantly, or by amplification, which is capable of acting as a point of initiation of nucleic acid synthesis, when placed under conditions in which synthesis of a primer extension product that is complementary to a nucleic acid strand is induced (i.e., in the presence of nucleotides and an inducing agent such as DNA polymerase, and at a suitable temperature and pH). In some embodiments, primers are single-stranded, but in some embodiments, primers are double-stranded. In some embodiments, the primers are of sufficient length to prime the synthesis of extension products in the presence of a nucleic acid polymerase. The exact primer length depends upon many factors, as known to those skilled in the art.

“Fidelity,” when used in reference to a polymerase is intended to refer to the accuracy of template-directed incorporation of complementary bases in a synthesized DNA strand relative to the template strand. Typically, fidelity is measured based on the frequency of incorporation of incorrect bases in the newly synthesized nucleic acid strand. The incorporation of incorrect bases can result in point mutations, insertions, or deletions. Fidelity can be calculated according to any method known in the art (see, e.g., Tindall and Kunkel, Biochem., 1988, 27:6008-6013; and Barnes, Gene, 1992, 112:29-35). A polymerase or polymerase variant can exhibit either high fidelity or low fidelity. As used herein, “high fidelity” refers to polymerases with a frequency of accurate base incorporation that exceeds a predetermined value. As used herein, the term “low fidelity” refers to polymerases with a frequency of accurate base incorporation that is lower than a predetermined value. In some embodiments, the predetermined value is a desired frequency of accurate base incorporation or the fidelity of a known polymerase (i.e., a reference polymerase).

“Altered fidelity” refers to the fidelity of a polymerase variant that differs from the fidelity of the parent polymerase from which the polymerase variant was derived or a reference polymerase. In some embodiments, the altered fidelity is higher than the fidelity of the parent or reference polymerase, while in some other embodiments, the altered fidelity is lower than the fidelity of the parent or reference polymerase. Altered fidelity can be determined by assaying the parent and variant polymerases and comparing their activities using any suitable assay known in the art.

“Processivity” refers to the ability of a nucleic acid modifying enzyme, such as a DNA polymerase, to remain bound to the template or substrate and perform multiple modification reactions. Processivity is generally measured by the number of catalytic events that take place per binding event.

“Altered processivity” refers to the processivity of polymerase, or variants thereof that differ from the processivity of the parent polymerase from which the variant was derived or a reference polymerase. In some embodiments, the altered processivity is higher than the processivity of the parent or reference enzyme, while in some other embodiments, the altered processivity is lower than the processivity of the parent or reference enzyme. Altered processivity can be determined by assaying the parent/reference and variant enzymes and comparing their activities using any suitable assay known in the art.

“Subject” encompasses mammals such as humans, non-human primates, livestock, companion animals, and laboratory animals (e.g., rodents and lagamorphs). It is intended that the term encompass females as well as males.

“Patient” means any subject that is being assessed for, treated for, or is experiencing disease. In some embodiments, a “sample” for reaction with a DNA polymerase is obtained from a patient.

“Sample” as used herein refers to a material or substance for reaction with a nucleic acid polymerase, for example, such as for detecting presence of a target nucleic acid or preparing a DNA copy of a target nucleic acid for sequencing or generation of cDNA libraries. In some embodiments, the sample is a “biological sample,” which refers to sample of biological tissue or fluid. Such samples are typically from humans, but include tissues isolated from non-human primates, domesticated mammals (e.g., cats, dogs, cows, sheep, etc.) or rodents, e.g., mice, and rats, and includes sections of tissues such as biopsy and autopsy samples, frozen sections taken for histological purposes, blood, plasma, serum, sputum, stool, tears, mucus, hair, skin, etc. A “biological sample” also refers to a cell or population of cells or a quantity of tissue or fluid from organisms. In some embodiments, the biological sample has been removed from an animal, but the term “biological sample” can also refer to cells or tissue analyzed in vivo, i.e., without removal from the animal, including cell cultures. Typically, a “biological sample” will contain cells from the animal or of organisms, but the term can also refer to non-cellular biological material, such as non-cellular fractions of blood, saliva, lymph, or urine. Numerous types of biological samples can be used with the enzymes, compositions, and method in the present disclosure, including, but not limited to, a tissue biopsy, a blood sample, a buccal scrape, a saliva sample, or a nipple discharge. As used herein, a “tissue biopsy” refers to an amount of tissue removed from an animal, preferably a human, for diagnostic analysis. In a patient with cancer, tissue may be removed from a tumor, allowing the analysis of cells within the tumor. “Tissue biopsy” can refer to any type of biopsy, such as needle biopsy, fine needle biopsy, surgical biopsy, etc. In some embodiments, a sample can be from environmental sources, by way of example and not limitation, water (e.g., ocean, river, refuse/sewer, etc.), soil, air, vents, or surfaces (e.g., floors, machinery, counters, etc.).

### Engineered DNA Polymerase Polypeptides

In one aspect, the present disclosure provides DNA polymerase variants that recognize DNA and RNA as a template and where the DNA polymerase has been engineered to have one or more improved properties, including among others, enhanced activity, enhanced fidelity, enhanced processivity, enhanced stability, enhanced sensitivity under low template DNA or RNA concentrations, increased resistance to inhibitors, and increased salt tolerance as compared to a reference DNA polymerase. As noted above, the engineered DNA polymerases herein are based on the large fragment of the wild-type DNA polymerase of Parageobacillus genomosp. 1, where the large fragment includes a DNA polymerase domain but lacks the 5′ exonuclease domain. The polynucleotide encoding the large fragment of the DNA polymerase of Parageobacillus genomosp. 1 (i.e., residues 285 to 876 of the full length wild-type sequence corresponding to SEQ ID NO: 540) was used as the starting point for generating and identifying DNA polymerase variants with desired properties.

The engineered DNA polymerase variants described herein are useful in performing polymerase reactions, including preparing a complementary DNA of a target DNA or RNA template, whole or in part, in various methods, such as in sequencing (e.g., NGS sequencing), isothermal amplification, DNA library preparation, and diagnostic methods, such as for detecting a target nucleic acid. These engineered DNA polymerase variants can be used in solution, as well as in immobilized embodiments. In some embodiments, the engineered DNA polymerase can be prepared and used as non-fusion polypeptides or as fusion polypeptides.

In some embodiments, an engineered DNA polymerase, or a functional fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of SEQ ID NO: 2, 10, 80, 224, or 366, or to a reference sequence corresponding to SEQ ID NO: 2, 10, 80, 224, or 366, wherein the amino acid sequence comprises one or more substitutions relative to the reference sequence corresponding to amino acid residues 12 to 604 of SEQ ID NO: 2, 10, 80, 224, or 366, or relative to the reference sequence corresponding to SEQ ID NO: 2, 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 2, or to the reference sequence corresponding to SEQ ID NO: 2, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%⁰, 90%, 91%, 92%, 93%, 94%, 95%, 96%, 97%, 98%, 99%, or more sequence identity to the reference sequence corresponding to amino acid residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of an even-numbered SEQ ID NO. of SEQ ID NOs: 4-488, or to a reference sequence corresponding to an even-numbered SEQ ID NO. of SEQ ID NOs: 4-488, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the amino sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192, 210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 2.

In some embodiments, the amino acid sequence of the engineered DNA polymerase comprises at least a substitution 21M/V, 24M, 25L/M, 34D, 36T, 52G, 58K, 66T, 68G, 81R, 84Q, 92G, 101A, 105S, 114S, 115Y, 124L, 133A, 133R, 134R/T, 136P, 144R, 152R, 154W, 168G, 183P, 184T, 191E/R, 192E, 210A, 212V, 221M, 226K, 230G/Q, 241E/Q, 252T, 253R, 276G, 287R, 290G, 294R, 295K, 300A/G/R, 304L, 322C, 325W, 372E, 373A, 374E, 393L, 427A, 432K/L, 452R/S, 454G, 456T, 462R, 470S, 483K, 486R, 495F, 505H, 509R, 541G/R, 541T, 545K, 547A/H/V, 551K, 552L, 573R/V, 575V, 578F, 584V, 585R, or 593S, or combinations thereof, wherein the amino acid positions are relative to the reference sequence corresponding to SEQ ID NO: 2.

In some embodiments, the amino acid sequence of the engineered DNA polymerase comprises at least a substitution E21M/V, L24M, P25L/M, E34D, S36T, E52G, A58K, A66T, E68G, S81R, L84Q, E92G, G101A, I105S, E114S, L115Y, I124L, Q133A/R, S134R/T, E136P, T144R, S152R, E154W, E168G, R183P, A184T, H191E/R, D192E, S210A, L212V, V221M, E226K, E230G/Q, A241E/Q, Q252T, E253R, R276G, D287R, E290G, P294R, Q295K, E300A/G/R, H304L, V322C, R325W, K372E, P373A, D374E, I393L, R427A, Q432K/L, N452R/S, N454G, P456T, E462R, I470S, Q483K, K486R, L495F, T505H, F509R, D541G/R/T, R545K, K547A/H/V, R551K, M552L, Q573R/V, I575V, E578F, I584V, Q585R, or D593S, or combinations thereof, wherein the amino acid positions are relative to the reference sequence corresponding to SEQ ID NO: 2.

In some embodiments, the amino acid sequence of the engineered DNA polymerase comprises at least a substitution at amino acid position 36, 241, 372, or 470, or combinations thereof, wherein the amino acid positions are relative to the reference sequence corresponding to SEQ ID NO: 2. In some embodiments, the amino acid sequence of the engineered DNA polymerase comprises at least a substitution 36T, 241E/Q, 372E, or 470S, or combinations thereof, wherein the amino acid positions are relative to the reference sequence corresponding to SEQ ID NO: 2. In some embodiments, the amino acid sequence of the engineered DNA polymerase comprises at least a substitution at amino acid position 36. In some embodiments, the amino acid sequence of the engineered DNA polymerase comprises at least a substitution at amino acid position 241. In some embodiments, the amino acid sequence of the engineered DNA polymerase comprises at least a substitution at amino acid position 372. In some embodiments, the amino acid sequence of the engineered DNA polymerase comprises at least a substitution at amino acid position 470.

In some embodiments, the amino acid sequence of the engineered DNA polymerase comprises at least a substitution at amino acid position 52, 101, 124, 212, 294, 372, 393, 452, 483, or 509, or combinations thereof, wherein the amino acid positions are relative to the reference sequence corresponding to SEQ ID NO: 2. In some embodiments, the amino acid sequence of the engineered DNA polymerase comprises at least a substitution 52G, 101A, 124L, 212V, 294R, 372K, 393L, 452R, 483K, or 509R, or combinations thereof, wherein the amino acid positions are relative to the reference sequence corresponding to SEQ ID NO: 2. In some embodiments, the amino acid sequence of the engineered DNA polymerase comprises at least a substitution at amino acid position 52. In some embodiments, the amino acid sequence of the engineered DNA polymerase comprises at least a substitution at amino acid position 101. In some embodiments, the amino acid sequence of the engineered DNA polymerase comprises at least a substitution at amino acid position 124. In some embodiments, the amino acid sequence of the engineered DNA polymerase comprises at least a substitution at amino acid position 212. In some embodiments, the amino acid sequence of the engineered DNA polymerase comprises at least a substitution at amino acid position 294. In some embodiments, the amino acid sequence of the engineered DNA polymerase comprises at least a substitution at amino acid position 372. In some embodiments, the amino acid sequence of the engineered DNA polymerase comprises at least a substitution at amino acid position 393. In some embodiments, the amino acid sequence of the engineered DNA polymerase comprises at least a substitution at amino acid position 452. In some embodiments, the amino acid sequence of the engineered DNA polymerase comprises at least a substitution at amino acid position 483. In some embodiments, the amino acid sequence of the engineered DNA polymerase comprises at least a substitution at amino acid position 509.

In some embodiments, the amino acid sequence of the engineered DNA polymerase comprises at least a substitution at amino acid position 253, 300, 454, 505, or 573, or combinations thereof, wherein the amino acid positions are relative to the reference sequence corresponding to SEQ ID NO: 2. In some embodiments, the amino acid sequence of the engineered DNA polymerase comprises at least a substitution 253R, 300R, 454G, 505H, or 573V, or combinations thereof, wherein the amino acid positions are relative to the reference sequence corresponding to SEQ ID NO: 2. In some embodiments, the amino acid sequence of the engineered DNA polymerase comprises at least a substitution at amino acid position 253. In some embodiments, the amino acid sequence of the engineered DNA polymerase comprises at least a substitution at amino acid position 300. In some embodiments, the amino acid sequence of the engineered DNA polymerase comprises at least a substitution at amino acid position 454. In some embodiments, the amino acid sequence of the engineered DNA polymerase comprises at least a substitution at amino acid position 505. In some embodiments, the amino acid sequence of the engineered DNA polymerase comprises at least a substitution at amino acid position 573.

In some embodiments, the amino acid sequence of the engineered DNA polymerase comprises at least a substitution at amino acid position 134, 136, 154, 505, 547, 573, or 584, or combinations thereof, wherein the amino acid positions are relative to the reference sequence corresponding to SEQ ID NO: 2. In some embodiments, the amino acid sequence of the engineered DNA polymerase comprises at least a substitution 134R, 136P, 154W, 505H, 547H, 573V, or 583V, or combinations thereof, wherein the amino acid positions are relative to the reference sequence corresponding to SEQ ID NO: 2. In some embodiments, the amino acid sequence of the engineered DNA polymerase comprises at least a substitution at amino acid position 134. In some embodiments, the amino acid sequence of the engineered DNA polymerase comprises at least a substitution at amino acid position 136. In some embodiments, the amino acid sequence of the engineered DNA polymerase comprises at least a substitution at amino acid position 154. In some embodiments, the amino acid sequence of the engineered DNA polymerase comprises at least a substitution at amino acid position 505. In some embodiments, the amino acid sequence of the engineered DNA polymerase comprises at least a substitution at amino acid position 547. In some embodiments, the amino acid sequence of the engineered DNA polymerase comprises at least a substitution at amino acid position 573. In some embodiments, the amino acid sequence of the engineered DNA polymerase comprises at least a substitution at amino acid position 584.

In some embodiments, the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 2. In some embodiments, the amino acid sequence of the engineered DNA polymerase comprises at least a substitution 36T, 52G, 101A, 124L, 134R/T, 136P, 154W, 212V, 241E/Q, 253R, 294R, 300A/G/R, 372E/K, 393L, 452R/S, 454G, 456T, 470S, 483K, 505H, 509R, 547A/H/V, 573R/V, or 584V, or combinations thereof, wherein the amino acid positions are relative to the reference sequence corresponding to SEQ ID NO: 2.

In some embodiments, the amino acid sequence of the engineered DNA polymerase comprises at least a substitution or substitution set at amino acid position(s) 509, 300, 452, 36/241/372/470, 124/192/210/372/427/456/552, 124, 52, 483, 372, 393, 212, 52/66, 133, 454, 154, 593, 462, 541, 21, 573, 505, 152, 294, 545, 101/241/470, 584, 304, 295, 578, 456/470, 253, 290, 192/241/372/456, or 252, wherein the amino acid positions are relative to the reference sequence corresponding to SEQ ID NO: 2.

In some embodiments, the amino acid sequence of the engineered DNA polymerase comprises at least a substitution or substitution set 509R, 300R, 452R, 36T/241Q/372E/470S, 124L/192E/210A/372E/427A/456T/552L, 124L, 52G, 483K, 372E, 452S, 300A, 393L, 300G, 212V, 52G/66T, 133R, 454G, 154W, 593S, 462R, 541R, 21M, 573V, 505H, 152R, 294R, 545K, 101A/241Q/470S, 584V, 304L, 295K, 578F, 541T, 456T/470S, 253R, 290G, 192E/241E/372E/456T, or 252T, wherein the amino acid positions are relative to the reference sequence corresponding to SEQ ID NO: 2.

In some embodiments, the amino acid sequence of the engineered DNA polymerase comprises at least a substitution or substitution set F509R, E300R, N452R, S36T/A241Q/K372E/I470S, I124L/D192E/S210A/K372E/P427A/P456T/M552L, I124L, E52G, Q483K, K372E, N452S, E300A, I393L, E300G, I212V, E52G/A66T, Q133R, N454G, E154W, D593S, E462R, D541R, E21M, Q573V, T505H, S152R, P294R, R545K, G101A/A241Q/I470S, I584V, H304L, Q295K, E578F, D541T, P456T/I470S, E253R, E290G, D192E/A241E/K372E/P456T, or Q252T, wherein the amino acid positions are relative to the reference sequence corresponding to SEQ ID NO: 2.

In some embodiments, the amino acid sequence of the engineered DNA polymerase comprises at least a substitution set at amino acid positions 36/52/101/124/212/241/294/393/452/470/483/509, 36/241/372/470, 36/154/212/241/294/300/393/452/470/483/509, 36/52/101/154/241/294/300/372/452/470/509/593, 36/52/154/212/241/294/300/372/393/452/470, 36/212/241/300/372/393/452/470/509, 36/52/101/212/241/294/300/372/393/452/470/483/509/593, 36/52/124/241/294/300/372/452/470/509, 36/124/241/300/372/393/452/470/483/509/593, 36/101/124/212/241/372/452/470/483/509, 36/52/393/241/372/452/470/509/593, 36/212/241/300/372/452/470/509/593, 36/52/241/372/452/470/509/593, 36/154/212/241/300/452/470/509/593, 36/52/124/212/241/294/372/393/452/470/593, 36/124/241/372/452/470/509, 36/152/241/253/287/372/470/505/541/573/584, 36/154/212/241/300/372/452/470, 36/152/241/253/287/304/372/454/470/545/573/584, 36/52/124/154/241/294/300/452/470/509, 36/241/294/300/452/470/509/593, 36/52/212/241/372/452/470, 36/124/241/294/372/393/452/470, 36/52/241/300/372/470/509/593, 36/152/241/253/287/372/454/470/541/584, 36/52/101/241/372/452/470/509, 36/52/154/212/241/300/393/470/509, 21/36/241/253/287/372/454/470/584, 36/52/393/241/372/452/470, 36/52/124/154/241/294/372/470/509, 36/101/124/212/241/300/372/393/470, 36/124/241/294/300/452/470/509/593, 36/52/124/154/241/294/372/470/483/593, 36/52/241/393/452/470/509/593, 36/154/241/300/372/393/452/470/593, 36/52/101/241/294/393/452/470/509, 21/36/152/241/253/287/304/372/454/470/505, 36/52/241/300/372/393/470, 36/101/124/154/241/294/372/452/470/509, 36/154/212/241/393/470/509/593, 21/36/241/253/287/372/454/470/505/541, 36/241/287/372/454/470/505/584, 36/52/124/241/294/300/452/470, 36/241/287/372/470/505/541/573, 36/241/253/372/470/505/584, 36/84/241/253/287/372/456/470/573, 36/52/241/470/509/593, 36/241/372/470/509/593, 36/154/212/241/300/470/509, 36/52/154/241/294/372/470/509/593, 36/152/241/295/372/470/505/545/573, 36/241/253/372/470/541/584, 36/241/253/295/304/372/454/462/470/573/584, 36/241/253/372/454/470/505/573/584, 36/154/212/241/300/372/470, 36/241/287/372/454/470/545/573, 21/36/152/241/287/304/372/454/470, 36/152/241/253/295/304/372/470/541/573, 36/152/241/253/287/304/372/470/454, 36/152/241/372/454/470/573, 36/124/241/294/470/509/593, 36/152/241/253/295/372/454/470, 36/154/241/300/372/452/470, 36/241/287/304/372/470/505, 36/152/241/253/372/454/470/505, 36/241/253/372/454/470/505, 21/36/241/253/372/454/470/505/545, 36/152/241/253/287/372/470/505, 36/241/372/470/505/541/584, or 36/152/241/253/372/454/470/584, wherein the amino acid positions are relative to the reference sequence corresponding to SEQ ID NO: 2.

In some embodiments, the amino acid sequence of the engineered DNA polymerase comprises at least a substitution set 36T/52G/101A/124L/212V/241Q/294R/393L/452R/470S/483K/509R, 36T/241Q/372E/470S, 36T/154W/212V/241Q/294R/300G/393L/452R/470S/483K/509R, 36T/52G/101A/154W/241Q/294R/300R/372E/452S/470S/509R/593S, 36T/52G/154W/212V/241Q/294R/300G/372E/393L/452R/470S, 36T/212V/241Q/300R/372E/393L/452R/470S/509R, 36T/52G/101A/212V/241Q/294R/300R/372E/393L/452R/470S/483K/509R/593S, 36T/52G/124L/241Q/294R/300R/372E/452S/470S/509R, 36T/124L/241Q/300R/372E/393L/452R/470S/483K/509R/593S, 36T/101A/124L/212V/241Q/372E/452R/470S/483K/509R, 36T/52G/393L/241Q/372E/452R/470S/509R/593S, 36T/212V/241Q/300R/372E/452R/470S/509R/593S, 36T/52G/241Q/372E/452R/470S/509R/593S, 36T/154W/212V/241Q/300R/452R/470S/509R/593S, 36T/52G/124L/212V/241Q/294R/372E/393L/452R/470S/593S, 36T/124L/241Q/372E/452R/470S/509R, 36T/152R/241Q/253R/287R/372E/470S/505H/541R/573V/584V, 36T/154W/212V/241Q/300R/372E/452R/470S, 36T/152R/241Q/253R/287R/304L/372E/454G/470S/545K/573V/584V, 36T/52G/124L/154W/241Q/294R/300A/452R/470S/509R, 36T/241Q/294R/300R/452S/470S/509R/593, 36T/52G/212V/241Q/372E/452R/470S, 36T/124L/241Q/294R/372E/393L/452R/470S, 36T/52G/241Q/300R/372E/470S/509R/593S, 36T/152R/241Q/253R/287R/372E/454G/470S/541R/584V, 36T/52G/101A/241Q/372E/452S/470S/509R, 36T/52G/154W/212V/241Q/300R/393L/470S/509R, 21M/36T/241Q/253R/287R/372E/454G/470S/584V, 36T/52G/393L/241Q/372E/452R/470S, 36T/52G/124L/154W/241Q/294R/372E/470S/509R, 36T/101A/124L/212V/241Q/300R/372E/393L/470S, 36T/124L/241Q/294R/300G/452S/470S/509R/593S, 36T/52G/124L/154W/241Q/294R/372E/470S/483K/593S, 36T/52G/241Q/393L/452S/470S/509R/593S, 36T/154W/241Q/300R/372E/393L/452S/470S/593S, 36T/52G/101A/241Q/294R/393L/452S/470S/509R, 21M/36T/152R/241Q/253R/287R/304L/372E/454G/470S/505H, 36T/52G/241Q/300R/372E/393L/470S, 36T/101A/124L/154W/241Q/294R/372E/452S/470S/509R, 36T/154W/212V/241Q/393L/470S/509R/593S, 21M/36T/241Q/253R/287R/372E/454G/470S/505H/541G, 36T/241Q/287R/372E/454G/470S/505H/584V, 36T/52G/124L/241Q/294R/300R/452S/470S, 36T/241Q/287R/372E/470S/505H/541R/573V, 36T/241Q/253R/372E/470S/505H/584V, 36T/84Q/241Q/253R/287R/372E/456T/470S/573V, 36T/52G/241Q/470S/509R/593S, 36T/241Q/372E/470S/509R/593S, 36T/154W/212V/241Q/300R/470S/509R, 36T/52G/154W/241Q/294R/372E/470S/509R/593S, 36T/152R/241Q/295K/372E/470S/505H/545K/573V, 36T/241Q/253R/372E/470S/541R/584V, 36T/241Q/253R/295K/304L/372E/454G/462R/470S/573V/584V, 36T/241Q/253R/372E/454G/470S/505H/573V/584V, 36T/154W/212V/241Q/300R/372E/470S, 36T/241Q/287R/372E/454G/470S/545K/573V, 21M/36T/152R/241Q/287R/304L/372E/454G/470S, 36T/152R/241Q/253R/295K/304L/372E/470S/541R/573V, 36T/152R/241Q/253R/287R/304L/372E/470S/454G, 36T/152R/241Q/372E/454G/470S/573V, 36T/124L/241Q/294R/470S/509R/593S, 36T/152R/241Q/253R/295K/372E/454G/470S, 36T/154W/241Q/300R/372E/452S/470S, 36T/241Q/287R/304L/372E/470S/505H, 36T/152R/241Q/253R/372E/454G/470S/505H, 36T/241Q/253R/372E/454G/470S/505H, 21M/36T/241Q/253R/372E/454G/470S/505H/545K, 36T/152R/241Q/253R/287R/372E/470S/505H, 36T/241Q/372E/470S/505H/541R/584V, or 36T/152R/241Q/253R/372E/454G/470S/584V, wherein the amino acid positions are relative to the reference sequence corresponding to SEQ ID NO: 2.

In some embodiments, amino acid sequence of the engineered DNA polymerase comprises at least a substitution or substitution set provided in Table 6.1 relative to the reference sequence corresponding to SEQ ID NO: 2.

In some embodiments, the amino acid sequence of the engineered DNA polymerase comprises at least a substitution set at amino acid positions 36/52/101/124/212/241/294/300/393/452/454/456/470S/483/509/541/584, 21/36/52/101/124/212/241/294/300/393/452/454/470/483/509/545/584, 36/52/101/124/212/241/253/294/300/393/452/454/456/470/483/509/584, 36/52/101/124/154/212/241/253/294/300/393/452/454/456/470/483/505/509/573, 21/36/52/101/124/154/212/241/294/300/393/452/454/456/470/483/509/573, 36/52/101/124/152/212/241/294/300/393/452/454/456/470/483/505/509/584/593, 36/52/101/124/154/212/241/253/294/300/393/452/456/470/483/505/509/545/573/584, 36/52/101/124/212/241/253/294/300/393/452/454/456/470/483/505/509/573, 36/52/101/124/154/212/241/253/294/300/393/452/454/456/470/483/509/541/573/584, 36/52/101/124/154/212/241/294/300/393/452/454/470/483/505/509, 21/36/52/101/124/212/241/253/294/300/393/452/454/456/470/483/509/545/573/593, 36/52/101/124/168/212/241/294/300/393/452/454/456/470/483/509/545/573, 36/52/101/124/212/241/253/294/300/393/452/454/470/483/505/509/573, 36/52/101/124/154/212/241/294/300/393/452/470/483/505/509/545/584, 36/52/101/124/154/212/241/253/294/300/393/452/456/470/483/509/541/573/584, 21/36/52/101/124/154/212/241/294/300/393/452/454/456/470/483/505/509/545, 36/52/101/124/154/212/241/253/294/300/393/452/454/470/483/505/509, 21/36/52/101/124/154/212/241/253/294/300/393/452/454/470/483/509/573, 36/52/101/124/212/241/294/300/393/452/454/470/483/505/509/541/584, 36/52/101/124/212/241/294/300/393/452/454/470/483/505/509/541/573, 36/52/101/124/154/212/241/253/294/300/393/452/454/470/483/509, 36/52/101/124/154/212/241/294/300/393/452/454/470/483/509/573, 36/52/101/124/212/241/294/300/393/452/454/456/470/483/509/545/573, 36/52/101/124/212/241/253/294/300/393/452/454/456/470/483/509, 36/52/101/124/154/212/241/253/294/300/393/452/454/470/483/509/545/573, 21/36/52/101/124/152/212/241/253/294/300/393/452/454/456/470/483/509/541/573, 36/52/101/124/154/212/241/253/294/300/393/452/454/456/470/483/509/573, 36/52/101/124/152/212/241/294/300/393/452/454/470/483/509, 36/52/101/124/154/212/241/253/294/300/393/452/454/470/483/505/509/541/593, 21/36/52/101/124/154/212/241/294/300/393/452/454/470/483/509/573/584, 36/52/101/124/154/212/241/294/300/393/452/456/470/483/509/573/593, 36/52/101/124/152/212/241/253/294/300/393/452/454/470/483/509/545/573, 21/36/52/101/124/212/241/294/300/393/452/456/470/483/495F/505/509/541/584, 36/52/101/124/154/212/241/294/300/393/452/454/470/483/509/541/584, 36/52/101/124/212/241/253/294/300/393/452/470/483/505/509/541/573/584, 36/52/101/124/212/241/253/294/300/393/452/470/483/509/584, 36/52/101/124/212/241/253/294/300/393/452/454/456/470/483/509/545, 21/36/52/101/124/212/241/253/294/300/393/452/470/483/509/573, 36/52/101/124/212/241/294/300/393/452/454/470/483/509/545, 36/52/101/124/212/241/294/393/452/454/456/470/483/509/541/545/573/584, 36/52/101/124/152/212/241/253/294/393/452/454/470/483/505/509/545/573, 21/36/52/101/124/212/241/294/300/393/452/454/456/470/483/495/509, 36/52/101/124/212/241/294/300/393/452/470/483/509/573, 36/52/101/124/154/212/241/294/300/393/452/454/470/483/509/545, 36/52/101/124/154/212/241/253/294/300/393/452/470/483/509/545/584, 21/36/52/101/124/212/241/294/300/393/452/470/483/505/509, 21/36/52/101/124/152/212/241/253/294/300/393/452/454/456/470/483/509/545, 36/52/101/124/154/212/241/253/294/300/393/452/456/470/483/509/541/573, 36/52/101/124/154/212/241/294/300/393/452/454/470/483/509, 36/52/101/124/212/241/253/294/300/393/452/454/470/483/509/541, 36/52/101/124/212/241/253/294/393/452/454/456/470/483/509/541/584, 21/36/52/101/124/212/241/294/300/393/452/470/483/509/573, 36/52/101/124/212/241/294/300/393/452/454/456/470/483/509, 36/52/101/124/212/241/294/300/393/452/454/470/483/509, 36/52/101/124/154/212/241/253/294/300/393/452/454/456/470/483/509, 36/52/101/124/154/212/241/294/393/452/456/470/483/505/509/593, 36/52/101/124/212/241/294/393/452/454/456/470/483/505/509, 36/52/101/124/212/241/253/294/393/452/454/456/470/483/509/584, 36/52/101/124/154/212/241/253/294/300/393/452/470/483/509, 36/52/101/124/212/241/253/294/393/452/456/470/483/505/509/584, 36/52/101/124/212/241/294/393/452/454/456/470/483/509, 36/52/101/124/154/212/241/294/393/452/454/470/483/509, 36/52/101/124/212/241/294/393/452/470/483/509/584, 36/52/101/124/212/241/294/393/452/456/470/483/509/584, 21/36/52/101/124/212/241/294/393/452/470/483/505/509/545/584, 21/36/52/101/124/154/212/241/294/393/452/470/483/509/545/573, 36/52/101/124/212/241/294/393/452/454/470/483/509/584, or 36/52/101/124/154/212/241/253/294/393/452/470/483/505/509, wherein the amino acid positions are relative to the reference sequence corresponding to SEQ ID NO: 2.

In some embodiments, the amino acid sequence of the engineered DNA polymerase comprises at least a substitution set 36T/52G/101A/124L/212V/241Q/294R/300R/393L/452R/454G/456T/470S/483K/509R/541R/584V, 21M/36T/52G/101A/124L/212V/241Q/294R/300R/393L/452R/454G/470S/483K/509R/545K/584V, 36T/52G/101A/124L/212V/241Q/253R/294R/300R/393L/452R/454G/456T/470S/483K/509R/584V, 36T/52G/101A/124L/154W/212V/241Q/253R/294R/300R/393L/452R/454G/456T/470S/483K/505H/509R/573V, 21M/36T/52G/101A/124L/154W/212V/241Q/294R/300R/393L/452R/454G/456T/470S/483K/509R/573V 36T/52G/101A/124L/152R/212V/241Q/294R/300R/393L/452R/454G/456T/470S/483K/505H/509R/584V/593S, 36T/52G/101A/124L/154W/212V/241Q/253R/294R/300R/393L/452R/456T/470S/483K/505H/509R/545K/573V/584V, 36T/52G/101A/124L/212V/241Q/253R/294R/300R/393L/452R/454G/456T/470S/483K/505H/509R/573V 36T/52G/101A/124L/154W/212V/241Q/253R/294R/300R/393L/452R/454G/456T/470S/483K/509R/541 G/573V/584V, 36T/52G/101A/124L/154W/212V/241Q/294R/300R/393L/452R/454G/470S/483K/505H/509R, 21M/36T/52G/101A/124L/212V/241Q/253R/294R/300R/393L/452R/454G/456T/470S/483K/509R/545K/573V/593S, 36T/52G/101A/124L/168G/212V/241Q/294R/300R/393L/452R/454G/456T/470S/483K/509R/545K/573V, 36T/52G/101A/124L/212V/241Q/253R/294R/300R/393L/452R/454G/470S/483K/505H/509R/573V, 36T/52G/101A/124L/154W/212V/241Q/294R/300R/393L/452R/470S/483K/505H/509R/545K/584V, 36T/52G/101A/124L/154W/212V/241Q/253R/294R/300R/393L/452R/456T/470S/483K/509R/541R/573V/584V, 21M/36T/52G/101A/124L/154W/212V/241Q/294R/300R/393L/452R/454G/456T/470S/483K/505H/509R/545K, 36T/52G/101A/124L/154W/212V/241Q/253R/294R/300R/393L/452R/454G/470S/483K/505H/509R, 21M/36T/52G/101A/124L/154W/212V/241Q/253R/294R/300R/393L/452R/454G/470S/483K/509R/573V, 36T/52G/101A/124L/212V/241Q/294R/300R/393L/452R/454G/470S/483K/505H/509R/541R/584V, 36T/52G/101A/124L/212V/241Q/294R/300R/393L/452R/454G/470S/483K/505H/509R/541R/573V, 36T/52G/101A/124L/154W/212V/241Q/253R/294R/300R/393L/452R/454G/470S/483K/509R, 36T/52G/101A/124L/154W/212V/241Q/294R/300R/393L/452R/454G/470S/483K/509R/573V, 36T/52G/101A/124L/212V/241Q/294R/300R/393L/452R/454G/456T/470S/483K/509R/545K/573V, 36T/52G/101A/124L/212V/241Q/253R/294R/300R/393L/452R/454G/456T/470S/483K/509R, 36T/52G/101A/124L/154W/212V/241Q/253R/294R/300R/393L/452R/454G/470S/483K/509R/545K/573V, 21M/36T/52G/101A/124L/S152R/212V/241Q/253R/294R/300R/393L/452R/454G/456T/470S/483K/509R/541R/573V, 36T/52G/101A/124L/154W/212V/241Q/253R/294R/300R/393L/452R/454G/456T/470S/483K/509R/573V, 36T/52G/101A/124L/S152R/212V/241Q/294R/300R/393L/452R/454G/470S/483K/509R, 36T/52G/101A/124L/154W/212V/241Q/253R/294R/300R/393L/452R/454G/470S/483K/505H/509R/541R/593S, 21M/36T/52G/101A/124L/154W/212V/241Q/294R/300R/393L/452R/454G/470S/483K/509R/573V/584V, 36T/52G/101A/124L/154W/212V/241Q/294R/300R/393L/452R/456T/470S/483K/509R/573V/593S, 36T/52G/101A/124L/S152R/212V/241Q/253R/294R/300R/393L/452R/454G/470S/483K/509R/545K/573V, 21M/36T/52G/101A/124L/212V/241Q/294R/300R/393L/452R/456T/470S/483K/L495F/505H/509R/541R/584V, 36T/52G/101A/124L/154W/212V/241Q/294R/300R/393L/452R/454G/470S/483K/509R/541G/584V, 36T/52G/101A/124L/212V/241Q/253R/294R/300R/393L/452R/470S/483K/505H/509R/541R/573V/584V, 36T/52G/101A/124L/212V/241Q/253R/294R/300R/393L/452R/470S/483K/509R/584V, 36T/52G/101A/124L/212V/241Q/253R/294R/300R/393L/452R/454G/456T/470S/483K/509R/545K, 21M/36T/52G/101A/124L/212V/241Q/253R/294R/300R/393L/452R/470S/483K/509R/573V, 36T/52G/101A/124L/212V/241Q/294R/300R/393L/452R/454G/470S/483K/509R/545K, 36T/52G/101A/124L/212V/241Q/294R/393L/452R/454G/456T/470S/483K/509R/541G/545K/573V/584V 36T/52G/101A/124L/S152R/212V/241Q/253R/294R/393L/452R/454G/470S/483K/505H/509R/545K/573V, 21M/36T/52G/101A/124L/212V/241Q/294R/300R/393L/452R/454G/456T/470S/483K/L495F/509R, 36T/52G/101A/124L/212V/241Q/294R/300R/393L/452R/470S/483K/509R/573V, 36T/52G/101A/124L/154W/212V/241Q/294R/300R/393L/452R/454G/470S/483K/509R/545K, 36T/52G/101A/124L/154W/212V/241Q/253R/294R/300R/393L/452R/470S/483K/509R/545K/584V, 21M/36T/52G/101A/124L/212V/241Q/294R/300R/393L/452R/470S/483K/505H/509R, 21M/36T/52G/101A/124L/S152R/212V/241Q/253R/294R/300R/393L/452R/454G/456T/470S/483K/509R/545K, 36T/52G/101A/124L/154W/212V/241Q/253R/294R/300R/393L/452R/456T/470S/483K/509R/541R/573V, 36T/52G/101A/124L/154W/212V/241Q/294R/300R/393L/452R/454G/470S/483K/509R, 36T/52G/101A/124L/212V/241Q/253R/294R/300R/393L/452R/454G/470S/483K/509R/541R, 36T/52G/101A/124L/212V/241Q/253R/294R/393L/452R/454G/456T/470S/483K/509R/541G/584V, 21M/36T/52G/101A/124L/212V/241Q/294R/300R/393L/452R/470S/483K/509R/573V, 36T/52G/101A/124L/212V/241Q/294R/300R/393L/452R/454G/456T/470S/483K/509R, 36T/52G/101A/124L/212V/241Q/294R/300R/393L/452R/454G/470S/483K/509R, 36T/52G/101A/124L/154W/212V/241Q/253R/294R/300R/393L/452R/454G/456T/470S/483K/509R, 36T/52G/101A/124L/154W/212V/241Q/294R/393L/452R/456T/470S/483K/505H/509R/593S, 36T/52G/101A/124L/212V/241Q/294R/393L/452R/454G/456T/470S/483K/505H/509R, 36T/52G/101A/124L/212V/241Q/253R/294R/393L/452R/454G/456T/470S/483K/509R/584V, 36T/52G/101A/124L/154W/212V/241Q/253R/294R/300R/393L/452R/470S/483K/509R, 36T/52G/101A/124L/212V/241Q/253R/294R/393L/452R/456T/470S/483K/505H/509R/584V, 36T/52G/101A/124L/212V/241Q/294R/393L/452R/454G/456T/470S/483K/509R, 36T/52G/101A/124L/154W/212V/241Q/294R/393L/452R/454G/470S/483K/509R, 36T/52G/101A/124L/212V/241Q/294R/393L/452R/470S/483K/509R/584V, 36T/52G/101A/124L/212V/241Q/294R/393L/452R/470S/483K/509R/584V, 36T/52G/101A/124L/212V/241Q/294R/393L/452R/456T/470S/483K/509R/584V, 21M/36T/52G/101A/124L/212V/241Q/294R/393L/452R/470S/483K/505H/509R/545K/584V, 21V/36T/52G/101A/124L/154W/212V/241Q/294R/393L/452R/470S/483K/509R/545K/573V, 36T/52G/101A/124L/212V/241Q/294R/393L/452R/454G/470S/483K/509R/584V, or 36T/52G/101A/124L/154W/212V/241Q/253R/294R/393L/452R/470S/483K/505H/509R, wherein the amino acid positions are relative to the reference sequence corresponding to SEQ ID NO: 2.

In some embodiments, the amino acid sequence of the engineered DNA polymerase comprises at least a substitution or substitution set provided in Table 7.1 relative to the reference sequence corresponding to SEQ ID NO: 2.

In some embodiments, the amino acid sequence of the engineered DNA polymerase comprises at least a substitution set at amino acid positions 36/52/101/124/144/154/212/241/253/294/300/393/452/454/456/470/483/505/509/547/573, 36/52/101/124/154/191/212/241/253/294/300/325/393/452/454/456/470/483/505/509/573, 36/52/101/124/144/154/212/241/253/294/300/373/374/393/452/454/456/470/483/505/509/573, 36/52/101/124/134/136/154/212/241/253/294/300/393/452/454/456/470/483/505/509/547/573, 36/52/81/101/124/144/154/212/241/253/294/300/393/452/454/456/470/483/505/509/547/573, 36/52/101/124/154/212/241/253/294/300/393/452/454/456/470/483/505/509/573, 36/52/101/124/144/154/191/212/230/241/253/294/300/322/393/452/454/456/470/483/505/509/573, 36/52/68/101/124/144/154/212/241/253/294/300/393/452/454/456/470/483/505/509/573, 36/52/101/124/144/154/212/226/230/241/253/294/300/393/452/454/456/470/483/505/509/573, 36/52/101/124/144/154/212/241/253/294/300/374/393/452/454/456/470/483/325/505/509/573, 36/52/81/101/114S/124/144/154/212/241/253/294/300/393/452/454/456/470/483/505/509/573, 36/52/101/124/144/154/212/241/253/276/294/300/393/452/454/456/470/483/505/509/573, 36/52/101/124/134/144/154/212/241/253/294/300/393/452/454/456/470/483/505/509/573, 36/52/101/124/144/154/212/241/253/294/300/393/452/454/456/470/483/505/509/573, 36/52/81/101/124/144/154/183/212/241/253/294/300/393/452/454/456/470/483/505/509/547/573, 36/52/68/81/101/124/133/134/144/154/212/241/253/294/300/393/452/454/456/470/483/505/509/547/573, 36/52/101/124/144/154/212/241/253/294/300/374/393/452/454/456/470/483/505/509/573, 36/52/92/101/124/133/144/154/212/241/253/294/300/393/452/454/456/470/483/505/509/573, 36/52/101/124/144/154/212/241/253/294/300/373/393/452/454/456/470/483/505/509/573, 36/52/101/114/124/144/154/212/241/253/294/300/393/452/454/456/470/483/505/509/573, 36/52/101/124/154/212/241/253/294/300/393/452/454/456/470/483/505/509/551/573/585, or 36/52/81/101/124/154/212/241/253/294/300/393/452/454/456/470/483/505/509/547/573, wherein the amino acid positions are relative to the reference sequence corresponding to SEQ ID NO: 2.

In some embodiments, the amino acid sequence of the engineered DNA polymerase comprises a substitution set 36T/52G/101A/124L/144R/154W/212V/241Q/253R/294R/300R/393L/452R/454G/456T/470S/483K/505H/509R/547H/573V, 36T/52G/101A/124L/154W/191E/212V/241Q/253R/294R/300R/325W/393L/452R/454G/456T/470S/483K/505H/509R/573V, 36T/52G/101A/124L/144R/154W/212V/241Q/253R/294R/300R/373A/374E/393L/452R/454G/456T/470S/483K/505H/509R/573V, 36T/52G/101A/124L/S134R/136P/154W/212V/241Q/253R/294R/300R/393L/452R/454G/456T/470S/483K/505H/509R/547H/573V, 36T/52G/81R/101A/124L/144R/154W/212V/241Q/253R/294R/300R/393L/452R/454G/456T/470S/483K/505H/509R/547H/573V, 36T/52G/101A/124L/154W/212V/241Q/253R/294R/300R/393L/452R/454G/456T/470S/483K/505H/509R/573V, 36T/52G/101A/124L/144R/154W/191R/212V/230G/241Q/253R/294R/300R/322C/393L/452R/454G/456 T/470S/483K/505H/509R/573V, 36T/52G/68G/101A/124L/144R/154W/212V/241Q/253R/294R/300R/393L/452R/454G/456T/470S/483K/505H/509R/573V, 36T/52G/101A/124L/144R/154W/212V/226K/230Q/241Q/253R/294R/300R/393L/452R/454G/456T/470 S/483K/505H/509R/573V, 36T/52G/101A/124L/144R/154W/212V/241Q/253R/294R/300R/374E/393L/452R/454G/456T/470S/483K/486R/505H/509R/573V, 36T/52G/81R/101A/114S/124L/144R/154W/212V/241Q/253R/294R/300R/393L/452R/454G/456T/470S/483K/505H/509R/573V, 36T/52G/101A/124L/144R/154W/212V/241Q/253R/276G/294R/300R/393L/452R/454G/456T/470S/483K/505H/509R/573V, 36T/52G/101A/124L/S134T/144R/154W/212V/241Q/253R/294R/300R/393L/452R/454G/456T/470S/483K/505H/509R/573V, 36T/52G/101A/124L/144R/154W/212V/241Q/253R/294R/300R/393L/452R/454G/456T/470S/483K/505H/509R/573V, 36T/52G/81R/101A/124L/144R/154W/183P/212V/241Q/253R/294R/300R/393L/452R/454G/456T/470S/483K/505H/509R/547A/573V, 36T/52G/68G/81R/101A/124L/133A/S134R/144R/154W/212V/241Q/253R/294R/300R/393L/452R/454G/456T/470S/483K/505H/509R/547H/573V, 36T/52G/101A/124L/144R/154W/212V/241Q/253R/294R/300R/374E/393L/452R/454G/456T/470S/483K/505H/509R/573V, 36T/52G/92G/101A/124L/133A/144R/154W/212V/241Q/253R/294R/300R/393L/452R/454G/456T/470S/483K/505H/509R/573V, 36T/52G/101A/124L/144R/154W/212V/241Q/253R/294R/300R/373A/393L/452R/454G/456T/470S/483K/505H/509R/573V, 36T/52G/101A/114S/124L/144R/154W/212V/241Q/253R/294R/300R/393L/452R/454G/456T/470S/483K/505H/509R/573V, 36T/52G/101A/124L/154W/212V/241Q/253R/294R/300R/393L/452R/454G/456T/470S/483K/505H/509R/551K/573R/585R, or 36T/52G/81R/101A/124L/154W/212V/241Q/253R/294R/300R/393L/452R/454G/456T/470S/483K/505H/509R/547V/573V, wherein the amino acid positions are relative to the reference sequence corresponding to SEQ ID NO: 2.

In some embodiments, amino acid sequence of the engineered DNA polymerase comprises at least a substitution or substitution set provided in Table 8.1 relative to the reference sequence corresponding to SEQ ID NO: 2.

In some embodiments, the amino acid sequence of the engineered DNA polymerase comprises at least a substitution set at amino acid positions 24/36/52/58/101/124/134/136/154/212/241/253/294/300/393/432/452/454/456/470/483/505/509/547/573, 36/52/58/101/124/134/136/154/212/241/253/294/300/393/432/452/454/456/470/483/505/509/547/573/575, 36/52/58/101/115/124/134/136/154/212/241/253/294/300/393/432/452/454/456/470/483/505/509/547/573/575, 24/36/52/101/115/124/134/136/154/212/241/253/294/300/393/432/452/454/456/470/483/505/509/547/573/575, 24/25/36/52/101/124/134/136/154/212/221/241/253/294/300/393/432/452/454/456/470/483/505/509/547/573, 25/36/52/58/101/124/134/136/154/212/241/253/294/300/393/432/452/454/456/470/483/505/509/547/573/575, 24/25/36/52/58/101/124/134/136/154/212/241/253/294/300/393/432/452/454/456/470/483/505/509/547/573/575, 36/52/101/124/134/136/154/212/221/241/253/294/300/393/432/452/454/456/470/483/505/509/547/573/575, 36/52/101/124/134/136/154/212/241/253/294/300/393/432/452/454/456/470/483/505/509/547/573/575, 24/36/52/101/124/134/136/154/212/221/241/253/294/300/393/432/452/454/456/470/483/505/509/547/573, 24/36/52/101/115/124/134/136/154/212/221/241/253/294/300/393/432/452/454/456/470/483/505/509/547/573, 24/36/52/101/115/124/134/136/154/212/221/241/253/294/300/393/432/452/454/456/470/483/505/509/547/573/575, 36/52/101/124/134/136/154/212/241/253/294/300/393/432/452/454/456/470/483/505/509/547/573, 24/34/36/52/101/124/134/136/154/212/241/253/294/300/393/432/452/454/456/470/483/505/509/547/573, 24/36/52/101/124/134/136/154/212/241/253/294/300/393/432/452/454/456/470/483/505/509/547/573/575, 36/52/101/124/134/136/154/184/212/221/241/253/294/300/393/432/452/454/456/470/483/505/509/547/573/575, 24/36/52/101/124/134/136/154/212/241/253/294/300/393/432/452/454/456/470/483/505/509/547/573, 24/25/36/52/58/101/115/124/134/136/154/212/241/253/294/300/393/432/452/454/456/470/483/505/509/547/573/575, 25/36/52/101/124/134/136/154/212/221/241/253/294/300/393/432/452/454/456/470/483/505/509/547/573, 25/36/52/58/101/115/124/134/136/154/212/241/253/294/300/393/432/452/454/456/470/483/505/509/547/573, 36/52/101/124/134/136/154/212/221/241/253/294/300/393/452/454/456/470/483/505/509/547/573/575, 36/52/101/124/134/136/154/212/221/241/253/294/300/393/432/452/454/456/470/483/505/509/547/573, 24/36/52/58/101/115/124/134/136/154/212/221/241/253/294/300/393/452/454/456/470/483/505/509/547/573/575, 24/25/36/52/101/124/134/136/154/212/221/241/253/294/300/393/432/452/454/456/470/483/505/509/547/573/575, 24/25/36/52/58/101/124/134/136/154/212/221/241/253/294/300/393/432/452/454/456/470/483/505/509/547/573, 34/36/52/58/101/105/124/134/136/154/212/241/253/294/300/393/432/452/454/456/470/483/505/509/547/573, 24/25/36/52/101/115/124/134/136/154/212/241/253/294/300/393/432/452/454/456/470/483/505/509/547/573, 24/36/52/101/124/134/136/154/212/221/241/253/294/300/393/452/454/456/470/483/505/509/547/573/575, 36/52/101/115/124/134/136/154/212/241/253/294/300/393/432/452/454/456/470/483/505/509/547/573, 36/52/58/101/124/134/136/154/212/221/241/253/294/300/393/452/454/456/470/483/505/509/547/573, 36/52/58/101/124/134/136/154/212/241/253/294/300/393/452/454/456/470/483/505/509/547/573/575, 25/36/52/101/124/134/136/154/212/241/253/294/300/393/432/452/454/456/470/483/505/509/547/573/575, 25/36/52/101/124/134/136/154/212/241/253/294/300/393/432/452/454/456/470/483/505/509/547/573, 24/36/52/101/124/134/136/154/212/221/241/253/294/300/393/432/452/454/456/470/483/505/509/547/573/575, 24/36/52/101/115/124/134/136/154/212/241/253/294/300/393/432/452/454/456/470/483/505/509/547/573, 24/25/36/52/101/105/124/134/136/154/212/241/253/294/300/393/432/452/454/456/470/483/505/509/547/573, 25/34/36/52/101/124/134/136/154/212/241/253/294/300/393/432/452/454/456/470/483/505/509/547/573, 25/36/52/101/105/124/134/136/154/212/221/241/253/294/300/393/432/452/454/456/470/483/505/509/547/573, 24/25/36/52/101/124/134/136/154/212/241/253/294/300/393/432/452/454/456/470/483/505/509/547/573, 24/36/52/58/101/124/134/136/154/212/241/253/294/300/393/452/454/456/470/483/505/509/547/573/575, or 24/25/36/52/101/105/124/134/136/154/212/241/253/294/300/393/432/452/454/456/470/483/505/509/547/573/575, wherein the amino acid positions are relative to the reference sequence corresponding to SEQ ID NO: 2.

In some embodiments, the amino acid sequence of the engineered DNA polymerase comprises at least a substitution set 24M/36T/52G/58K/101A/124L/134R/136P/154W/212V/241Q/253R/294R/300R/393L/432L/452R/454G/456T/470S/483K/505H/509R/547H/573V, 36T/52G/58K/101A/124L/134R/136P/154W/212V/241Q/253R/294R/300R/393L/432L/452R/454G/456T/470S/483K/505H/509R/547H/573V/575V, 36T/52G/58K/101A/115Y/124L/134R/136P/154W/212V/241Q/253R/294R/300R/393L/432L/452R/454G/456T/470S/483K/505H/509R/547H/573V/575V, 24M/36T/52G/101A/115Y/124L/134R/136P/154W/212V/241Q/253R/294R/300R/393L/432L/452R/454G/456T/470S/483K/505H/509R/547H/573V/575V, 24M/25L/36T/52G/101A/124L/134R/136P/154W/212V/221M/241Q/253R/294R/300R/393L/432L/452R/454G/456T/470S/483K/505H/509R/547H/573V, 25L/36T/52G/58K/101A/124L/134R/136P/154W/212V/241Q/253R/294R/300R/393L/432L/452R/454G/456T/470S/483K/505H/509R/547H/573V/575V, 24M/25M/36T/52G/58K/101A/124L/134R/136P/154W/212V/241Q/253R/294R/300R/393L/432L/452R/454G/456T/470S/483K/505H/509R/547H/573V/575V, 36T/52G/101A/124L/134R/136P/154W/212V/221M/241Q/253R/294R/300R/393L/432L/452R/454G/456 T/470S/483K/505H/509R/547H/573V/575V, 36T/52G/101A/124L/134R/136P/154W/212V/241Q/253R/294R/300R/393L/432L/452R/454G/456T/470S/483K/505H/509R/547H/573V/575V, 24M/36T/52G/101A/124L/134R/136P/154W/212V/221M/241Q/253R/294R/300R/393L/432L/452R/454G/456T/470S/483K/505H/509R/547H/573V, 24M/36T/52G/101A/115Y/124L/134R/136P/154W/212V/221M/241Q/253R/294R/300R/393L/432L/452R/454G/456T/470S/483K/505H/509R/547H/573V, 24M/25M/36T/52G/101A/124L/134R/136P/154W/212V/221M/241Q/253R/294R/300R/393L/432L/452R/454G/456T/470S/483K/505H/509R/547H/573V, 24M/36T/52G/101A/115Y/124L/134R/136P/154W/212V/221M/241Q/253R/294R/300R/393L/432L/452R/454G/456T/470S/483K/505H/509R/547H/573V/575V, 36T/52G/101A/124L/134R/136P/154W/212V/241Q/253R/294R/300R/393L/432L/452R/454G/456T/470S/483K/505H/509R/547H/573V, 24M/34D/36T/52G/101A/124L/134R/136P/154W/212V/241Q/253R/294R/300R/393L/432L/452R/454G/456T/470S/483K/505H/509R/547H/573V, 24M/36T/52G/101A/124L/134R/136P/154W/212V/241Q/253R/294R/300R/393L/432L/452R/454G/456T/470S/483K/505H/509R/547H/573V/575V, 36T/52G/101A/124L/134R/136P/154W/184T/212V/221M/241Q/253R/294R/300R/393L/432L/452R/454 G/456T/470S/483K/505H/509R/547H/573V/575V, 24M/36T/52G/101A/124L/134R/136P/154W/212V/241Q/253R/294R/300R/393L/432L/452R/454G/456T/470S/483K/505H/509R/547H/573V, 24M/25L/36T/52G/58K/101A/115Y/124L/134R/136P/154W/212V/241Q/253R/294R/300R/393L/432L/452R/454G/456T/470S/483K/505H/509R/547H/573V/575V, 25L/36T/52G/101A/124L/134R/136P/154W/212V/221M/241Q/253R/294R/300R/393L/432L/452R/454G/456T/470S/483K/505H/509R/547H/573V, 25M/36T/52G/58K/101A/115Y/124L/134R/136P/154W/212V/241Q/253R/294R/300R/393L/432L/452R/454G/456T/470S/483K/505H/509R/547H/573V, 36T/52G/101A/124L/134R/136P/154W/212V/221M/241Q/253R/294R/300R/393L/452R/454G/456T/470 S/483K/505H/509R/547H/573V/575V, 36T/52G/101A/124L/134R/136P/154W/212V/221M/241Q/253R/294R/300R/393L/432L/452R/454G/456 T/470S/483K/505H/509R/547H/573V, 24M/36T/52G/58K/101A/115Y/124L/134R/136P/154W/212V/221M/241Q/253R/294R/300R/393L/452R/454G/456T/470S/483K/505H/509R/547H/573V/575V, 24M/25M/36T/52G/101A/124L/134R/136P/154W/212V/221M/241Q/253R/294R/300R/393L/432L/452R/454G/456T/470S/483K/505H/509R/547H/573V/575V, 24M/25M/36T/52G/58K/101A/124L/134R/136P/154W/212V/221M/241Q/253R/294R/300R/393L/432L/452R/454G/456T/470S/483K/505H/509R/547H/573V, 34D/36T/52G/58K/101A/105S/124L/134R/136P/154W/212V/241Q/253R/294R/300R/393L/432L/452R/454G/456T/470S/483K/505H/509R/547H/573V, 24M/25M/36T/52G/101A/115Y/124L/134R/136P/154W/212V/241Q/253R/294R/300R/393L/432L/452R/454G/456T/470S/483K/505H/509R/547H/573V, 24M/36T/52G/101A/124L/134R/136P/154W/212V/221M/241Q/253R/294R/300R/393L/452R/454G/456T/470S/483K/505H/509R/547H/573V/575V, 36T/52G/101A/115Y/124L/134R/136P/154W/212V/241Q/253R/294R/300R/393L/432L/452R/454G/456T/470S/483K/505H/509R/547H/573V, 36T/52G/58K/101A/124L/134R/136P/154W/212V/221M/241Q/253R/294R/300R/393L/452R/454G/456T/470S/483K/505H/509R/547H/573V, 36T/52G/58K/101A/124L/134R/136P/154W/212V/241Q/253R/294R/300R/393L/452R/454G/456T/470S/483K/505H/509R/547H/573V/575V, 25L/36T/52G/101A/124L/134R/136P/154W/212V/241Q/253R/294R/300R/393L/432L/452R/454G/456T/470S/483K/505H/509R/547H/573V/575V, 25L/36T/52G/101A/124L/134R/136P/154W/212V/241Q/253R/294R/300R/393L/432L/452R/454G/456T/470S/483K/505H/509R/547H/573V, 24M/36T/52G/101A/124L/134R/136P/154W/212V/221M/241Q/253R/294R/300R/393L/432L/452R/454G/456T/470S/483K/505H/509R/547H/573V/575V, 24M/36T/52G/101A/115Y/124L/134R/136P/154W/212V/241Q/253R/294R/300R/393L/432L/452R/454G/456T/470S/483K/505H/509R/547H/573V, 24M/25M/36T/52G/101A/105S/124L/134R/136P/154W/212V/241Q/253R/294R/300R/393L/432L/452R/454G/456T/470S/483K/505H/509R/547H/573V, 25M/34D/36T/52G/101A/124L/134R/136P/154W/212V/241Q/253R/294R/300R/393L/432L/452R/454G/456T/470S/483K/505H/509R/547H/573V, 24M/25L/36T/52G/101A/105S/124L/134R/136P/154W/212V/241Q/253R/294R/300R/393L/432L/452R/454G/456T/470S/483K/505H/509R/547H/573V, 25M/36T/52G/101A/124L/134R/136P/154W/212V/241Q/253R/294R/300R/393L/432L/452R/454G/456T/470S/483K/505H/509R/547H/573V, 25M/36T/52G/101A/105S/124L/134R/136P/154W/212V/221M/241Q/253R/294R/300R/393L/432L/452R/454G/456T/470S/483K/505H/509R/547H/573V, 24M/25L/36T/52G/101A/124L/134R/136P/154W/212V/241Q/253R/294R/300R/393L/432L/452R/454G/456T/470S/483K/505H/509R/547H/573V, 24M/36T/52G/58K/101A/124L/134R/136P/154W/212V/241Q/253R/294R/300R/393L/452R/454G/456T/470S/483K/505H/509R/547H/573V/575V, or 24M/25M/36T/52G/101A/105S/124L/134R/136P/154W/212V/241Q/253R/294R/300R/393L/432L/452R/454G/456T/470S/483K/505H/509R/547H/573V/575V, wherein the amino acid positions are relative to the reference sequence corresponding to SEQ ID NO: 2.

In some embodiments, the amino acid sequence of the engineered DNA polymerase comprises at least a substitution or substitution set provided in Table 9.1 relative to the reference sequence corresponding to SEQ ID NO: 2.

In some embodiments, the engineered DNA polymerase comprises an amino acid sequence having at least 70%, 75%, 80%, 81%, 82%, 83%, 84%, 85%, 86%, 87%, 88%, 89%⁰, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366. In some embodiments, the engineered DNA polymerase includes the proviso that the DNA polymerase having the amino acid sequence corresponding to residues 12 to 604 of SEQ ID NO: 2 is excluded.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 604 of an even-numbered SEQ ID NO. of SEQ ID NOs: 4-488, or to a reference sequence corresponding to an even-numbered SEQ ID NO. of SEQ ID NOs: 4-488.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 4-488, or to the reference sequence corresponding to an even-numbered SEQ ID NO. of SEQ ID NOs: 4-488, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the amino acid sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192,210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 10, 80, 224, or 366.

In some embodiments, the amino acid sequence of the engineered DNA polymerase comprises at least a substitution or an amino acid residue 21M/V, 24M, 25L/M, 34D, 36T, 52G, 58K, 66T, 68G, 81R, 84Q, 92G, 101A, 105S, 114S, 115Y, 124L, 133A, 133R, 134R/T, 136P, 144R, 152R, 154W, 168G, 183P, 184T, 191E/R, 192E, 210A, 212V, 221M, 226K, 230G/Q, 241E/Q, 252T, 253R, 276G, 287R, 290G, 294R, 295K, 300A/G/R, 304L, 322C, 325W, 372E/K, 373A, 374E, 393L, 427A, 432K/L, 452R/S, 454G, 456T, 462R, 470S, 483K, 486R, 495F, 505H, 509R, 541G/R/T, 545K, 547A/H/V, 551K, 552L, 573R/V, 575V, 578F, 584V/I, 585R, or 593S, or combinations thereof, wherein the amino acid positions are relative to the reference sequence corresponding to SEQ ID NO: 10, 80, 224, or 366.

In some embodiments, the amino acid sequence of the engineered DNA polymerase comprises at least a substitution at amino acid position 36, 241, 372, or 470, or combinations thereof, wherein the amino acid positions are relative to the reference sequence corresponding to SEQ ID NO: 10, 80, 224, or 366. In some embodiments, the amino acid sequence of the engineered DNA polymerase comprises a substitution or an amino acid residue 36T, 241Q, 372K/E, or 470S, or combinations thereof, wherein the amino acid positions are relative to the reference sequence corresponding to SEQ ID NO: 10, 80, 224, or 366.

In some embodiments, the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 10, 80, 224, or 366. In some embodiments, the amino acid sequence of the engineered DNA polymerase comprises a substitution or an amino acid residue 36T, 52G, 101A, 124L, 134R, 136P, 154W, 212V, 241Q, 253R, 294R, 300R, 372K/E, 393L, 452R, 454G, 456T, 470S, 483K, 505H, 509R, 547H, 573V, or 584V/I, or combinations thereof, wherein the amino acid positions are relative to the reference sequence corresponding to SEQ ID NO: 10, 80, 224, or 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, or to the reference sequence corresponding to SEQ ID NO: 10, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 10-218, or to the reference sequence corresponding to an even-numbered SEQ ID NO. of SEQ ID NOs: 10-218, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the amino acid sequence of the engineered DNA polymerase comprises at least a substitution set at amino acid position(s) 52/101/124/212/294/372/393/452/483/509, 52/124/300/393/452, 154/212/294/300/372/393/452/483/509, 52/101/154/294/300/452/509/593, 52/154/212/294/300/393/452, 212/300/393/452/509, 52/101/212/294/300/393/452/483/509/593, 52/124/294/300/452/509, 124/300/393/452/483/509/593, 101/124/212/452/483/509, 52/393/452/509/593, 212/300/452/509/593, 52/452/509/593, 154/212/300/372/452/509/593, 52/124/212/294/393/452/593, 124/452/509, 152/253/287/505/541/573/584, 154/212/300/452, 152/253/287/304/454/545/573/584, 52/124/154/294/300/372/452/509, 294/300/372/452/509/593, 52/212/452, 124/294/393/452, 52/300/509/593, 152/253/287/454/541/584, 52/101/452/509, 52/154/212/300/372/393/509, 21/253/287/454/584, 52/393/452, 52/124/154/294/509, 101/124/212/300/393, 124/294/300/372/452/509/593, 52/124/154/294/483/593, 52/372/393/452/509/593, 154/300/393/452/593, 52/101/294/372/393/452/509, 21/152/253/287/304/454/505, 52/300/393, 101/124/154/294/452/509, 154/212/372/393/509/593, 21/253/287/454/505/541, 287/454/505/584, 52/124/294/300/372/452, 287/505/541/573, 253/505/584, 84/253/287/456/573, 52/372/509/593, 509/593, 154/212/300/372/509, 52/154/294/509/593, 152/295/505/545/573, 253/541/584, 253/295/304/454/462/573/584, 253/454/505/573/584, 154/212/300, 287/454/545/573, 21/152/287/304/454, 152/253/295/304/541/573, 152/253/287/304/454, 152/454/573, 124/294/372/509/593, 152/253/295/454, 154/300/452, 287/304/505, 152/253/454/505, 253/454/505, 21/253/454/505/545, 152/253/287/505, 505/541/584, or 152/253/454/584, wherein the amino acid positions are relative to the reference sequence corresponding to SEQ ID NO: 10.

In some embodiments, the amino acid sequence of the engineered DNA polymerase comprises at least a substitution set 52G/101A/124L/212V/294R/372K/393L/452R/483K/509R, 52G/124L/300A/393L/452R, 154W/212V/294R/300G/372K/393L/452R/483K/509R, 52G/101A/154W/294R/300R/452S/509R/593S, 52G/154W/212V/294R/300G/393L/452R, 212V/300R/393L/452R/509R, 52G/101A/212V/294R/300R/393L/452R/483K/509R/593S, 52G/124L/294R/300R/452S/509R, 124L/300R/393L/452R/483K/509R/593S, 101A/124L/212V/452R/483K/509R, 52G/393L/452R/509R/593S, 212V/300R/452R/509R/593S, 52G/452R/509R/593S, 154W/212V/300R/372K/452R/509R/593S, 52G/124L/212V/294R/393L/452R/593S, 124L/452R/509R, 152R/253R/287R/505H/541R/573V/584V, 154W/212V/300R/452R, 152R/253R/287R/304L/454G/545K/573V/584V, 52G/124L/154W/294R/300A/372K/452R/509R, 294R/300R/372K/452S/509R/593S, 52G/212V/452R, 124L/294R/393L/452R, 52G/300R/509R/593S, 152R/253R/287R/454G/541R/584V, 52G/101A/452S/509R, 52G/154W/212V/300R/372K/393L/509R, 21M/253R/287R/454G/584V, 52G/393L/452R, 52G/124L/154W/294R/509R, 101A/124L/212V/300R/393L, 124L/294R/300G/372K/452S/509R/593S, 52G/124L/154W/294R/483K/593S, 52G/372K/393L/452S/509R/593S, 154W/300R/393L/452S/593S, 52G/101A/294R/372K/393L/452S/509R, 21M/152R/253R/287R/304L/454G/505H, 52G/300R/393L, 101A/124L/154W/294R/452S/509R, 154W/212V/372K/393L/509R/593S, 21M/253R/287R/454G/505H/541G, 287R/454G/505H/584V, 52G/124L/294R/300R/372K/452S, 287R/505H/541R/573V, 253R/505H/584V, 84Q/253R/287R/456T/573V, 52G/372K/509R/593S, 509R/593S, 154W/212V/300R/372K/509R, 52G/154W/294R/509R/593S, 152R/295K/505H/545K/573V, 253R/541R/584V, 253R/295K/304L/454G/462R/573V/584V, 253R/454G/505H/573V/584V, 154W/212V/300R, 287R/454G/545K/573V, 21M/152R/287R/304L/454G, 152R/253R/295K/304L/541R/573V, 152R/253R/287R/304L/454G, 152R/454G/573V, 124L/294R/372K/509R/593S, 152R/253R/295K/454G, 154W/300R/452S, 287R/304L/505H, 152R/253R/454G/505H, 253R/454G/505H, 21M/253R/454G/505H/545K, 152R/253R/287R/505H, 505H/541R/584V, or 152R/253R/454G/584V, wherein the amino acid positions are relative to the reference sequence corresponding to SEQ ID NO: 10.

In some embodiments, the amino acid sequence of the engineered DNA polymerase comprises at least a substitution set E52G/G101A/I124L/I212V/P294R/E372K/I393L/N452R/Q483K/F509R, E52G/I124L/E300A/I393L/N452R, E154W/I212V/P294R/E300G/E372K/I393L/N452R/Q483K/F509R, E52G/G101A/E154W/P294R/E300R/N452S/F509R/D593S, E52G/E154W/I212V/P294R/E300G/I393L/N452R, I212V/E300R/I393L/N452R/F509R, E52G/G101A/I212V/P294R/E300R/I393L/N452R/Q483K/F509R/D593S, E52G/I124L/P294R/E300R/N452S/F509R, I124L/E300R/I393L/N452R/Q483K/F509R/D593S, G101A/I124L/I212V/N452R/Q483K/F509R, E52G/I393L/N452R/F509R/D593S, I212V/E300R/N452R/F509R/D593S, E52G/N452R/F509R/D593S, E154W/I212V/E300R/E372K/N452R/F509R/D593S, E52G/I124L/I212V/P294R/I393L/N452R/D593S, I124L/N452R/F509R, S152R/E253R/D287R/T505H/D541R/Q573V/I584V, E154W/I212V/E300R/N452R, S152R/E253R/D287R/H304L/N454G/R545K/Q573V/I584V, E52G/I124L/E154W/P294R/E300A/E372K/N452R/F509R, P294R/E300R/E372K/N452S/F509R/D593S, E52G/I212V/N452R, I124L/P294R/I393L/N452R, E52G/E300R/F509R/D593S, S152R/E253R/D287R/N454G/D541R/I584V, E52G/G101A/N452S/F509R, E52G/E154W/I212V/E300R/E372K/I393L/F509R, E21M/E253R/D287R/N454G/I584V, E52G/I393L/N452R, E52G/I124L/E154W/P294R/F509R, G101A/I124L/I212V/E300R/I393L, I124L/P294R/E300G/E372K/N452S/F509R/D593S, E52G/I124L/E154W/P294R/Q483K/D593S, E52G/E372K/I393L/N452S/F509R/D593S, E154W/E300R/I393L/N452S/D593S, E52G/G101A/P294R/E372K/I393L/N452S/F509R, E21M/S152R/E253R/D287R/H304L/N454G/T505H, E52G/E300R/I393L, G101A/I124L/E154W/P294R/N452S/F509R, E154W/I212V/E372K/I393L/F509R/D593S, E21M/E253R/D287R/N454G/T505H/D541G, D287R/N454G/T505H/I584V, E52G/I124L/P294R/E300R/E372K/N452S, D287R/T505H/D541R/Q573V, E253R/T505H/I584V, L84Q/E253R/D287R/P456T/Q573V, E52G/E372K/F509R/D593S, F509R/D593S, E154W/I212V/E300R/E372K/F509R, E52G/E154W/P294R/F509R/D593S, S152R/Q295K/T505H/R545K/Q573V, E253R/D541R/I584V, E253R/Q295K/H304L/N454G/E462R/Q573V/I584V, E253R/N454G/T505H/Q573V/I584V, E154W/I212V/E300R, D287R/N454G/R545K/Q573V, E21M/S152R/D287R/H304L/N454G, S152R/E253R/Q295K/H304L/D541R/Q573V, S152R/E253R/D287R/H304L/N454G, S152R/N454G/Q573V, I124L/P294R/E372K/F509R/D593S, S152R/E253R/Q295K/N454G, E154W/E300R/N452S, D287R/H304L/T505H, S152R/E253R/N454G/T505H, E253R/N454G/T505H, E21M/E253R/N454G/T505H/R545K, S152R/E253R/D287R/T505H, T505H/D541R/I584V, or S152R/E253R/N454G/I584, wherein the amino acid positions are relative to the reference sequence corresponding to SEQ ID NO: 10.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 80, or to the reference sequence corresponding to SEQ ID NO: 80, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 220-258, or to the reference sequence corresponding to an even-numbered SEQ ID NO. of SEQ ID NOs: 220-258, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the amino acid sequence of the engineered DNA polymerase comprises at least a substitution or substitution set at amino acid position(s) 300/454/456/541/584, 21/300/454/545/584, 253/300/454/456/584, 154/253/300/454/456/505/573, 21/154/300/454/456/573, 152/300/454/456/505/584/593, 154/253/300/456/505/545/573/584, 253/300/454/456/505/573, 154/253/300/454/456/541/573/584, 154/300/454/505, 21/253/300/454/456/545/573/593, 168/300/454/456/545/573, 253/300/454/505/573, 154/300/505/545/584, 154/253/300/456/541/573/584, 21/154/300/454/456/505/545, 154/253/300/454/505, 21/154/253/300/454/573, 300/454/505/541/584, 300/454/505/541/573, 154/253/300/454, 154/300/454/573, 300/454/456/545/573, 253/300/454/456, 154/253/300/454/545/573, 21/152/253/300/454/456/541/573, 154/253/300/454/456/573, 152/300/454, 154/253/300/454/505/541/593, 21/154/300/454/573/584, 154/300/456/573/593, 152/253/300/454/545/573, 21/300/456/495/505/541/584, 154/300/454/541/584, 253/300/505/541/573/584, 253/300/584, 253/300/454/456/545, 21/253/300/573, 300/454/545, 454/456/541/545/573/584, 152/253/454/505/545/573, 21/300/454/456/495, 300/573, 154/300/454/545, 154/253/300/545/584, 21/300/505, 21/152/253/300/454/456/545, 154/253/300/456/541/573, 154/300/454, 253/300/454/541, 253/454/456/541/584, 21/300/573, 300/454/456, 300/454, 154/253/300/454/456, 154/456/505/593, 454/456/505, 253/454/456/584, 154/253/300, 253/456/505/584, 454/456, 154/454, 584, 432/505/573, 456/584, 21/505/545/584, 21/154/545/573, 454/584, 154/253/505, or 505, wherein the amino acid positions are relative to the reference sequence corresponding to SEQ ID NO: 80.

In some embodiments, the amino acid sequence of the engineered DNA polymerase comprises at least a substitution or substitution set 300R/454G/456T/541R/584V, 21M/300R/454G/545K/584V, 253R/300R/454G/456T/584V, 154W/253R/300R/454G/456T/505H/573V, 21M/154W/300R/454G/456T/573V, 152R/300R/454G/456T/505H/584V/593S, 154W/253R/300R/456T/505H/545K/573V/584V, 253R/300R/454G/456T/505H/573V, 154W/253R/300R/454G/456T/541G/573V/584V, 154W/300R/454G/505H, 21M/253R/300R/454G/456T/545K/573V/593S, 168G/300R/454G/456T/545K/573V, 253R/300R/454G/505H/573V, 154W/300R/505H/545K/584V, 154W/253R/300R/456T/541R/573V/584V, 21M/154W/300R/454G/456T/505H/545K, 154W/253R/300R/454G/505H, 21M/154W/253R/300R/454G/573V, 300R/454G/505H/541R/584V, 300R/454G/505H/541R/573V, 154W/253R/300R/454G, 154W/300R/454G/573V, 300R/454G/456T/545K/573V, 253R/300R/454G/456T, 154W/253R/300R/454G/545K/573V, 21M/152R/253R/300R/454G/456T/541R/573V, 154W/253R/300R/454G/456T/573V, 152R/300R/454G, 154W/253R/300R/454G/505H/541R/593S, 21M/154W/300R/454G/573V/584V, 154W/300R/456T/573V/593S, 152R/253R/300R/454G/545K/573V, 21M/300R/456T/495F/505H/541R/584V, 154W/300R/454G/541G/584V, 253R/300R/505H/541R/573V/584V, 253R/300R/584V, 253R/300R/454G/456T/545K, 21M/253R/300R/573V, 300R/454G/545K, 454G/456T/541G/545K/573V/584V, 152R/253R/454G/505H/545K/573V, 21M/300R/454G/456T/495F, 300R/573V, 154W/300R/454G/545K, 154W/253R/300R/545K/584V, 21M/300R/505H, 21M/152R/253R/300R/454G/456T/545K, 154W/253R/300R/456T/541R/573V, 154W/300R/454G, 253R/300R/454G/541R, 253R/454G/456T/541G/584V, 21M/300R/573V, 300R/454G/456T, 300R/454G, 154W/253R/300R/454G/456T, 154W/456T/505H/593S, 454G/456T/505H, 253R/454G/456T/584V, 154W/253R/300R, 253R/456T/505H/584V, 454G/456T, 154W/454G, 584V, 432K/505H/573V, 456T/584V, 21M/505H/545K/584V, 21V/154W/545K/573V, 454G/584V, 154W/253R/505H, or 505H, wherein the amino acid positions are relative to the reference sequence corresponding to SEQ ID NO: 80.

In some embodiments, the amino acid sequence of the engineered DNA polymerase comprises at least a substitution or substitution set E300R/N454G/P456T/D541R/I584V, E21M/E300R/N454G/R545K/I584V, E253R/E300R/N454G/P456T/I584V, E154W/E253R/E300R/N454G/P456T/T505H/Q573V, E21M/E154W/E300R/N454G/P456T/Q573V, S152R/E300R/N454G/P456T/T505H/I584V/D593S, E154W/E253R/E300R/P456T/T505H/R545K/Q573V/I584V, E253R/E300R/N454G/P456T/T505H/Q573V, E154W/E253R/E300R/N454G/P456T/D541G/Q573V/I584V, E154W/E300R/N454G/T505H, E21M/E253R/E300R/N454G/P456T/R545K/Q573V/D593S, E168G/E300R/N454G/P456T/R545K/Q573V, E253R/E300R/N454G/T505H/Q573V, E154W/E300R/T505H/R545K/I584V, E154W/E253R/E300R/P456T/D541R/Q573V/I584V, E21M/E154W/E300R/N454G/P456T/T505H/R545K, E154W/E253R/E300R/N454G/T505H, E21M/E154W/E253R/E300R/N454G/Q573V, E300R/N454G/T505H/D541R/I584V, E300R/N454G/T505H/D541R/Q573V, E154W/E253R/E300R/N454G, E154W/E300R/N454G/Q573V, E300R/N454G/P456T/R545K/Q573V, E253R/E300R/N454G/P456T, E154W/E253R/E300R/N454G/R545K/Q573V, E21M/S152R/E253R/E300R/N454G/P456T/D541R/Q573V, E154W/E253R/E300R/N454G/P456T/Q573V, S152R/E300R/N454G, E154W/E253R/E300R/N454G/T505H/D541R/D593S, E21M/E154W/E300R/N454G/Q573V/I584V, E154W/E300R/P456T/Q573V/D593S, S152R/E253R/E300R/N454G/R545K/Q573V, E21M/E300R/P456T/L495F/T505H/D541R/I584V, E154W/E300R/N454G/D541G/I584V, E253R/E300R/T505H/D541R/Q573V/I584V, E253R/E300R/I584V, E253R/E300R/N454G/P456T/R545K, E21M/E253R/E300R/Q573V, E300R/N454G/R545K, N454G/P456T/D541G/R545K/Q573V/I584V, S152R/E253R/N454G/T505H/R545K/Q573V, E21M/E300R/N454G/P456T/L495F, E300R/Q573V, E154W/E300R/N454G/R545K, E154W/E253R/E300R/R545K/I584V, E21M/E300R/T505H, E21M/S152R/E253R/E300R/N454G/P456T/R545K, E154W/E253R/E300R/P456T/D541R/Q573V, E154W/E300R/N454G, E253R/E300R/N454G/D541R, E253R/N454G/P456T/D541G/I584V, E21M/E300R/Q573V, E300R/N454G/P456T, E300R/N454G, E154W/E253R/E300R/N454G/P456T, E154W/P456T/T505H/D593S, N454G/P456T/T505H, E253R/N454G/P456T/I584V, E154W/E253R/E300R, E253R/P456T/T505H/I584V, N454G/P456T, E154W/N454G, I584V, Q432K/T505H/Q573V, P456T/I584V, E21M/T505H/R545K/I584V, E21V/E154W/R545K/Q573V, N454G/I584V, E154W/E253R/T505H, or T505H, wherein the amino acid positions are relative to the reference sequence corresponding to SEQ ID NO: 80.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 224, or to the reference sequence corresponding to SEQ ID NO: 224, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 360-400, or to the reference sequence corresponding to an even-numbered SEQ ID NO. of SEQ ID NOs: 360-400, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the amino acid sequence of the engineered DNA polymerase comprises at least a substitution set at amino acid position(s) 144/154/505/547/573/584, 154/191/325/505/573/584, 144/154/373/374/505/573/584, 134/136/154/505/547/573/584, 81/144/154/505/547/573/584, 154/505/573/584, 144/154/191/230/322/505/573/584, 68/144/154/505/573/584, 144/154/226/230/505/573/584, 144/154/374/486/505/573/584, 81/114/144/154/505/573/584, 144/154/276/505/573/584, 134/144/154/505/573/584, 144/154/505/573/584, 81/144/154/183/505/547/573/584, 68/81/133/134/144/154/505/547/573/584, 144/154/374/505/573/584, 92/133/144/154/505/573/584, 144/154/373/505/573/584, 114/144/154/505/573/584, 154/505/551/573/584/585, or 81/154/505/547/573/584, wherein the amino acid positions are relative to the reference sequence corresponding to SEQ ID NO: 224.

In some embodiments, the amino acid sequence of the engineered DNA polymerase comprises at least a substitution set 144R/154W/505H/547H/573V/584I, 154W/191E/325W/505H/573V/584I, 144R/154W/373A/374E/505H/573V/584I, 134R/136P/154W/505H/547H/573V/584I, 81R/144R/154W/505H/547H/573V/584I, 154W/505H/573V/584I, 144R/154W/191R/230G/322C/505H/573V/584I, 68G/144R/154W/505H/573V/584I, 144R/154W/226K/230Q/505H/573V/584I, 144R/154W/374E/486R/505H/573V/584I, 81R/114S/144R/154W/505H/573V/584I, 144R/154W/276G/505H/573V/584I, 134T/144R/154W/505H/573V/584I, 144R/154W/505H/573V/584I, 81R/144R/154W/183P/505H/547A/573V/584I, 68G/81R/133A/134R/144R/154W/505H/547H/573V/584I, 144R/154W/374E/505H/573V/584I, 92G/133A/144R/154W/505H/573V/584I, 144R/154W/373A/505H/573V/584I, 114S/144R/154W/505H/573V/584I, 154W/505H/551K/573R/584I/585R, or 81R/154W/505H/547V/573V/584I, wherein the amino acid positions are relative to the reference sequence corresponding to SEQ ID NO: 224.

In some embodiments, the amino acid sequence of the engineered DNA polymerase comprises at least substitution set T144R/E154W/T505H/K547H/Q573V/V584I, E154W/H191E/R325W/T505H/Q573V/V584I, T144R/E154W/P373A/D374E/T505H/Q573V/V584I, S134R/E136P/E154W/T505H/K547H/Q573V/V584I, S81R/T144R/E154W/T505H/K547H/Q573V/V584I, E154W/T505H/Q573V/V584I, T144R/E154W/H191R/E230G/V322C/T505H/Q573V/V584I, E68G/T144R/E154W/T505H/Q573V/V584I, T144R/E154W/E226K/E230Q/T505H/Q573V/V584I, T144R/E154W/D374E/K486R/T505H/Q573V/V584I, S81R/E114S/T144R/E154W/T505H/Q573V/V584I, T144R/E154W/K276G/T505H/Q573V/V584I, S134T/T144R/E154W/T505H/Q573V/V584I, T144R/E154W/T505H/Q573V/V584I, S81R/T144R/E154W/R183P/T505H/K547A/Q573V/V584I, E68G/S81R/Q133A/S134R/T144R/E154W/T505H/K547H/Q573V/V584I, T144R/E154W/D374E/T505H/Q573V/V584I, E92G/Q133A/T144R/E154W/T505H/Q573V/V584I, T144R/E154W/P373A/T505H/Q573V/V584I, E114S/T144R/E154W/T505H/Q573V/V584I, E154W/T505H/R551K/Q573R/V584I/Q585R, or S81R/E154W/T505H/K547V/Q573V/V584I, wherein the amino acid positions are relative to the reference sequence corresponding to SEQ ID NO: 224.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 366, or to the reference sequence corresponding to SEQ ID NO: 366, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 402-488, or to the reference sequence corresponding to an even-numbered SEQ ID NO. of SEQ ID NOs: 402-488, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the amino acid sequence of the engineered DNA polymerase comprises at least a substitution or substitution set at amino acid position(s) 24/58/432, 58/432/575, 58/115/432/575, 24/115/432/575, 24/25/221/432, 25/58/432/575, 24/25/58/432/575, 221/432/575, 432/575, 24/221/432, 24/115/221/432, 24/115/221/432/575, 432, 24/34/432, 24/432/575, 184/221/432/575, 24/432, 24/25/58/115/432/575, 25/221/432, 25/58/115/432, 221/575, 221/432, 24/58/115/221/575, 24/25/221/432/575, 24/25/58/221/432, 34/58/105/432, 24/25/115/432, 24/221/575, 115/432, 58/221, 58/575, 25/432/575, 25/432, 24/221/432/575, 24/115/432, 24/25/105/432, 25/34/432, 25/105/221/432, 24/25/432, 24/58/575, or 24/25/105/432/575, wherein the amino acid positions are relative to the reference sequence corresponding to SEQ ID NO: 366.

In some embodiments, the amino acid sequence of the engineered DNA polymerase comprises at least a substitution or substitution set 24M/58K/432L, 58K/432L/575V, 58K/115Y/432L/575V, 24M/115Y/432L/575V, 24M/25L/221M/432L, 25L/58K/432L/575V, 24M/25M/58K/432L/575V, 221M/432L/575V, 432L/575V, 24M/221M/432L, 24M/115Y/221M/432L, 24M/25M/221M/432L, 24M/115Y/221M/432L/575V, 432L, 24M/34D/432L, 24M/432L/575V, 184T/221M/432L/575V, 24M/432L, 24M/25L/58K/115Y/432L/575V, 25L/221M/432L, 25M/58K/115Y/432L, 221M/575V, 221M/432L, 24M/58K/115Y/221M/575V, 24M/25M/221M/432L/575V, 24M/25M/58K/221M/432L, 34D/58K/105S/432L, 24M/25M/115Y/432L, 24M/221M/575V, 115Y/432L, 58K/221M, 58K/575V, 25L/432L/575V, 25L/432L, 24M/221M/432L/575V, 24M/115Y/432L, 24M/25M/105S/432L, 25M/34D/432L, 24M/25L/105S/432L, 25M/432L, 25M/105S/221M/432L, 24M/25L/432L, 24M/58K/575V, or 24M/25M/105S/432L/575V, wherein the amino acid positions are relative to the reference sequence corresponding to SEQ ID NO: 366.

In some embodiments, the amino acid sequence of the engineered DNA polymerase comprises at least a substitution or substitution set L24M/A58K/Q432L, A58K/Q432L/I575V, A58K/L115Y/Q432L/I575V, L24M/L115Y/Q432L/I575V, L24M/P25L/V221M/Q432L, P25L/A58K/Q432L/I575V, L24M/P25M/A58K/Q432L/I575V, V221M/Q432L/I575V, Q432L/I575V, L24M/V221M/Q432L, L24M/L115Y/V221M/Q432L, L24M/P25M/V221M/Q432L, L24M/L115Y/V221M/Q432L/I575V, Q432L, L24M/E34D/Q432L, L24M/Q432L/I575V, A184T/V221M/Q432L/I575V, L24M/Q432L, L24M/P25L/A58K/L115Y/Q432L/I575V, P25L/V221M/Q432L, P25M/A58K/L115Y/Q432L, V221M/I575V, V221M/Q432L, L24M/A58K/L115Y/V221M/I575V, L24M/P25M/V221M/Q432L/I575V, L24M/P25M/A58K/V221M/Q432L, E34D/A58K/I105S/Q432L, L24M/P25M/L115Y/Q432L, L24M/V221M/I575V, L115Y/Q432L, A58K/V221M, A58K/I575V, P25L/Q432L/I575V, P25L/Q432L, L24M/V221M/Q432L/I575V, L24M/L115Y/Q432L, L24M/P25M/I105S/Q432L, P25M/E34D/Q432L, L24M/P25L/I105S/Q432L, P25M/Q432L, P25M/I105S/V221M/Q432L, L24M/P25L/Q432L, L24M/A58K/I575V, or L24M/P25M/I105S/Q432L/I575V, wherein the amino acid positions are relative to the reference sequence corresponding to SEQ ID NO: 366.

In some embodiments, amino acid sequence of the engineered DNA polymerase comprises at least a substitution at an amino acid position provided in Tables 5.1, 6.1, 7.1, 8.1, and 9.1, wherein the substitution is relative to the reference sequence corresponding to SEQ ID NO: 2, 10, 80, 244, or 366, as provided in each of the Tables.

In some embodiments, the amino acid sequence of the engineered DNA polymerase comprises at least one substitution provided in Tables 5.1, 6.1, 7.1, 8.1, and 9.1, wherein the substitution is relative to the reference sequence corresponding to SEQ ID NO: 2, 10, 80, 244, or 366, as provided in each of the Tables.

In some embodiments, the amino acid sequence of the engineered DNA polymerase comprises at least a substitution or substitution set at the amino acid position(s) provided in Tables 5.1, 6.1, 7.1, 8.1, and 9.1, wherein the substitution or substitution set is relative to the reference sequence corresponding to SEQ ID NO: 2, 10, 80, 244, or 366, as provided in each of the Tables.

In some embodiments, the amino acid sequence of the engineered DNA polymerase comprises at least a substitution or substitution set of a DNA polymerase variant provided in Tables 5.1, 6.1, 7.1, 8.1, and 9.1, wherein the substitution or substitution set is relative to the reference sequence corresponding to SEQ ID NO: 2, 10, 80, 244, or 366 as provided in each of the Tables.

In some embodiments, the engineered DNA polymerase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered DNA polymerase variant set forth in Tables 5.1, 6.1, 7.1, 8.1, and 9.1.

In some embodiments, the amino acid sequence of the engineered DNA polymerase comprises residues 12 to 604 of an even-numbered SEQ ID NO. of SEQ ID NOs: 4-488, or comprises an even-numbered SEQ ID NO. of SEQ ID NOs: 4-488. In some embodiments, the amino acid sequence of the engineered DNA polymerase optionally includes 1, 2, 3, 4, 5, 6, 7, 8, 9, or up to 10 substitutions, insertions, and/or deletions. In some embodiments, the amino acid sequence of the engineered DNA polymerase includes 1, 2, 3, 4, 5, 6, 7, 8, 9, or up to 10 substitutions. In some embodiments, the amino acid sequence of the engineered DNA polymerase optionally includes 1, 2, 3, 4, or 5 substitutions, insertions, and/or deletions. In some embodiments, the amino acid sequence of the engineered DNA polymerase optionally includes 1, 2, 3, 4, or 5 substitutions.

In some embodiments, the amino acid sequence of the engineered DNA polymerase comprises residues 12 to 604 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 440, 442, 444, 446, 448, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, or 488. In some embodiments, the amino acid sequence of the engineered DNA polymerase optionally includes 1, 2, 3, 4, 5, 6, 7, 8, 9, or up to 10 substitutions, insertions, and/or deletions. In some embodiments, the amino acid sequence of the engineered DNA polymerase includes 1, 2, 3, 4, 5, 6, 7, 8, 9, or up to 10 substitutions. In some embodiments, the amino acid sequence of the engineered DNA polymerase optionally includes 1, 2, 3, 4, or 5 substitutions, insertions, and/or deletions. In some embodiments, the amino acid sequence of the engineered DNA polymerase optionally includes 1, 2, 3, 4, or 5 substitutions.

In some embodiments, the amino acid sequence of the engineered DNA polymerase comprises SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 440, 442, 444, 446, 448, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, or 488. In some embodiments, the amino acid sequence of the engineered DNA polymerase optionally includes 1, 2, 3, 4, 5, 6, 7, 8, 9, or up to 10 substitutions, insertions, and/or deletions. In some embodiments, the amino acid sequence of the engineered DNA polymerase includes 1, 2, 3, 4, 5, 6, 7, 8, 9, or up to 10 substitutions. In some embodiments, the amino acid sequence of the engineered DNA polymerase optionally includes 1, 2, 3, 4, or 5 substitutions, insertions, and/or deletions. In some embodiments, the amino acid sequence of the engineered DNA polymerase optionally includes 1, 2, 3, 4, or 5 substitutions.

In some embodiments, the engineered DNA polymerase polypeptide has 1, 2, 3, 4, or up to 5 substitutions in the amino acid sequence. In some embodiments, the engineered DNA polymerase polypeptide has 1, 2, 3, or 4 substitutions in the amino acid sequence.

In some embodiments, the substitution comprises conservative substitutions. In some embodiments, the substitution comprises non-conservative substitutions. In some embodiments, the substitutions comprise conservative and non-conservative substitutions. In some embodiments, guidance on non-conservative and conservative substitutions are provided by the variants disclosed herein.

In some embodiments, the engineered DNA polymerase comprises an amino acid sequence comprising residues 12 to 604 of SEQ ID NO: 10, 80, 224, or 366, or the amino acid sequence comprising SEQ ID NO: 10, 80, 224, or 366. In some embodiments, the amino acid sequence of the engineered DNA polymerase optionally includes 1, 2, 3, 4, 5, 6, 7, 8, 9, or up to 10 substitutions, insertions, and/or deletions. In some embodiments, the amino acid sequence of the engineered DNA polymerase includes 1, 2, 3, 4, 5, 6, 7, 8, 9, or up to 10 substitutions. In some embodiments, the amino acid sequence of the engineered DNA polymerase optionally includes 1, 2, 3, 4, or 5 substitutions, insertions, and/or deletions. In some embodiments, the amino acid sequence of the engineered DNA polymerase optionally includes 1, 2, 3, 4, or 5 substitutions.

It will be apparent that the description herein, including the Examples, provide Tables showing sequence structural information correlating specific amino acid sequence features with the functional activity of the engineered DNA polymerase polypeptides. This structure-function correlation information is provided in the form of specific amino acid residue differences relative to the reference engineered DNA polymerase polypeptide of SEQ ID NO: 2, 10, 80, 224, or 366, as well as associated experimentally determined activity data for the exemplary engineered DNA polymerase polypeptides. Such information provide guidance and information on substitutions implemented in preparing engineered DNA polymerase variants.

In some embodiments, the engineered DNA polymerase of the present disclosure has DNA polymerase activity. In some embodiments, the engineered DNA polymerase of the present disclosure has reverse transcriptase activity. In some embodiments, the engineered DNA polymerase of the present disclosure has DNA polymerase activity using DNA and RNA as a template.

In some embodiments, the engineered DNA polymerase has DNA polymerase and/or reverse transcriptase activity and at least one or more improved properties as compared to a reference DNA polymerase. In some embodiments, the engineered DNA polymerase has increased activity as compared to the reference DNA polymerase. In some embodiments, the engineered DNA polymerase has increased stability as compared to the reference DNA polymerase. In some embodiments, the engineered DNA polymerase has increased thermostability as compared to the reference DNA polymerase. In some embodiments, the engineered DNA polymerase has increased processivity as compared to the reference DNA polymerase. In some embodiments, the engineered DNA polymerase has increased fidelity as compared to the reference DNA polymerase. In some embodiments, the engineered DNA polymerase has increased input DNA or RNA (e.g., target) sensitivity as compared to the reference DNA polymerase. In some embodiments, the engineered DNA polymerase has increased product yield in an isothermal amplification reaction as compared to the reference DNA polymerase. In some embodiments, the engineered DNA polymerase has increased salt tolerance as compared to a reference DNA polymerase. In some embodiments, the reference DNA polymerase has the sequence corresponding to residues 12 to 604 of SEQ ID NO: 2, 10, 80, 224, or 366, or the sequence corresponding to SEQ ID NO: 2, 10, 80, 224, or 366. In some embodiments, the reference DNA polymerase has the sequence corresponding to residues 12 to 604 of SEQ ID NO: 2 or the sequence corresponding to SEQ ID NO: 2.

In some embodiments, the engineered DNA polymerase has increased resistance to inhibitors. In some embodiments, the engineered DNA polymerase has increased resistance to inhibitors as compared to the reference DNA polymerase. In some embodiments, the engineered DNA polymerase has increased resistance to ethanol, guanidine thiocyanate, viral transport medium (VTM), heparin, hematin, RNA, or genomic DNA. Exemplary concentrations of inhibitors for assessing increased resistance includes 3% (v/v) ethanol, 25 mM guanidine thiocyanate, 20% (v/v) VTM (see, Preparation of Viral Transport Medium, Centers for Disease Control and Prevention, SOP #: DSR-052-05), 6.25 units/mL heparin, 10 uM hematin, 50 ng total RNA, or 100 ng human genomic DNA. In some embodiments, the reference DNA polymerase has the sequence corresponding to residues 12 to 604 of SEQ ID NO: 2 or the sequence corresponding to SEQ ID NO: 2. In some embodiments, the reference DNA polymerase is Bst 3.0 (New England Biolabs, Catalog #M0374). In some embodiments, the reference DNA polymerase is commercially available as LavaLAMP™ DNA Master Mix (LGC Biosearch Technologies).

In some embodiments, the engineered DNA polymerase has increased input DNA or RNA (e.g., target) sensitivity as compared to the reference DNA polymerase, where the input is about 1250 copies, about 1000 copies, about 750 copies, about 500 copies, about 200, about 100 copies, or about 75 copies of a target RNA or DNA in a volume of 5 uL, for example, as provided in the Examples. In some embodiments, the engineered DNA polymerase has increased product yield in an isothermal amplification reaction as compared to the reference DNA polymerase, where the input target is about 1250 copies, about 1000 copies, about 750 copies, about 500 copies, about 200 or about 100 copies of a target RNA or DNA in a volume of 5 uL, as provided in the Examples.

In some embodiments, the improved property of the engineered DNA polymerase is selected from i) increased activity, ii) increased stability, iii) increased thermostability, iv) increased processivity, v) increased fidelity, vi) increased sensitivity to input target RNA or DNA, vii) increased product yield in an isothermal amplification reaction, viii) increased salt tolerance, and ix) increased resistance to an inhibitor, or any combinations of i), ii), iii), iv), v), vi), vii), viii), and ix) compared to a reference DNA polymerase. In some embodiments, the reference DNA polymerase has the sequence corresponding to residues 12 to 604 of SEQ ID NO: 2, 10, 80, 224, or 366, or the sequence corresponding to SEQ ID NO: 2, 10, 80, 224, or 366. In some embodiments, the reference DNA polymerase has the sequence corresponding to residues 12 to 604 of SEQ ID NO: 2, or the sequence corresponding to SEQ ID NO: 2.

In some embodiments, the DNA polymerase described herein does not have significant or measurable 3′-exonuclease activity. In some embodiments, the DNA polymerase described herein does not have significant or measurable 5′-exonuclease activity. In some embodiments, the DNA polymerase described herein does not have significant or measurable 3′-exonuclease and 5′-exonuclease activities.

Although the engineered DNA polymerases described herein relate to the large fragment lacking a 5′-exonuclease domain, it is to be understood that in some embodiments, the engineered DNA polymerase includes a 5′-exonuclease domain. In some embodiments, the substitution or substitution set of variants described herein set can be incorporated into the full length DNA polymerase that includes the DNA polymerase function and the 5′-exonuclease domain. In some embodiments, the engineered DNA polymerase described herein can be expressed as a fusion with the 5′-exonuclease domain of the DNA polymerase of Parageobacillus genomosp. 1. In some embodiments, the engineered DNA polymerase includes or is fused to a sequence corresponding to residues 1 to 284 of SEQ ID NO: 540, encoded by the polynucleotide sequence of SEQ ID NO: 539. Accordingly, for each and every engineered DNA polymerase described herein as a “large fragment,” the present disclosure also includes an engineered DNA polymerase that includes the 5′-exonuclease domain.

In some embodiments, the present disclosure further provides a large fragment of a DNA polymerase as provided in Table 10.2. In some embodiments, an engineered DNA polymerase comprises an amino acid sequence comprising:
amino acid residues 12-603 of SEQ ID NO: 490;
amino acid residues 12 to 605 of SEQ ID NO: 492;
amino acid residues 12-603 of SEQ ID NO: 494;
amino acid residues 12 to 603 of SEQ ID NO: 496;
amino acid residues 12-600 of SEQ ID NO: 498;
amino acid residues 12 to 603 of SEQ ID NO: 500;
amino acid residues 12-603 of SEQ ID NO: 502;
amino acid residues 12 to 605 of SEQ ID NO: 504;
amino acid residues 12-606 of SEQ ID NO: 506;
amino acid residues 12 to 503 of SEQ ID NO: 508;
amino acid residues 12-607 of SEQ ID NO: 510;
amino acid residues 12 to 604 of SEQ ID NO: 512;
amino acid residues 12-605 of SEQ ID NO: 514;
amino acid residues 12 to 600 of SEQ ID NO: 516;
amino acid residues 12-605 of SEQ ID NO: 518;
amino acid residues 12 to 601 of SEQ ID NO: 520;
amino acid residues 12-599 of SEQ ID NO: 522;
amino acid residues 12 to 599 of SEQ ID NO: 524;
amino acid residues 12-605 of SEQ ID NO: 526;
amino acid residues 12 to 600 of SEQ ID NO: 528;
amino acid residues 12-600 of SEQ ID NO: 530; or amino acid residues 12 to 602 of SEQ ID NO: 532.

In some embodiments, an engineered DNA polymerase comprises an amino acid sequence comprising SEQ ID NO: 2, 490, 492, 494, 496, 498, 500, 503, 504, 506, 508, 510, 512, 514, 516, 518, 520, 522, 524, 526, 528, 530, or 532.

In some embodiments, the present disclosure also provides an isolated DNA polymerase of a full length DNA polymerase provided in Table 10.2. In some embodiments, the DNA polymerase comprises the amino acid sequence of UniProt ID No. A0A023CMU9, P52026, A0A167UH07, M8D3Y0, A0A0A2SK72, Q08IE4, A0A4R1QH44, Q45458, A0A0B4SB30, A0A0D1JLC4, A0A0N0I8N0, A0A3R9UCK4, A0A084GX94, A0A176JAP1, A0A1W1II73, A0A2S0U8D5, A0A1I5VYY5, D5DMV6, E6U0L1, G8PDR9, K1KNJ5, L5N8Z2 or Q03RJ7, where the sequence for each UniProt ID No. are as of Sep. 12, 2022.

In some embodiments, the engineered DNA polymerase is in the form of a fusion protein. In some embodiments, the engineered DNA polymerase described herein can be fused to a variety of polypeptide sequences, such as, by way of example and not limitation, polypeptide tags that can be used for detection and/or purification. In some embodiments, the fusion protein of the engineered DNA polymerase comprises a glycine-histidine or histidine-tag (His-tag). In some embodiments, the fusion protein of the engineered DNA polymerase comprises an epitope tag, such as c-myc, FLAG, V5, or hemagglutinin (HA). In some embodiments, the fusion protein of the engineered DNA polymerase comprises a GST, SUMO, Strep, MBP, or GFP tag. In some embodiments, the fusion is to the amino (N-) terminus of engineered DNA polymerase polypeptide. In some embodiments, the fusion is to the carboxy (C-) terminus of the engineered DNA polymerase polypeptide.

In some embodiments, the present disclosure further provides functional fragments or biologically active fragments of engineered DNA polymerase polypeptides described herein. Thus, for each and every embodiment herein of an engineered DNA polymerase, a functional fragment or biologically active fragment of the engineered DNA polymerase is provided herewith. In some embodiments, a functional fragment or biologically active fragments of an engineered DNA polymerase comprises at least about 90%, 91%, 92%, 93%, 94%, 95%, 96%, 97%, 98%, or 99% of the activity of the DNA polymerase polypeptide from which it was derived (i.e., the parent DNA polymerase). In some embodiments, functional fragments or biologically active fragments comprise at least about 90%, 91%, 92%, 93%, 94%, 95%, 96%, 97%, 98%, or 99% of the parent sequence of the DNA polymerase. In some embodiments the functional fragment will be truncated by less than 5, less than 10, less than 15, less than 10, less than 25, less than 30, less than 35, less than 40, less than 45, and less than 50 amino acids.

In some embodiments, a functional fragment of an engineered DNA polymerase herein comprises at least 70%, 75%, 80%, 81%, 82%, 83%, 84%, 85%, 90%, 91%, 92%, 93%, 94%, 95%, 96%, 97%, 98%, or 99% of the parent sequence of the engineered DNA polymerase. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered DNA polymerase polypeptide described herein include at least a mutation or mutation set in the amino acid sequence of an engineered DNA polymerase described herein. Accordingly, in some embodiments, the functional fragments or biologically active fragments of the engineered DNA polymerase displays the enhanced or improved property associated with the mutation or mutation set in the parent DNA polymerase.

### Polynucleotides Encoding Engineered Polypeptides, Expression Vectors and Host Cells

In another aspect, the present disclosure provides recombinant polynucleotides encoding the engineered DNA polymerases described herein. In some embodiments, the recombinant polynucleotides are operably linked to one or more heterologous regulatory sequences that control gene expression to create a recombinant polynucleotide construct capable of expressing the DNA polymerase. In some embodiments, expression constructs containing at least one recombinant polynucleotide encoding the engineered DNA polymerase polypeptide(s) is introduced into appropriate host cells to express the corresponding DNA polymerase polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered DNA polymerase polypeptide of the present disclosure. Thus, the present disclosure provides methods and compositions for the production of each and every possible variation of engineered DNA polymerase polynucleotides that could be made that encode the engineered DNA polymerase polypeptides described herein by selecting combinations based on the possible codon choices, and all such polynucleotide variations are to be considered specifically disclosed for any DNA polymerase polypeptide described herein, including the amino acid sequences presented in the Examples (e.g., in Table 5.1, 6.1, 7.1, 8.1, 9.1, and 10.2) and in the Sequence Listing.

In some embodiments, the codons are preferably optimized for utilization by the chosen host cell for protein production. For example, preferred codons used in bacteria are typically used for expression in bacteria, and preferred codons used in mammalian cells are typically used for expression in mammalian cells. Consequently, codon optimized polynucleotides encoding the engineered DNA polymerase polypeptides contain preferred codons at about 40%, 50%, 60%, 70%, 80%, 90%, or greater than 90% of the codon positions in the full length coding region.

Accordingly, in some embodiments, a recombinant polynucleotide of the present disclosure comprises a polynucleotide sequence encoding an engineered DNA polymerase described herein. In some embodiments, the polynucleotide sequence of the recombinant polynucleotide is codon optimized.

In some embodiments, the recombinant polynucleotide comprises a polynucleotide sequence encoding an engineered DNA polymerase or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 2, 10, 80, 224, or 366, or to the reference sequence corresponding to SEQ ID NO: 2, 10, 80, 224, or 366, wherein the amino acid sequence comprises one or more substitutions relative to the reference sequence corresponding to amino acid residues 12 to 604 of SEQ ID NO: 2, 10, 80, 224, or 366, or relative to the reference sequence corresponding to SEQ ID NO: 2, 10, 80, 224, or 366, as described herein.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 2, or to the reference sequence corresponding to SEQ ID NO: 2, wherein the amino acid sequence comprises one or more substitutions relative to the reference sequence corresponding to amino acid residues 12 to 604 of SEQ ID NO: 2, or to the reference sequence corresponding to SEQ ID NO: 2.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of an even-numbered SEQ ID NO. of SEQ ID NOs: 4-488, or to the reference sequence corresponding to an even-numbered SEQ ID NO. of SEQ ID NOs: 4-488, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192, 210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution at amino acid position 36, 241, 372, or 470, or combinations thereof, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising a substitution or substitution set at amino acid position(s) 509, 300, 452, 36/241/372/470, 124/192/210/372/427/456/552, 124, 52, 483, 372, 393, 212, 52/66, 133, 454, 154, 593, 462, 541, 21, 573, 505, 152, 294, 545, 101/241/470, 584, 304, 295, 578, 456/470, 253, 290, 192/241/372/456, or 252, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution in an amino acid position set forth in Tables 5.1, 6.1, 7.1, 8.1, and 9.1, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least one substitution set forth in Tables 5.1, 6.1, 7.1, 8.1, and 9.1, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution or substitution set at the amino acid position(s) set forth in Tables 5.1, 6.1, 7.1, 8.1, and 9.1, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence comprising at least a substitution or substitution set forth for a variant set forth in Tables 5.1, 6.1, 7.1, 8.1, and 9.1, wherein the amino acid positions are relative to the reference sequence corresponding to SEQ ID NO: 2.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 604 of an even-numbered SEQ ID NO. of SEQ ID NOs: 4-488, or to a reference sequence corresponding to an even-numbered SEQ ID NO. of SEQ ID NOs: 4-488.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 4-488, or to the reference sequence corresponding to an even-numbered SEQ ID NO. of SEQ ID NOs: 4-488, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence comprising at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192, 210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence comprises at least a substitution at amino acid position 36, 241, 372, or 470, or combinations thereof, wherein the amino acid positions are relative to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence comprising at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 10, 80, 224, or 366.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, or to the reference sequence corresponding to SEQ ID NO: 10, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 10-218, or to the reference sequence corresponding to an even-numbered SEQ ID NO. of SEQ ID NOs: 10-218, wherein the amino acid sequence comprises one or more substitutions relative to the reference sequence corresponding to residues 12 to 604 of SEQ ID NO: 10, or relative to the reference sequence corresponding to SEQ ID NO: 10.

In some embodiments, the recombinant polynucleotide comprises a polynucleotide sequence encoding an engineered DNA polymerase comprising an amino acid sequence comprising at least a substitution set at amino acid position(s) 52/101/124/212/294/372/393/452/483/509, 52/124/300/393/452, 154/212/294/300/372/393/452/483/509, 52/101/154/294/300/452/509/593, 52/154/212/294/300/393/452, 212/300/393/452/509, 52/101/212/294/300/393/452/483/509/593, 52/124/294/300/452/509, 124/300/393/452/483/509/593, 101/124/212/452/483/509, 52/393/452/509/593, 212/300/452/509/593, 52/452/509/593, 154/212/300/372/452/509/593, 52/124/212/294/393/452/593, 124/452/509, 152/253/287/505/541/573/584, 154/212/300/452, 152/253/287/304/454/545/573/584, 52/124/154/294/300/372/452/509, 294/300/372/452/509/593, 52/212/452, 124/294/393/452, 52/300/509/593, 152/253/287/454/541/584, 52/101/452/509, 52/154/212/300/372/393/509, 21/253/287/454/584, 52/393/452, 52/124/154/294/509, 101/124/212/300/393, 124/294/300/372/452/509/593, 52/124/154/294/483/593, 52/372/393/452/509/593, 154/300/393/452/593, 52/101/294/372/393/452/509, 21/152/253/287/304/454/505, 52/300/393, 101/124/154/294/452/509, 154/212/372/393/509/593, 21/253/287/454/505/541, 287/454/505/584, 52/124/294/300/372/452, 287/505/541/573, 253/505/584, 84/253/287/456/573, 52/372/509/593, 509/593, 154/212/300/372/509, 52/154/294/509/593, 152/295/505/545/573, 253/541/584, 253/295/304/454/462/573/584, 253/454/505/573/584, 154/212/300, 287/454/545/573, 21/152/287/304/454, 152/253/295/304/541/573, 152/253/287/304/454, 152/454/573, 124/294/372/509/593, 152/253/295/454, 154/300/452, 287/304/505, 152/253/454/505, 253/454/505, 21/253/454/505/545, 152/253/287/505, 505/541/584, or 152/253/454/584, wherein the amino acid positions are relative to the reference sequence corresponding to SEQ ID NO: 10.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 80, or to the reference sequence corresponding to SEQ ID NO: 80, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 220-258, or to the reference sequence corresponding to an even-numbered SEQ ID NO. of SEQ ID NOs: 220-258, wherein the amino acid sequence comprises one or more substitutions relative to the reference sequence corresponding to residues 12 to 604 of SEQ ID NO: 80, or relative to the reference sequence corresponding to SEQ ID NO: 80.

In some embodiments, the recombinant polynucleotide comprises a polynucleotide sequence encoding the engineered DNA polymerase comprising an amino acid sequence comprising at least a substitution or substitution set at amino acid position(s) 300/454/456/541/584, 21/300/454/545/584, 253/300/454/456/584, 154/253/300/454/456/505/573, 21/154/300/454/456/573, 152/300/454/456/505/584/593, 154/253/300/456/505/545/573/584, 253/300/454/456/505/573, 154/253/300/454/456/541/573/584, 154/300/454/505, 21/253/300/454/456/545/573/593, 168/300/454/456/545/573, 253/300/454/505/573, 154/300/505/545/584, 154/253/300/456/541/573/584, 21/154/300/454/456/505/545, 154/253/300/454/505, 21/154/253/300/454/573, 300/454/505/541/584, 300/454/505/541/573, 154/253/300/454, 154/300/454/573, 300/454/456/545/573, 253/300/454/456, 154/253/300/454/545/573, 21/152/253/300/454/456/541/573, 154/253/300/454/456/573, 152/300/454, 154/253/300/454/505/541/593, 21/154/300/454/573/584, 154/300/456/573/593, 152/253/300/454/545/573, 21/300/456/495/505/541/584, 154/300/454/541/584, 253/300/505/541/573/584, 253/300/584, 253/300/454/456/545, 21/253/300/573, 300/454/545, 454/456/541/545/573/584, 152/253/454/505/545/573, 21/300/454/456/495, 300/573, 154/300/454/545, 154/253/300/545/584, 21/300/505, 21/152/253/300/454/456/545, 154/253/300/456/541/573, 154/300/454, 253/300/454/541, 253/454/456/541/584, 21/300/573, 300/454/456, 300/454, 154/253/300/454/456, 154/456/505/593, 454/456/505, 253/454/456/584, 154/253/300, 253/456/505/584, 454/456, 154/454, 584, 432/505/573, 456/584, 21/505/545/584, 21/154/545/573, 454/584, 154/253/505, or 505, wherein the amino acid positions are relative to the reference sequence corresponding to SEQ ID NO: 80.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 224, or to the reference sequence corresponding to SEQ ID NO: 224, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 360-400, or to the reference sequence corresponding to an even-numbered SEQ ID NO. of SEQ ID NOs: 360-400, wherein the amino acid sequence comprises one or more substitutions relative to the reference sequence corresponding to residues 12 to 604 of SEQ ID NO: 224, or relative to the reference sequence corresponding to SEQ ID NO: 224.

In some embodiments, the recombinant polynucleotide comprises a polynucleotide sequence encoding an engineered DNA polymerase comprising an amino acid sequence comprising at least a substitution set at amino acid positions 144/154/505/547/573/584, 154/191/325/505/573/584, 144/154/373/374/505/573/584, 134/136/154/505/547/573/584, 81/144/154/505/547/573/584, 154/505/573/584, 144/154/191/230/322/505/573/584, 68/144/154/505/573/584, 144/154/226/230/505/573/584, 144/154/374/486/505/573/584, 81/114/144/154/505/573/584, 144/154/276/505/573/584, 134/144/154/505/573/584, 144/154/505/573/584, 81/144/154/183/505/547/573/584, 68/81/133/134/144/154/505/547/573/584, 144/154/374/505/573/584, 92/133/144/154/505/573/584, 144/154/373/505/573/584, 114/144/154/505/573/584, 154/505/551/573/584/585, or 81/154/505/547/573/584, wherein the amino acid positions are relative to the reference sequence corresponding to SEQ ID NO: 224.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 366, or to the reference sequence corresponding to SEQ ID NO: 366, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 402-488, or to the reference sequence corresponding to an even-numbered SEQ ID NO. of SEQ ID NOs: 402-488, wherein the amino acid sequence comprises one or more substitutions relative to the reference sequence corresponding to residues 12 to 604 of SEQ ID NO: 366, or relative to the reference sequence corresponding to SEQ ID NO: 366.

In some embodiments, the recombinant polynucleotide comprises a polynucleotide sequence encoding an engineered DNA polymerase comprising an amino acid sequence comprising at least a substitution or substitution set at amino acid position(s) 24/58/432, 58/432/575, 58/115/432/575, 24/115/432/575, 24/25/221/432, 25/58/432/575, 24/25/58/432/575, 221/432/575, 432/575, 24/221/432, 24/115/221/432, 24/115/221/432/575, 432, 24/34/432, 24/432/575, 184/221/432/575, 24/432, 24/25/58/115/432/575, 25/221/432, 25/58/115/432, 221/575, 221/432, 24/58/115/221/575, 24/25/221/432/575, 24/25/58/221/432, 34/58/105/432, 24/25/115/432, 24/221/575, 115/432, 58/221, 58/575, 25/432/575, 25/432, 24/221/432/575, 24/115/432, 24/25/105/432, 25/34/432, 25/105/221/432, 24/25/432, 24/58/575, or 24/25/105/432/575, wherein the amino acid positions are relative to the reference sequence corresponding to SEQ ID NO: 366.

In some embodiments, the recombinant polynucleotide comprises a polynucleotide sequence encoding an engineered DNA polymerase comprising an amino acid sequence comprising at least a substitution in an amino acid position provided in Tables 6.1, 7.1, 8.1, and 9.1, wherein the positions are relative to the reference sequence corresponding to SEQ ID NO: 2, 10, 80, 224, or 366, as provided in each of the Tables.

In some embodiments, the recombinant polynucleotide comprises a polynucleotide sequence encoding an engineered DNA polymerase comprising an amino acid sequence comprising at least one substitution provided in Tables 6.1, 7.1, 8.1, and 9.1, wherein the amino acid positions of the substitutions are relative to the reference sequence corresponding to SEQ ID NO: 2, 10, 80, 224, or 366, as provided in each of the Tables.

In some embodiments, the recombinant polynucleotide comprises a polynucleotide sequence encoding an engineered DNA polymerase comprising an amino acid sequence comprising at least a substitution or substitution set at amino acid position(s) provided in Tables 6.1, 7.1, 8.1, and 9.1, wherein the amino acid positions are relative to the reference sequence corresponding to SEQ ID NO: 2, 10, 80, 224, or 366, as provided in each of the Tables.

In some embodiments, the recombinant polynucleotide comprises a polynucleotide sequence encoding an engineered DNA polymerase comprising an amino acid sequence comprising at least a substitution or substitution set of a variant set forth in Tables 6.1, 7.1, 8.1, and 9.1, wherein the amino acid positions of the substitution or substitution set are relative to the reference sequence corresponding to SEQ ID NO: 2, 10, 80, 224, or 366, as provided in each of the Tables.

In some embodiments, the recombinant polynucleotide comprises a polynucleotide sequence encoding an engineered DNA polymerase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered DNA polymerase variant set forth in Tables 5.1, 6.1, 7.1, 8.1, and 9.1.

In some embodiments, the recombinant polynucleotide comprises a polynucleotide sequence encoding an engineered DNA polymerase comprising an amino acid sequence comprising residues 12 to 604 of an even-numbered SEQ ID NO. of SEQ ID NOs: 4-488, or comprising an even-numbered SEQ ID NO. of SEQ ID NOs: 4-488.

In some embodiments, the recombinant polynucleotide comprises a polynucleotide sequence encoding an engineered DNA polymerase comprising an amino acid sequence comprising residues 12 to 604 of SEQ ID NO: 10, 80, 224, or 366, or comprising SEQ ID NO: 10, 80, 224, or 366.

In some embodiments, the recombinant polynucleotide comprises a polynucleotide sequence encoding an engineered DNA polymerase comprising an amino acid sequence comprising residues 12 to 604 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 440, 442, 444, 446, 448, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, or 488, optionally wherein the engineered DNA polymerase has 1, 2, 3, 4, 5, 6, 7, 8, 9, or up to 10 substitutions in the amino acid sequence.

In some embodiments, the recombinant polynucleotide comprises a polynucleotide sequence encoding an engineered DNA polypeptide comprising an amino acid sequence comprising SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 266, 268, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 440, 442, 444, 446, 448, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, or 488, optionally wherein the engineered DNA polymerase has 1, 2, 3, 4, 5, 6, 7, 8, 9, or up to 10 substitutions in the amino acid sequence.

In some embodiments, the encoded engineered DNA polymerase polypeptide includes 1, 2, 3, 4, up to 5 substitutions in the amino acid sequence. In some embodiments, the engineered DNA polymerase polypeptide includes 1, 2, 3, or 4 substitutions in the amino acid sequence. In some embodiments, the substitutions comprises conservative substitutions. In some embodiments, the substitutions comprises non-conservative substitutions. In some embodiments, the substitutions comprises non-conservative and conservative substitutions.

In some embodiments, the recombinant polynucleotide comprises a polynucleotide sequence encoding an engineered DNA polymerase comprising an amino acid sequence comprising residues 12 to 604 of SEQ ID NO: 10, 80, 224, or 366, or an amino acid sequence comprising SEQ ID NO: 10, 80, 224, or 366, optionally wherein the amino acid sequence has 1, 2, 3, 4, 5, 6, 7, 8, 9, or up to 10 substitutions in the polypeptide sequence. In some embodiments, the encoded DNA polymerase includes 1, 2, 3, 4, up to 5 substitutions in the amino acid sequence. In some embodiments, the encoded DNA polymerase includes 1, 2, 3, or 4 substitutions in the amino acid sequence.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1812 of SEQ ID NO: 1, 9, 79, 223, or 365, or to a reference polynucleotide sequence corresponding to SEQ ID NO: 1, 9, 79, 223, or 365, wherein the recombinant polynucleotide encodes an engineered DNA polymerase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34 to 1812 of an odd-numbered SEQ ID NO. of SEQ ID NOs: 3-487, or to a reference polynucleotide sequence corresponding to an odd-numbered SEQ ID NO. of SEQ ID NOs: 3-487, wherein the recombinant polynucleotide encodes an engineered DNA polymerase.

In some embodiments, the recombinant polynucleotide encoding an engineered DNA polymerase comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a polynucleotide sequence corresponding to nucleotide residues 34 to 1812 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, or 487.

In some embodiments, the recombinant polynucleotide encoding an engineered DNA polymerase comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a polynucleotide sequence corresponding to SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, or 487.

In some embodiments, the recombinant polynucleotide encoding an engineered DNA polymerase comprises a polynucleotide sequence comprising nucleotide residues 34 to 1812 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, or 487.

In some embodiments, the recombinant polynucleotide encoding an engineered DNA polymerase comprises a polynucleotide sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, or 487.

In some embodiments, the recombinant polynucleotide comprises a polynucleotide sequence comprising nucleotide residues 34 to 1812 of SEQ ID NO: 9, 79, 223, or 365, or a polynucleotide sequence comprising SEQ ID NO: 9, 79, 223, or 365.

In some embodiments, the recombinant polynucleotide encodes a DNA polymerase comprising an amino acid sequence comprising:
amino acid residues 12 to 603 of SEQ ID NO: 490;
amino acid residues 12 to 605 of SEQ ID NO: 492;
amino acid residues 12 to 603 of SEQ ID NO: 494;
amino acid residues 12 to 603 of SEQ ID NO: 496;
amino acid residues 12 to 600 of SEQ ID NO: 498;
amino acid residues 12 to 603 of SEQ ID NO: 500;
amino acid residues 12 to 603 of SEQ ID NO: 502;
amino acid residues 12 to 605 of SEQ ID NO: 504;
amino acid residues 12 to 606 of SEQ ID NO: 506;
amino acid residues 12 to 503 of SEQ ID NO: 508;
amino acid residues 12 to 607 of SEQ ID NO: 510;
amino acid residues 12 to 604 of SEQ ID NO: 512;
amino acid residues 12 to 605 of SEQ ID NO: 514;
amino acid residues 12 to 600 of SEQ ID NO: 516;
amino acid residues 12 to 605 of SEQ ID NO: 518;
amino acid residues 12 to 601 of SEQ ID NO: 520;
amino acid residues 12 to 599 of SEQ ID NO: 522;
amino acid residues 12 to 599 of SEQ ID NO: 524;
amino acid residues 12 to 605 of SEQ ID NO: 526;
amino acid residues 12 to 600 of SEQ ID NO: 528;
amino acid residues 12 to 600 of SEQ ID NO: 530; or
amino acid residues 12 to 602 of SEQ ID NO: 532.

In some embodiments, the recombinant polynucleotide encodes an engineered DNA polymerase comprising an amino acid sequence comprising SEQ ID NO: 2, 490, 492, 494, 496, 498, 500, 502, 504, 506, 508, 510, 512, 514, 516, 518, 520, 522, 524, 526, 528, 530, or 532.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to SEQ ID NO: 1, 505, 507, 509, 511, 513, 515, 517, 519, 521, 523, 525, 527, 529, or 531, wherein the recombinant polynucleotide encodes a DNA polymerase, as described herein. In some embodiments, the recombinant polynucleotide encoding an engineered DNA polymerases comprises a polynucleotide sequence comprising SEQ ID NO: 1, 489, 491, 493, 495, 497, 499, 501, 503, 505, 507, 509, 511, 513, 515, 517, 519, 521, 523, 525, 527, 529, or 531.

In some embodiments, the recombinant polynucleotide hybridizes under highly stringent conditions to a reference polynucleotide sequence described herein encoding an engineered DNA polymerase, or a reverse complement thereof. In some embodiments, the reference polynucleotide sequence corresponds to residues 34 to 1812 of SEQ ID NO: 1, 9, 79, 223, or 365, or to the sequence corresponding to SEQ ID NO: 1, 9, 79, 223, or 365, or a reverse complement thereof, or a polynucleotide sequence encoding any of the other engineered DNA polymerases provided herein, or a reverse complement thereof. In some embodiments, the recombinant polynucleotide hybridizes under highly stringent conditions to a reference polynucleotide sequence corresponding to residues 34 to 1812 of an odd numbered SEQ ID NO. of SEQ ID NOs: 1-487, or to a reference polynucleotide comprising a sequence corresponding to an odd numbered SEQ ID NO. of SEQ ID NOs: 1-487, or to a reverse complement thereof.

In some embodiments, the recombinant polynucleotide hybridizing under highly stringent conditions comprises a sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to at least one reference polynucleotide sequence corresponding to residues 34 to 1812 of a polynucleotide sequence set forth in Tables 5.1, 6.1, 7.1, 8.1, or 9.1, or a polynucleotide sequence set forth in Tables 5.1, 6.1, 7.1, 8.1, or 9.1.

In some embodiments, a recombinant polynucleotide encoding any of the DNA polymerases herein is manipulated in a variety of ways to facilitate expression of the DNA polymerase polypeptide. In some embodiments, the recombinant polynucleotide encoding the DNA polymerase comprises an expression vector where one or more control sequences is present to regulate the expression of the DNA polymerase polynucleotides and/or polypeptides. Manipulation of the isolated polynucleotide prior to its insertion into a vector may be desirable or necessary depending on the expression vector utilized. Techniques for modifying polynucleotides and nucleic acid sequences utilizing recombinant DNA methods are well known in the art. In some embodiments, the control sequences include among others, promoters, leader sequences, polyadenylation sequences, propeptide sequences, signal peptide sequences, and transcription terminators.

In some embodiments, suitable promoters are selected based on the host cell used. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (see, e.g., Villa-Kamaroff et al., Proc. Natl Acad. Sci. USA, 1978, 75:3727-3731), as well as the tac promoter (see, e.g., DeBoer et al., Proc. Natl Acad. Sci. USA, 1983, 80:21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (see, e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (see, e.g., Romanos et al., Yeast, 1992, 8:423-488). Exemplary promoters for use in mammalian cells include, but are not limited to, those from cytomegalovirus (CMV), chicken β-actin promoter fused with the CMV enhancer, Simian vacuolating virus 40 (SV40), from Homo sapiens genes for phosphoglycerate kinase, beta actin, elongation factor-1a or glyceraldehyde-3-phosphate dehydrogenase, or from Gallus D-actin.

In some embodiments, the control sequence is a suitable transcription terminator sequence (i.e., a sequence recognized by a host cell to terminate transcription). In some embodiments, the terminator sequence is operably linked to the 3′ terminus of the nucleic acid sequence encoding the DNA polymerase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5):793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (see, e.g., Romanos et al., supra). Exemplary terminators for mammalian cells include, but are not limited to those from cytomegalovirus (CMV), Simian virus 40 (SV40), from Homo sapien genes for growth hormone hGH, bovine growth hormone BGH, and human or rabbit beta globulin.

In some embodiments, the control sequence is a suitable leader sequence, a non-translated region of an mRNA that is important for translation by the host cell. In some embodiments, the leader sequence is operably linked to the 5′ terminus of the nucleic acid sequence encoding the DNA polymerase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA or SV40 mRNA.

In some embodiments, the control sequence is a polyadenylation sequence (i.e., a sequence operably linked to the 3′ terminus of the nucleic acid sequence and which, when transcribed, is recognized by the host cell as a signal to add polyadenosine residues to transcribed mRNA). Any suitable polyadenylation sequence which is functional in the host cell of choice finds use in the present invention. Exemplary polyadenylation sequences for filamentous fungal host cells include, but are not limited to the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are known (see, e.g., Guo and Sherman, Mol. Cell. Biol., 1995, 15:5983-5990). Useful polyadenylation and 3′ UTR sequences for mammalian host cells include, but are not limited to the 3′-UTRs of α- and β-globin mRNAs that harbor several sequence elements that increase the stability and translation of mRNA, as well as those from mammalian viruses, such as SV40.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence contains a signal peptide coding region naturally linked in translation reading frame with the segment of the coding region that encodes the secreted polypeptide. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (see, e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to, those from the genes for immunoglobulin gamma (IgG) and the signal peptide in a human secreted protein, such as human beta-galactosidase polypeptide.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen.” A propolypeptide can be converted to a mature active polypeptide by catalytic or autocatalytic cleavage of the propeptide from the propolypeptide. The propeptide coding region may be obtained from any suitable source, including, but not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (see, e.g., WO95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, the control sequence is a regulatory sequence that facilitates regulation of the expression of the recombinant polynucleotide and/or encoded polypeptide. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present disclosure provides a recombinant expression vector comprising a recombinant polynucleotide encoding an engineered DNA polymerase polypeptide, where the recombinant polynucleotide is operably linked to one or more control sequences, such as a promoter and a terminator, a replication origin, etc., depending on the type of hosts into which they are to be introduced, e.g., for expression of the polynucleotide and/or encoded polypeptide. In some embodiments, the various nucleic acid and control sequences described herein are joined together (i.e., operably linked) to the recombinant polynucleotide produce recombinant expression vectors which include one or more convenient restriction sites to allow for insertion or substitution of the nucleic acid sequence encoding the DNA polymerase polypeptide at such sites. In some embodiments, the recombinant expression vector may be any suitable vector (e.g., a plasmid or virus), that can be conveniently subjected to recombinant DNA procedures and bring about the expression of the DNA polymerase polynucleotide and/or encoded polypeptide. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector is one in which, when introduced into the host cell, it is integrated into the genome and replicated together with the chromosome(s) into which it has been integrated. Furthermore, in some embodiments, a single vector or plasmid, or two or more vectors or plasmids which together contain the total DNA to be introduced into the genome of the host cell, and/or a transposon is utilized.

In some embodiment, recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising a polynucleotide encoding at least one engineered DNA polymerase polypeptide of the present disclosure, the polynucleotide(s) being operably linked to one or more control sequences for expression of the engineered DNA polymerase enzyme(s) in the host cell. In some embodiments, the host cell comprises an expression vector comprising a recombinant polynucleotide encoding an engineered DNA polymerase polypeptide described herein. In some embodiments, the host cells suitable for use in expressing the polypeptides encoded by the expression vectors is a prokaryotic cell or eukaryotic cell. Host cells known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal (e.g., mammalian) cells such as CHO, COS, BHK, 293, and Bowes melanoma cells; and plant cells. Exemplary host cells also include various Escherichia coli strains (e.g., W3110 (AfhuA) and BL21).

In another aspect, the present disclosure provides a method of producing the engineered DNA polymerase polypeptides, where the method comprises culturing a host cell capable of expressing a polynucleotide encoding the engineered DNA polymerase polypeptide under conditions suitable for expression of the polypeptide such that the engineered DNA polymerase is produced. In some embodiments, the method further comprises isolating the engineered DNA polymerase from the culture media and/or host cells. In some embodiments, the method further comprises purifying the DNA polymerase polypeptides, such as by the methods described herein.

Appropriate culture media and growth conditions for host cells are known in the art. It is contemplated that any suitable method for introducing polynucleotides for expression of the DNA polymerase polypeptides into host cells will find use in the present invention. Suitable techniques include, but are not limited to electroporation, biolistic particle bombardment, liposome mediated transfection, calcium chloride transfection, and protoplast fusion.

In some embodiments, the recombinant polypeptides (e.g., DNA polymerase enzyme variants) can be produced using any suitable methods known the art. For example, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (region-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased stability, increased processivity, increased fidelity, increased inhibitor resistance or tolerance, increased salt tolerance, and/or pH stability, etc.).

In some embodiments, the engineered DNA polymerase polypeptides with the properties disclosed herein can be obtained by subjecting the polynucleotide encoding the naturally occurring or engineered DNA polymerase polypeptide to a suitable mutagenesis and/or directed evolution methods known in the art, for example, as described herein. An exemplary directed evolution technique is mutagenesis and/or DNA shuffling (see, e.g., Stemmer, Proc. Natl. Acad. Sci. USA, 1994, 91:10747-10751; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (see, e.g., Zhao et al., Nat. Biotechnol., 1998, 16:258-261), mutagenic PCR (see, e.g., Caldwell et al., PCR Methods Appl., 1994, 3:S136-S140), and cassette mutagenesis (see, e.g., Black et al., Proc. Natl. Acad. Sci. USA, 1996, 93:3525-3529).

Mutagenesis and directed evolution methods can be applied to DNA polymerase-encoding polynucleotides to generate variant libraries that can be expressed, screened, and assayed. Any suitable mutagenesis and directed evolution methods find use in the present disclosure (see, e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, 9,665,694, 9,684,771, and all related PCT and non-US counterparts; Ling et al., Anal. Biochem., 1997, 254(2):157-78; Dale et al., Meth. Mol. Biol., 1996, 57:369-74; Smith, Ann. Rev. Genet., 1985, 19:423-462; Botstein et al., Science, 1985, 229:1193-1201; Carter, Biochem. J., 1986, 237:1-7; Kramer et al., Cell, 1984, 38:879-887; Wells et al., Gene, 1985, 34:315-323; Minshull et al., Curr. Op. Chem. Biol., 1999, 3:284-290; Christians et al., Nat. Biotechnol., 1999, 17:259-264; Crameri et al., Nature, 1998, 391:288-291; Crameri, et al., Nat. Biotechnol., 1997, 15:436-438; Zhang et al., Proc. Nat. Acad. Sci. U.S.A., 1997, 94:4504-4509; Crameri et al., Nat. Biotechnol., 1996, 14:315-319; Stemmer, Nature, 1994, 366:389-391; Stemmer, Proc. Nat. Acad. Sci. USA, 1994, 91:10747-10751; EP 3 049 973; WO 95/22625; WO 97/0078; WO 97/35966; WO 98/27230; WO 00/42651; WO 01/75767; WO 2009/152336; and WO 2015/048573, all of which are incorporated herein by reference).

In some embodiments, the clones obtained following mutagenesis treatment are screened by subjecting the enzyme preparations to a defined treatment conditions or assay conditions (e.g., temperature, pH condition, type of template (e.g., DNA, RNA, GC content, secondary structure, etc.), input template concentration, nucleotides, etc.) and measuring enzyme activity after the treatments or other suitable assay conditions. Clones containing a polynucleotide encoding the polypeptide of interest are then isolated from the gene, sequenced to identify the nucleotide sequence changes (if any), and used to express the enzyme in a host cell. Measuring enzyme activity from the expression libraries can be performed using any suitable method known in the art and as described in the Examples.

For engineered polypeptides of known sequence, the polynucleotides encoding the enzyme can be prepared by standard solid-phase methods, according to known synthetic methods. In some embodiments, nucleic acid fragments of up to about 100 bases can be individually synthesized, then joined (e.g., by enzymatic or chemical ligation methods, or polymerase mediated methods) to form any desired continuous sequence. For example, polynucleotides and oligonucleotides disclosed herein can be prepared by chemical synthesis using the classical phosphoramidite method (see, e.g., Beaucage et al., Tet. Lett., 1981, 22:1859-69; and Matthes et al., EMBO J., 1984, 3:801-05), as it is typically practiced in automated synthetic methods.

In some embodiments, a method for preparing the engineered DNA polymerase polypeptide can comprise: (a) synthesizing a polynucleotide encoding a polypeptide comprising an amino acid sequence selected from any variant as described herein, and (b) expressing the DNA polymerase polypeptide encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7,1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions are conservative or non-conservative substitutions.

In some embodiments, any of the engineered DNA polymerase polypeptides expressed in a host cell are recovered and/or purified from the cells and/or the culture medium using any one or more of the known techniques for protein purification, including, among others, lysozyme treatment, sonication, filtration, salting-out, ultra-centrifugation, and chromatography.

Chromatographic techniques for isolation and purification of the DNA polymerase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying a particular enzyme may depend, in part, on factors such as net charge, hydrophobicity, hydrophilicity, molecular weight, molecular shape, etc., and will be apparent to those having skill in the art. In some embodiments, affinity techniques may be used to isolate the improved DNA polymerase enzymes. For affinity chromatography purification, any antibody that specifically binds a DNA polymerase polypeptide of interest can be used. For the production of antibodies, various host animals, including but not limited to rabbits, mice, rats, etc., are immunized by injection with a DNA polymerase polypeptide, or a fragment thereof. In some embodiments, the DNA polymerase polypeptide or fragment is attached to a suitable carrier, such as BSA, by means of a side chain functional group or linkers attached to a side chain functional group. Where the engineered DNA polymerase includes a fusion polypeptide that allows for affinity purification, such as a His-tag, standard affinity methods for the particular fusion protein can be used.

### Compositions of DNA Polymerase

In a further aspect, the present disclosure provides compositions of the DNA polymerases disclosed herein. In some embodiments, the composition comprises at least one engineered DNA polymerase polypeptide described herein. In some embodiments, the engineered DNA polymerase polypeptide in the compositions is isolated or purified. In some embodiments, the DNA polymerase is combined with other components and compounds to provide compositions and formulations comprising the engineered DNA polymerase polypeptide as appropriate for different applications and uses (e.g., diagnostic methods, molecular biological tools, and compositions).

In some embodiments, a composition comprises a DNA polymerase comprising an amino acid sequence comprising residues 12 to 604 of SEQ ID NO: 2, or a sequence comprising SEQ ID NO: 2, and/or at least one engineered DNA polymerase described herein. In some embodiments, the compositions comprises an engineered DNA polymerase comprising an amino acid sequence comprising residues 12 to 604 of an even-numbered SEQ ID NO. of SEQ ID NOs: 4-488, or an amino acid sequence comprising an even-numbered SEQ ID NO. of SEQ ID NOs: 4-488,

In some embodiments, a composition comprises a large fragment of a DNA polymerase provided in Table 10.2. In some embodiments, the composition comprises a DNA polymerase comprising an amino acid sequence comprising:
amino acid residues 12-603 of SEQ ID NO: 490;
amino acid residues 12-605 of SEQ ID NO: 492;
amino acid residues 12-603 of SEQ ID NO: 494;
amino acid residues 12-603 of SEQ ID NO: 496;
amino acid residues 12-600 of SEQ ID NO: 498;
amino acid residues 12-603 of SEQ ID NO: 500;
amino acid residues 12-603 of SEQ ID NO: 502;
amino acid residues 12-605 of SEQ ID NO: 504;
amino acid residues 12-606 of SEQ ID NO: 506;
amino acid residues 12-503 of SEQ ID NO: 508;
amino acid residues 12-607 of SEQ ID NO: 510;
amino acid residues 12 to 604 of SEQ ID NO: 512;
amino acid residues 12-605 of SEQ ID NO: 514;
amino acid residues 12-600 of SEQ ID NO: 516;
amino acid residues 12-605 of SEQ ID NO: 518;
amino acid residues 12-601 of SEQ ID NO: 520;
amino acid residues 12-599 of SEQ ID NO: 522;
amino acid residues 12-599 of SEQ ID NO: 524;
amino acid residues 12-605 of SEQ ID NO: 526;
amino acid residues 12-600 of SEQ ID NO: 528;
amino acid residues 12-600 of SEQ ID NO: 530; or
amino acid residues 12-602 of SEQ ID NO: 532.

In some embodiments, the composition comprises a DNA polymerase comprising an amino acid sequence comprising SEQ ID NO: 490, 492, 494, 496, 498, 500, 502, 504, 506, 508, 510, 512, 514, 516, 518, 520, 522, 524, 526, 528, 530, or 532.

In some embodiments, a composition comprises a full length DNA polymerase provided in Table 10.2. In some embodiments, the composition comprises a DNA polymerase having the amino acid sequence of UniProt ID No. A0A023CMU9, P52026, A0A167UH07, M8D3Y0, A0A0A2SK72, Q08IE4, A0A4R1QH44, Q45458, A0A0B4SB30, A0A0D1JLC4, A0A0N0I8N0, A0A3R9UCK4, A0A084GX94, A0A176JAP1, A0A1W1II73, A0A2S0U8D5, A0A1I5VYY5, D5DMV6, E6U0L1, G8PDR9, K1KNJ5, L5N8Z2 or Q03RJ7.

In some embodiments, the composition further comprises one or more of a buffer, a nucleotide substrate (e.g., dNTPs, dNTP analogs, and/or modified dNTPs), and/or at least one primer, e.g., synthetic primer complementary to a target nucleic acid. In some embodiments, the composition further comprises a template polynucleotide, particularly a template DNA or RNA. In some embodiments, the template polynucleotide comprises a heterologous template DNA or RNA. In some embodiments, the template polynucleotide comprises a mixture of DNA and RNA.

In some embodiments, the composition can further comprise a DNA polymerase (e.g., a second DNA polymerase) other than the engineered DNA polymerase described herein. In some embodiments, the second DNA polymerase is a second thermostable DNA polymerase, for example Taq or Pfu polymerase, or a reverse transcriptase, such as those useful in RT-PCR coupled reactions. In some embodiments, the composition includes a probe or indicator, such as a nucleic acid binding dye (e.g., SYBR® Green, EvaGreen®, etc.), for detecting and/or quantitating the amount of product formed, e.g., in a qRT-PCR reaction.

In some embodiments, an engineered DNA polymerase described herein is provided in solution or is immobilized on a substrate. In some embodiments, the substrate is a solid substrate or a membrane or particles. The enzyme can be entrapped in matrixes or membranes. In some embodiments, matrices include polymeric materials such as calcium-alginate, agar, k-carrageenin, polyacrylamide, and collagen, or solid matrices, such as activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the enzyme is immobilized on the surface of a support material. In some embodiments, the enzyme is adsorbed on the support material. In some embodiments, the enzyme is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

### Uses of Engineered DNA Polymerase Polypeptides and Kits

In another aspect, the present disclosure provides uses of the engineered DNA polymerases for diagnostic and molecular biological purposes, such as for detecting the presence of a target nucleic acid, nucleic acid sequencing, and direct/indirect amplification of nucleic acids.

In some embodiments, the engineered DNA polymerase is used in preparing a complementary DNA of a target nucleic acid/polynucleotide. In some embodiments, a method of preparing a complementary DNA of a target nucleic acid/polynucleotide comprises contacting a target nucleic acid/polynucleotide with a DNA polymerase having a sequence comprising residues 12 to 604 of SEQ ID NO: 2, or an amino acid sequence comprising SEQ ID NO: 2; an engineered DNA polymerase described herein; or a DNA polymerase described in Table 10.2, in presence of substrates sufficient for producing a complementary DNA under reaction conditions suitable for production of a complementary DNA to all or a portion (i.e., whole or in part) of the target nucleic acid/polynucleotide. In some embodiments, the target nucleic acid/polynucleotide is DNA. In some embodiments, the target nucleic acid/polynucleotide is RNA. In some embodiments, the target nucleic acid/polynucleotide comprises a mixture of DNA and RNA.

In some embodiments, the DNA polymerase for use in the methods comprises an amino acid sequence comprising:
amino acid residues 12 to 603 of SEQ ID NO: 490;
amino acid residues 12 to 605 of SEQ ID NO: 492;
amino acid residues 12 to 603 of SEQ ID NO: 494;
amino acid residues 12 to 603 of SEQ ID NO: 496;
amino acid residues 12 to 600 of SEQ ID NO: 498;
amino acid residues 12 to 603 of SEQ ID NO: 500;
amino acid residues 12 to 603 of SEQ ID NO: 502;
amino acid residues 12 to 605 of SEQ ID NO: 504;
amino acid residues 12 to 606 of SEQ ID NO: 506;
amino acid residues 12 to 503 of SEQ ID NO: 508;
amino acid residues 12 to 607 of SEQ ID NO: 510;
amino acid residues 12 to 604 of SEQ ID NO: 512;
amino acid residues 12 to 605 of SEQ ID NO: 514;
amino acid residues 12 to 600 of SEQ ID NO: 516;
amino acid residues 12 to 605 of SEQ ID NO: 518;
amino acid residues 12 to 601 of SEQ ID NO: 520;
amino acid residues 12 to 599 of SEQ ID NO: 522;
amino acid residues 12 to 599 of SEQ ID NO: 524;
amino acid residues 12 to 605 of SEQ ID NO: 526;
amino acid residues 12 to 600 of SEQ ID NO: 528;
amino acid residues 12 to 600 of SEQ ID NO: 530; or
amino acid residues 12 to 602 of SEQ ID NO: 532.

In some embodiments, the DNA polymerase for use in the methods comprises an amino acid sequence comprising SEQ ID NO: 490, 492, 494, 496, 498, 500, 502, 504, 506, 508, 510, 512, 514, 516, 518, 520, 522, 524, 526, 528, 530, or 532.

In some embodiments, the DNA polymerase for use in the methods herein comprises a full length DNA polymerase provided in Table 10.2. In some embodiments, the DNA polymerase for use in the methods comprises an amino acid sequence of UniProt ID No. A0A023CMU9, P52026, A0A167UH07, M8D3Y0, A0A0A2SK72, Q08IE4, A0A4R1QH44, Q45458, A0A0B4SB30, A0A0D1JLC4, A0A0N0I8NO, A0A3R9UCK4, A0A084GX94, A0A176JAP1, A0A1W1II73, A0A2S0U8D5, A0A1I5VYY5, D5DMV6, E6U0L1, G8PDR9, K1KNJ5, L5N8Z2 or Q03RJ7.

As discussed herein and known in the art, substrates include nucleotides (e.g., dNTPs or dNTP analogs) for DNA polymerase activity and/or oligonucleotide primers. Primers can be complementary to one or more of a specific sequence of the target nucleic acid, or random primers, such as for generation of DNA libraries. In some embodiments, the reaction conditions include components needed for DNA polymerase activity, such as a divalent metal, e.g., Mg⁺², and/or a buffer at an appropriate pH.

In some embodiments, the target nucleic acid/polynucleotide is any DNA or RNA appropriate as a template for the engineered DNA polymerase, including, but not limited to, genomic DNA or mRNA, mitochondrial DNA or RNA, cell-free DNA or cell-free RNA (e.g., obtained from blood/serum), bacterial DNA or RNA, fungal DNA or RNA, plant DNA or RNA, mammalian DNA or RNA, or viral DNA or RNA.

In some embodiments, the DNA polymerase of SEQ ID NO: 2, the engineered DNA polymerase described herein, or the other DNA polymerases in Table 10.2 are useful in diagnostic applications, e.g., for detecting the presence of a target nucleic acid/polynucleotide, including DNA and RNA. In some embodiments, a method for detecting presence of a target nucleic acid/polynucleotide comprises reacting a sample suspected of containing a target nucleic acid/polynucleotide with a DNA polymerase comprising an amino acid sequence comprising residues 12 to 604 of SEQ ID NO: 2, or an amino acid sequence comprising SEQ ID NO: 2; an engineered DNA polymerase described herein; or a DNA polymerase presented in Table 10.2, in presence of substrates under conditions suitable for DNA polymerase-mediated production of a DNA complementary to all or a portion (i.e., whole or in part) of the target nucleic acid/polynucleotide, and detecting the presence of the complementary DNA. In some embodiments, the target nucleic acid/polynucleotide is DNA. In some embodiments, the target nucleic acid/polynucleotide is RNA. In some embodiments, the target nucleic acid/polynucleotide comprises a mixture of DNA and RNA.

In some embodiments, the suitable reaction conditions, particularly for the engineered DNA polymerases, comprises a reaction temperature of 15 to 80 °C, 15 to 75 °C, 20 to 70 °C, 25 to 65 °C, 30 to 60 °C, or 35 to 55 °C. In some embodiments, the reaction temperature is about 15 °C, 20 °C, 25 °C, 35 °C, 40 °C, 45 °C, 50 °C, 55 °C, 60 °C, 65 °C, 70 °C, 75 °C or 80 °C.

In some embodiments, the sample can be any material or substance suspected of containing a target nucleic acid. In some embodiments, the sample is a biological sample, such as biopsy and autopsy samples, frozen sections taken for histological purposes, blood, plasma, serum, sputum, stool, tears, mucus, hair, skin, etc. In some embodiments, the biological sample are cells or viruses, such as from a bacterial culture, virus culture, or cell culture. In some embodiments, the sample is an environmental sample, including, among others, water, including samples from ocean, river, refuse/sewer, etc., soil, air, vents, or surfaces, such as floors, machinery, counters, etc.

In some embodiments, the detection of the complementary DNA product produced by the DNA polymerase can be effectuated by methods known in the art. In some embodiments, the complementary DNA is detected by amplifying the complementary DNA, such as by PCR or isothermal amplification. In some embodiments, isothermal amplification methods for use with the engineered DNA polymerases include, among others, LAMP, whole genome amplification (WGA), and multiple displacement amplification. In some embodiments, one or more of the nucleotide substrates can be labeled, and depending on the label, detected directly (e.g., fluorescently labeled nucleotide) or indirectly (e.g., biotin labeled nucleotides). In some embodiments, one or more of a primer used in the DNA polymerase reaction can be labeled.

In some embodiments, the engineered DNA polymerase is used to detect a target RNA, where the reverse transcriptase activity of the engineered DNA polymerase described herein is used to prepare a DNA copy of the target RNA. In some embodiments, the DNA polymerase is also used to amplify the DNA copy of the target RNA. In some embodiments, the reverse transcriptase reaction is conducted separately from an amplification reaction with the DNA polymerase. In some embodiments, the reverse transcriptase and the DNA amplification reactions with the DNA polymerase are conducted sequentially. In some embodiments, the reverse transcriptase and the DNA amplification reactions with the DNA polymerase are conducted simultaneously or concurrently. In some embodiments, the reverse transcriptase reaction and DNA amplification reaction is a “one pot” reaction. In some embodiments, the reverse transcriptase reaction and DNA amplification reaction is conducted in separate reactions.

In some embodiments, a method of amplifying a target DNA or RNA, comprises contacting a target DNA or RNA with a DNA polymerase comprising an amino acid sequence comprising residues 12 to 604 of SEQ ID NO: 2, or an amino acid sequence comprising SEQ ID NO: 2; an engineered DNA polymerase described herein; or a DNA polymerase provided in Table 10.2 in presence of substrates under conditions suitable for amplifying the target DNA or RNA. In some embodiments, amplifying the DNA or RNA is by isothermal amplification, such as by LAMP.

In some embodiments, the engineered DNA polymerase is used for sequencing nucleic acids. Various methods for sequencing DNA, particularly NGS sequencing methods, are well known in the art. In some embodiments, a method of sequencing a target DNA comprises contacting a target DNA with a DNA polymerase comprising an amino acid sequence comprising residues 12 to 604 of SEQ ID NO: 2, or an amino acid sequence comprising SEQ ID NO: 2; or an engineered DNA polymerase described herein; or a DNA polymerase provided in Table 10.2 in presence of substrates appropriate for sequencing under conditions suitable for DNA polymerase mediated extension of a complementary DNA of the target DNA, and determining the sequence of the target DNA.

In a further aspect, the present disclosure provides a kit comprising a DNA polymerase having an amino acid sequence comprising residues 12 to 604 of SEQ ID NO: 2, or an amino acid sequence comprising SEQ ID NO: 2; or at least one engineered DNA polymerase disclosed herein; or at least one DNA polymerase provided in Table 10.2, as described herein. In some embodiments, the kit further comprises one or more of a buffer, a nucleotide substrate, and/or an oligonucleotide primer. In some embodiments, the kit can include multiple (e.g., two or more) oligonucleotide primers, for example to different portions of a target nucleic acid. In some embodiments, the kit further comprises a template DNA or RNA, for example a control template DNA or RNA of defined sequence and/or amount to use as a positive control for detection of a target DNA or RNA. In some embodiments, the kit comprises a second DNA polymerase, such as Taq or Pfu DNA polymerase.

### EXAMPLES

The following Examples, including experiments and results achieved, are provided for illustrative purposes only and are not to be construed as limiting the present invention.

In the experimental disclosure below, the following abbreviations where relevant apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); 1d and μL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); rcf (relative centrifugal force); psi and PSI (pounds per square inch); ° C (degrees Centigrade); RT and rt (room temperature); NGS (next-generation sequencing); ds (double stranded); ss (single stranded); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); ddH2O (double distilled water); PBS (phosphate buffered saline); BSA (bovine serum albumin); DTT (dithiothreitol); CAM (chloramphenicol); CAT (chloramphenicol acetyltransferase); IPTG (isopropyl β-D-1-thiogalactopyranoside); FIOPC (fold improvements over positive control) or FIOP (fold improvement over parent); LB (Luria-Bertani); TB (Terrific-Broth); SPRI (solid phase reversible immobilization); GITC (guanidine thiocyanate); CDC (Center for Disease Control, USA).

### Example 1

### E. coli Expression Hosts Containing Recombinant DNA Polymerase Genes

The initial DNA polymerase enzyme used to produce the variants of the present disclosure was SEQ ID NO: 2, which is the large fragment (i.e., amino acid residues 285 to 876) of the wild-type DNA polymerase of Parageobacillus genomosp. 1, cloned into the expression vector pCK110900 (See, FIG. 3 of US Pat. Appln. Publn. No. 2006/0195947) operably linked to the lac promoter under control of the lacl repressor. The expression vector also contains the P15a origin of replication and the chloramphenicol resistance gene. E. coli W3110 were transformed with the resulting plasmids, using standard methods known in the art. The transformants were isolated by subjecting the cells to chloramphenicol selection, as known in the art (see e.g., U.S. Pat. No. 8,383,346 and WO2010/144103).

### Example 2

### Preparation of HTP Reverse Transcriptase (RT)-Containing Wet Cell Pellets

E. coli cells containing recombinant DNA polymerase-encoding genes from monoclonal colonies were inoculated into 180 μL LB containing 1% glucose and 30 μg/mL chloramphenicol (CAM) in the wells of 96-well, shallow-well microtiter plates. The plates were sealed with O₂-permeable seals, and cultures were grown overnight at 30 °C, 200 rpm, and 85% humidity. Then 10 μL of each of the cell cultures were transferred into the wells of 96-well, deep-well plates containing 390 mL TB and 30 μg/mL CAM. The deep-well plates were sealed with O₂-permeable seals and incubated at 30 °C, 250 rpm, and 85% humidity until OD₆₀₀ 0.6-0.8 was reached. The cell cultures were then induced by IPTG to a final concentration of 1 mM and incubated overnight under the same conditions as originally used. The cells were then pelleted using centrifugation at 4,000 rpm for 10 min. The supernatants were discarded, and the pellets were frozen at −80 °C prior to lysis.

### Example 3

### Preparation of HTP DNA Polymerase-Containing Cell Lysates

For preparing cell lysates, 200 μL buffer containing 50 mM Triethanolamine-HCl, pH 7.5 were added to the cell paste in each well, produced as described in Example 2. The plates were shaken on a bench-top shaker to resuspend the cells. The suspensions (100 μL) were transferred to a 96-well hard-shell PCR plate and lysed for 60 min in a thermocycler at a set lysis temperature ranging from 68 °C to 80 °C, specified in the specific example. The plate was then centrifuged for 15 min at 4,000 rpm and 4 °C. The clarified supernatant liquids were optionally diluted and used in biocatalytic reactions to determine their activity, RNA sensitivity and thermostability levels.

### Example 4

### Use of Heat-Treated Lysate for RT-LAMP Reactions for SARS-CoV-2 Detection

RT-LAMP assays were conducted using a primer set targeting CoV-Orf1a consisting of six oligonucleotides at different concentrations (final concentrations FIP: 1.6 uM, BIP: 1.6 uM, F3: 0.2 uM, B3: 0.2 uM, loop F: 0.4 uM, loop B: 0.5 uM). Each variant was screened in a 5 uL reaction that comprised SARS-CoV-2 synthetic RNA control 1 (GenBank/GISAID ID MT007544.1, Twist Biosciences part number 102019, 50-1250 copies per reaction as specified in example), 0.5 uL 10× isothermal amplification buffer (NEB catalog #B0537S), 0.5 uL 10× Orf1a LAMP primer mixture (FIP: 16 uM, BIP: 16 uM, F3: 2 uM, B3: 2 uM, loop F: 4 uM, loop B: 4 uM), dNTP mix (final concentration 1.1 mM each dNTP), 0.3 uL 100 mM MgSO₄ (final concentration 6 mM; 8 mM total including contribution from 10× isothermal amplification buffer), 0.25 uL EvaGreen® (20× in water stock, 1.25 uM final concentration), 2 uL heat-treated E. coli lysate harboring DNA polymerase variants (lysate is diluted prior to addition to reaction as specified in following examples). Synthetic RNA control was added last to initiate the reaction. The 384-well PCR plate was briefly vortexed and centrifuged to mix, then inserted into a CFX Touch 384-well Real-Time PCR Detection System (Bio-Rad). The isothermal amplification reaction was performed at 65 °C for 120 cycles of 30 seconds, with the reactions' fluorescence analyzed in the FAM channel after every cycle.

| | SEQ | |
|---|---|---|
| Primer | ID | |
| name | NO | Sequence |
| Orfla-F3 | 533 | CGGTGGACAAATTGTCAC |
| Orfla-B3 | 534 | CTTCTCTGGATTTAACACACTT |
| Orfla-LF | 535 | TTACAAGCTTAAAGAATGTCTGAACACT |
| Orfla-LB | 536 | TTGAATTTAGGTGAAACATTTGTCACG |
| Orfla-FIP | 537 | TCAGCACACAAAGCCAAAAATTTATCT |
| | | GTGCAAAGGAAATTAAGGAG |
| Orfla-BIP | 538 | TATTGGTGGAGCTAAACTTAAAGCC |
| | | CTGTACAATCCCTTTGAGTG |

### Example 5

### Improvements Over SEQ ID NO: 2 in Thermostability

The large fragment of the DNA polymerase of SEQ ID NO: 2 described in Example 1 was selected as the parent enzyme after screening the large fragment of various wild-type enzymes (see Table 10.2) for both reverse transcriptase and LAMP activity in an RT-LAMP reaction in the absence of a separate reverse transcriptase enzyme. Libraries of engineered genes were produced using established techniques (e.g., saturation mutagenesis, recombination of previously identified beneficial mutations). The polypeptides encoded by each gene were produced in HTP as described in Example 2, and the soluble lysate was generated as described in Example 3, with a lysis temperature of 68 °C. The clarified lysate was diluted four-fold prior to setting up the RT-LAMP reactions with 1250 copies SARS-CoV-2 RNA as described in Example 4.

Residual activity after heat treatment relative to SEQ ID NO: 2 (activity FIOP) was calculated as the inverse of the quantification cycle (Cq) value and is shown in Table 5.1. Samples which do not have detectable signal above the threshold value after 120 cycles (including positive and negative controls with no detectable signal) are set to a Cq of 120 (the maximum cycles measured) rather than not detected (n.d.) to enable fold-improvement calculations.

TABLE 5.1
Activity of Variants Relative to SEQ ID NO: 2

| | | Activity |
|---|---|---|
| | | FIOP |
| SEQ | | (Relative |
| ID NO: | Amino Acid Differences | to SEQ |
| (nt/aa) | (Relative to SEQ ID NO: 2) | ID NO: 2) |
| 3/4 | F509R | +++ |
| 5/6 | E300R | +++ |
| 7/8 | N452R | +++ |
| 9/10 | S36T/A241Q/K372E/I470S | +++ |
| 11/12 | I124L/D192E/S210A/K372E/P427A/P456T/M552L | +++ |
| 13/14 | I124L | +++ |
| 15/16 | E52G | ++ |
| 17/18 | Q483K | ++ |
| 19/20 | K372E | ++ |
| 21/22 | N452S | ++ |
| 23/24 | E300A | ++ |
| 25/26 | I393L | ++ |
| 27/28 | E300G | ++ |
| 29/30 | I212V | ++ |
| 31/32 | E52G/A66T | ++ |
| 33/34 | Q133R | ++ |
| 35/36 | N454G | + |
| 37/38 | E154W | + |
| 39/40 | D593S | + |
| 41/42 | E462R | + |
| 43/44 | D541R | + |
| 45/46 | E21M | + |
| 47/48 | Q573V | + |
| 49/50 | T505H | + |
| 51/52 | S152R | + |
| 53/54 | P294R | + |
| 55/56 | R545K | + |
| 57/58 | G101A/A241Q/I470S | + |
| 59/60 | I584V | + |
| 61/62 | H304L | + |
| 63/64 | Q295K | + |
| 65/66 | E578F | + |
| 67/68 | D541T | + |
| 69/70 | P456T/I470S | + |
| 71/72 | E253R | + |
| 73/74 | E290G | + |
| 75/76 | D192E/A241E/K372E/P456T | + |
| 77/78 | Q252T | + |

¹ Levels of increased activity were determined relative to the reference polypeptide of SEQ ID NO: 2 and defined as follows: “+” 1.29 to 2.50-fold increased activity; “++” > 2.50-fold increased activity; “+++” > 3.50-fold increased activity

### Example 6

### Improvements Over SEQ ID NO: 10 in Thermostability

SEQ ID NO: 10 was selected as the parent enzyme for this round of directed evolution. Libraries of engineered genes were produced using established techniques (e.g., saturation mutagenesis, recombination of previously identified beneficial mutations). The polypeptides encoded by each gene were produced in HTP as described in Example 2, and the soluble lysate was generated as described in Example 3 with replicate plates lysed with lysis temperatures of 71 °C, 72 °C, 73.5 °C, and 75 C. The clarified lysate was diluted eight-fold prior to setting up the RT-LAMP reactions with 1250 copies SARS-CoV-2 RNA as described in Example 4.

Residual activity after heat treatment relative to SEQ ID NO: 10 (activity FIOP) was calculated as the inverse of the quantification cycle (Cq) value and is shown in Table 6.1. Samples which do not have detectable signal above the threshold value after 120 cycles (including positive and negative controls with no detectable signal) are set to a Cq of 120 (the maximum cycles measured) rather than not detected (n.d.) to enable fold-improvement calculations.

TABLE 6.1
Activity and Stability of Variants Relative to SEQ ID NO: 10

| | | | Activity | Activity | Activity |
|---|---|---|---|---|---|
| | | | FIOP after | FIOP after | FIOP after |
| | | | 71 °C heat | 72 °C heat | 73.5 °C heat |
| | Amino Acid | Amino Acid | treatment ¹ | treatment ¹ | treatment ¹ |
| SEQ ID | Differences | Differences | (Relative to | (Relative to | (Relative to |
| NO: | (Relative to | (Relative to | SEQ ID | SEQ ID | SEQ ID |
| (nt/aa) | SEQ ID NO: 10) | SEQ ID NO: 2) | NO: 10) | NO: 10 | NO: 10) |
| 79/80 | E52G/G101A/I124L/ | S36T/E52G/G101A/I124L/ | +++ | +++ | +++ |
| | I212V/P294R/E372K/ | I212V/A241Q/P294R/ | | | |
| | I393L/N452R/ | I393L/N452R/I470S/ | | | |
| | Q483K/F509R | Q483K/F509R | | | |
| 81/82 | E52G/I124L/E300A/ | S36T/E52G/I124L/A241Q/ | +++ | ++ | +++ |
| | I393L/N452R | E300A/K372E/I393L/ | | | |
| | | N452R/I470S | | | |
| 83/84 | E154W/I212V/P294R/ | S36T/E154W/I212V/A241Q/ | ++ | +++ | +++ |
| | E300G/E372K/ | P294R/E300G/I393L/ | | | |
| | I393L/N452R/Q483K/ | N452R/I470S/Q483K/ | | | |
| | F509R | F509R | | | |
| 85/86 | E52G/G101A/E154W/ | S36T/E52G/G101A/E154W/ | +++ | +++ | +++ |
| | P294R/E300R/ | A241Q/P294R/E300R/ | | | |
| | N452S/F509R/D593S | K372E/N452S/I470S/ | | | |
| | | F509R/D593S | | | |
| 87/88 | E52G/E154W/I212V/ | S36T/E52G/E154W/I212V/ | ++ | ++ | +++ |
| | P294R/E300G/I393L/ | A241Q/P294R/E300G/ | | | |
| | N452R | K372E/I393L/N452R/I470S | | | |
| 89/90 | I212V/E300R/I393L/ | S36T/I212V/A241Q/E300R/ | +++ | +++ | +++ |
| | N452R/F509R | K372E/I393L/N452R/ | | | |
| | | I470S/F509R | | | |
| 91/92 | E52G/G101A/I212V/ | S36T/E52G/G101A/I212V/ | +++ | +++ | ++ |
| | P294R/E300R/ | A241Q/P294R/E300R/ | | | |
| | I393L/N452R/Q483K/ | K372E/I393L/N452R/ | | | |
| | F509R/D593S | I470S/Q483K/F509R/ | | | |
| | | D593S | | | |
| 93/94 | E52G/I124L/P294R/ | S36T/E52G/I124L/A241Q/ | +++ | +++ | ++ |
| | E300R/N452S/ | P294R/E300R/K372E/ | | | |
| | F509R | N452S/I470S/F509R | | | |
| 95/96 | I124L/E300R/I393L/ | S36T/I124L/A241Q/E300R/ | +++ | +++ | ++ |
| | N452R/Q483K/ | K372E/I393L/N452R/ | | | |
| | F509R/D593S | I470S/Q483K/F509R/ | | | |
| | | D593S | | | |
| 97/98 | G101A/I124L/I212V/ | S36T/G101A/I124L/I212V/ | +++ | +++ | ++ |
| | N452R/Q483K/ | A241Q/K372E/N452R/ | | | |
| | F509R | I470S/Q483K/F509R | | | |
| 99/100 | E52G/I393L/N452R/ | S36T/E52G/A241Q/K372E/ | +++ | +++ | ++ |
| | F509R/D593S | I393L/N452R/I470S/ | | | |
| | | F509R/D593S | | | |
| 101/102 | I212V/E300R/N452R/ | S36T/I212V/A241Q/E300R/ | ++ | ++ | ++ |
| | F509R/D593S | K372E/N452R/I470S/ | | | |
| | | F509R/D593S | | | |
| 103/104 | E52G/N452R/ | S36T/E52G/A241Q/K372E/ | ++ | ++ | ++ |
| | F509R/D593S | N452R/I470S/F509R/ | | | |
| | | D593S | | | |
| 105/106 | E154W/I212V/E300R/ | S36T/E154W/I212V/ | ++ | ++ | ++ |
| | E372K/N452R/ | A241Q/E300R/N452R/ | | | |
| | F509R/D593S | I470S/F509R/D593S | | | |
| 107/108 | E52G/I124L/I212V/ | S36T/E52G/I124L/I212V/ | ++ | ++ | ++ |
| | P294R/I393L/ | A241Q/P294R/K372E/ | | | |
| | N452R/D593S | I393L/N452R/I470S/D593S | | | |
| 109/110 | I124L/N452R/F509R | S36T/I124L/A241Q/K372E/ | ++ | ++ | ++ |
| | | N452R/I470S/F509R | | | |
| 111/112 | S152R/E253R/D287R/ | S36T/S152R/A241Q/E253R/ | +++ | +++ | ++ |
| | T505H/D541R/ | D287R/K372E/I470S/ | | | |
| | Q573V/I584V | T505H/D541R/Q573V/ | | | |
| | | I584V | | | |
| 113/114 | E154W/I212V/ | S36T/E154W/I212V/ | ++ | ++ | ++ |
| | E300R/N452R | A241Q/E300R/K372E/ | | | |
| | | N452R/I470S | | | |
| 115/116 | S152R/E253R/D287R/ | S36T/S152R/A241Q/E253R/ | ++ | ++ | ++ |
| | H304L/N454G/ | D287R/H304L/K372E/ | | | |
| | R545K/Q573V/I584V | N454G/I470S/R545K/ | | | |
| | | Q573V/I584V | | | |
| 117/118 | E52G/I124L/E154W/ | S36T/E52G/I124L/E154W/ | ++ | ++ | ++ |
| | P294R/E300A/ | A241Q/P294R/E300A/ | | | |
| | E372K/N452R/F509R | N452R/I470S/F509R | | | |
| 119/120 | P294R/E300R/E372K/ | S36T/A241Q/P294R/E300R/ | +++ | ++ | ++ |
| | N452S/F509R/ | N452S/I470S/F509R/ | | | |
| | D593S | D593S | | | |
| 121/122 | E52G/I212V/N452R | S36T/E52G/I212V/A241Q/ | +++ | +++ | + |
| | | K372E/N452R/I470S | | | |
| 123/124 | I124L/P294R/ | S36T/I124L/A241Q/P294R/ | ++ | ++ | + |
| | I393L/N452R | K372E/I393L/N452R/ | | | |
| | | I470S | | | |
| 125/126 | E52G/E300R/ | S36T/E52G/A241Q/E300R/ | +++ | +++ | + |
| | F509R/D593S | K372E/I470S/F509R/ | | | |
| | | D593S | | | |
| 127/128 | S152R/E253R/D287R/ | S36T/S152R/A241Q/E253R/ | +++ | +++ | + |
| | N454G/D541R/ | D287R/K372E/N454G/ | | | |
| | I584V | I470S/D541R/I584V | | | |
| 129/130 | E52G/G101A/N452S/ | S36T/E52G/G101A/A241Q/ | +++ | +++ | + |
| | F509R | K372E/N452S/I470S/ | | | |
| | | F509R | | | |
| 131/132 | E52G/E154W/I212V/ | S36T/E52G/E154W/I212V/ | +++ | +++ | + |
| | E300R/E372K/I393L/ | A241Q/E300R/I393L/ | | | |
| | F509R | I470S/F509R | | | |
| 133/134 | E21M/E253R/D287R/ | E21M/S36T/A241Q/E253R/ | +++ | +++ | + |
| | N454G/I584V | D287R/K372E/N454G/ | | | |
| | | I470S/I584V | | | |
| 135/136 | E52G/I393L/N452R | S36T/E52G/A241Q/K372E/ | +++ | ++ | + |
| | | I393L/N452R/I470S | | | |
| 137/138 | E52G/I124L/E154W/ | S36T/E52G/I124L/E154W/ | +++ | ++ | + |
| | P294R/F509R | A241Q/P294R/K372E/ | | | |
| | | I470S/F509R | | | |
| 139/140 | G101A/I124L/I212V/ | S36T/G101A/I124L/I212V/ | ++ | +++ | + |
| | E300R/I393L | A241Q/E300R/K372E/ | | | |
| | | I393L/I470S | | | |
| 141/142 | I124L/P294R/E300G/ | S36T/I124L/A241Q/P294R/ | ++ | +++ | + |
| | E372K/N452S/F509R/ | E300G/N452S/I470S/ | | | |
| | D593S | F509R/D593S | | | |
| 143/144 | E52G/I124L/E154W/ | S36T/E52G/I124L/E154W/ | ++ | ++ | + |
| | P294R/Q483K/ | A241Q/P294R/K372E/ | | | |
| | D593S | I470S/Q483K/D593S | | | |
| 145/146 | E52G/E372K/I393L/ | S36T/E52G/A241Q/I393L/ | +++ | +++ | + |
| | N452S/F509R/D593S | N452S/I470S/F509R/ | | | |
| | | D593S | | | |
| 147/148 | E154W/E300R/I393L/ | S36T/E154W/A241Q/E300R/ | ++ | +++ | + |
| | N452S/D593S | K372E/I393L/N452S/ | | | |
| | | I470S/D593S | | | |
| 149/150 | E52G/G101A/P294R/ | S36T/E52G/G101A/A241Q/ | ++ | + | + |
| | E372K/I393L/N452S/ | P294R/I393L/N452S/ | | | |
| | F509R | I470S/F509R | | | |
| 151/152 | E21M/S152R/E253R/ | E21M/S36T/S152R/A241Q/ | ++ | ++ | |
| | D287R/H304L/ | E253R/D287R/H304L/ | | | |
| | N454G/T505H | K372E/N454G/I470S/ | | | |
| | | T505H | | | |
| 153/154 | E52G/E300R/I393L | S36T/E52G/A241Q/E300R/ | ++ | ++ | |
| | | K372E/I393L/I470S | | | |
| 155/156 | G101A/I124L/E154W/ | S36T/G101A/I124L/E154W/ | +++ | ++ | |
| | P294R/N452S/ | A241Q/P294R/K372E/ | | | |
| | F509R | N452S/I470S/F509R | | | |
| 157/158 | E154W/I212V/E372K/ | S36T/E154W/I212V/A241Q/ | +++ | ++ | |
| | I393L/F509R/ | I393L/I470S/F509R/ | | | |
| | D593S | D593S | | | |
| 159/160 | E21M/E253R/D287R/ | E21M/S36T/A241Q/E253R/ | ++ | ++ | |
| | N454G/T505H/ | D287R/K372E/N454G/ | | | |
| | D541G | I470S/T505H/D541G | | | |
| 161/162 | D287R/N454G/T505H/ | S36T/A241Q/D287R/K372E/ | +++ | ++ | |
| | I584V | N454G/I470S/T505H/ | | | |
| | | I584V | | | |
| 163/164 | E52G/I124L/P294R/ | S36T/E52G/I124L/A241Q/ | ++ | ++ | |
| | E300R/E372K/N452S | P294R/E300R/N452S/I470S | | | |
| 165/166 | D287R/T505H/D541R/ | S36T/A241Q/D287R/K372E/ | ++ | ++ | |
| | Q573V | I470S/T505H/D541R/ | | | |
| | | Q573V | | | |
| 167/168 | E253R/T505H/I584V | S36T/A241Q/E253R/K372E/ | + | ++ | |
| | | I470S/T505H/I584V | | | |
| 169/170 | L84Q/E253R/D287R/ | S36T/L84Q/A241Q/E253R/ | + | ++ | |
| | P456T/Q573V | D287R/K372E/P456T/ | | | |
| | | I470S/Q573V | | | |
| 171/172 | E52G/E372K/F509R/ | S36T/E52G/A241Q/I470S/ | ++ | ++ | |
| | D593S | F509R/D593S | | | |
| 173/174 | F509R/D593S | S36T/A241Q/K372E/I470S/ | ++ | ++ | |
| | | F509R/D593S | | | |
| 175/176 | E154W/I212V/E300R/ | S36T/E154W/I212V/A241Q/ | ++ | ++ | |
| | E372K/F509R | E300R/I470S/F509R | | | |
| 177/178 | E52G/E154W/P294R/ | S36T/E52G/E154W/A241Q/ | ++ | ++ | |
| | F509R/D593S | P294R/K372E/I470S/ | | | |
| | | F509R/D593S | | | |
| 179/180 | S152R/Q295K/T505H/ | S36T/S152R/A241Q/Q295K/ | ++ | ++ | |
| | R545K/Q573V | K372E/I470S/T505H/ | | | |
| | | R545K/Q573V | | | |
| 181/182 | E253R/D541R/I584V | S36T/A241Q/E253R/K372E/ | + | ++ | |
| | | I470S/D541R/I584V | | | |
| 183/184 | E253R/Q295K/H304L/ | S36T/A241Q/E253R/Q295K/ | ++ | ++ | |
| | N454G/E462R/ | H304L/K372E/N454G/ | | | |
| | Q573V/I584V | E462R/I470S/Q573V/ | | | |
| | | I584V | | | |
| 185/186 | E253R/N454G/T505H/ | S36T/A241Q/E253R/K372E/ | + | ++ | |
| | Q573V/I584V | N454G/I470S/T505H/ | | | |
| | | Q573V/I584V | | | |
| 187/188 | E154W/I212V/E300R | S36T/E154W/I212V/A241Q/ | ++ | ++ | |
| | | E300R/K372E/I470S | | | |
| 189/190 | D287R/N454G/R545K/ | S36T/A241Q/D287R/K372E/ | + | + | |
| | Q573V | N454G/I470S/R545K/ | | | |
| | | Q573V | | | |
| 191/192 | E21M/S152R/D287R/ | E21M/S36T/S152R/A241Q/ | + | + | |
| | H304L/N454G | D287R/H304L/K372E/ | | | |
| | | N454G/I470S | | | |
| 193/194 | S152R/E253R/Q295K/ | S36T/S152R/A241Q/E253R/ | ++ | + | |
| | H304L/D541R/ | Q295K/H304L/K372E/ | | | |
| | Q573V | I470S/D541R/Q573V | | | |
| 195/196 | S152R/E253R/D287R/ | S36T/S152R/A241Q/E253R/ | ++ | + | |
| | H304L/N454G | D287R/H304L/K372E/ | | | |
| | | N454G/I470S | | | |
| 197/198 | S152R/N454G/Q573V | S36T/S152R/A241Q/K372E/ | + | + | |
| | | N454G/I470S/Q573V | | | |
| 199/200 | I124L/P294R/E372K/ | S36T/I124L/A241Q/P294R/ | +++ | + | |
| | F509R/D593S | I470S/F509R/D593S | | | |
| 201/202 | S152R/E253R/Q295K/ | S36T/S152R/A241Q/E253R/ | + | + | |
| | N454G | Q295K/K372E/N454G/ | | | |
| | | I470S | | | |
| 203/204 | E154W/E300R/N452S | S36T/E154W/A241Q/E300R/ | + | + | |
| | | K372E/N452S/I470S | | | |
| 205/206 | D287R/H304L/T505H | S36T/A241Q/D287R/H304L/ | + | + | |
| | | K372E/I470S/T505H | | | |
| 207/208 | S152R/E253R/N454G/ | S36T/S152R/A241Q/E253R/ | + | + | |
| | T505H | K372E/N454G/I470S/ | | | |
| | | T505H | | | |
| 209/210 | E253R/N454G/T505H | S36T/A241Q/E253R/K372E/ | + | + | |
| | | N454G/I470S/T505H | | | |
| 211/212 | E21M/E253R/N454G/ | E21M/S36T/A241Q/E253R/ | ++ | + | |
| | T505H/R545K | K372E/N454G/I470S/ | | | |
| | | T505H/R545K | | | |
| 213/214 | S152R/E253R/D287R/ | S36T/S152R/A241Q/E253R/ | + | + | |
| | T505H | D287R/K372E/I470S/ | | | |
| | | T505H | | | |
| 215/216 | T505H/D541R/I584V | S36T/A241Q/K372E/I470S/ | + | + | |
| | | T505H/D541R/I584V | | | |
| 217/218 | S152R/E253R/N454G/ | S36T/S152R/A241Q/E253R/ | + | + | |
| | I584V | K372E/N454G/I470S/ | | | |
| | | I584V | | | |

¹ Levels of increased activity following temperature challenges at 71 °C, 72 °C, and 73.5 °C were determined relative to the reference polypeptide of SEQ ID NO: 10 and defined as follows: “+” 1.02 to 2.50, “++” > 2.50, “+++” > 4.50

### Example 7

### Improvements Over SEQ ID NO: 80 in Thermostability

SEQ ID NO: 80 was selected as the parent enzyme for this round of directed evolution. Libraries of engineered genes were produced using well established techniques (e.g., saturation mutagenesis, recombination of previously identified beneficial mutations). The polypeptides encoded by each gene were produced in HTP as described in Example 2, and the soluble lysate was generated as described in Example 3 with replicate plates lysed with lysis temperatures of 77 °C, 78 °C, and 79 °C. The clarified lysate was diluted eight-fold prior to setting up the RT-LAMP reactions with 1000 copies SARS-CoV-2 RNA as described in Example 4.

Residual activity after heat treatment relative to SEQ ID NO: 80 (activity FIOP) was calculated as the inverse of the quantification cycle (Cq) value and is shown in Table 7.1. Samples which do not have detectable signal above the threshold value after 120 cycles (including positive and negative controls with no detectable signal) are set to a Cq of 120 (the maximum cycles measured) rather than not detected (n.d.) to enable fold-improvement calculations.

TABLE 7.1
Activity and Stability of Variants Relative to SEQ ID NO: 80

| | | | Activity | Activity | Activity |
|---|---|---|---|---|---|
| | | | FIOP after | FIOP after | FIOP after |
| | | | 77 C heat | 78 C heat | 79 C heat |
| | Amino Acid | Amino Acid | treatment ¹ | treatment ¹ | treatment ¹ |
| SEQ ID | Differences | Differences | (Relative to | (Relative to | (Relative to |
| NO: | (Relative to | (Relative to | SEQ ID | SEQ ID | SEQ ID |
| (nt/aa) | SEQ ID NO: 80) | SEQ ID NO: 2) | NO: 80) | NO: 80) | NO: 80) |
| 219/220 | E300R/N454G/ | S36T/E52G/G101A/I124L/ | ++ | +++ | +++ |
| | P456T/D541R/ | I212V/A241Q/P294R/E300R/ | | | |
| | I584V | I393L/N452R/N454G/ | | | |
| | | P456T/I470S/Q483K/F509R/ | | | |
| | | D541R/I584V | | | |
| 221/222 | E21M/E300R/ | E21M/S36T/E52G/G101A/ | +++ | +++ | +++ |
| | N454G/R545K/ | I124L/I212V/A241Q/P294R/ | | | |
| | I584V | E300R/I393L/N452R/ | | | |
| | | N454G/I470S/Q483K/ | | | |
| | | F509R/R545K/I584V | | | |
| 223/224 | E253R/E300R/ | S36T/E52G/G101A/I124L/ | +++ | +++ | +++ |
| | N454G/P456T/ | I212V/A241Q/E253R/P294R/ | | | |
| | I584V | E300R/I393L/N452R/ | | | |
| | | N454G/P456T/I470S/Q483K/ | | | |
| | | F509R/I584V | | | |
| 225/226 | E154W/E253R/ | S36T/E52G/G101A/I124L/ | +++ | +++ | +++ |
| | E300R/N454G/ | E154W/I212V/A241Q/ | | | |
| | P456T/T505H/ | E253R/P294R/E300R/I393L/ | | | |
| | Q573V | N452R/N454G/P456T/ | | | |
| | | 470S/Q483K/T505H/F509R/ | | | |
| | | Q573V | | | |
| 227/228 | E21M/E154W/ | E21M/S36T/E52G/G101A/ | +++ | ++ | +++ |
| | E300R/N454G/ | I124L/E154W/I212V/A241Q/ | | | |
| | P456T/Q573V | P294R/E300R/I393L/ | | | |
| | | N452R/N454G/P456T/I470S/ | | | |
| | | Q483K/F509R/Q573V | | | |
| 229/230 | S152R/E300R/ | S36T/E52G/G101A/I124L/ | +++ | ++ | +++ |
| | N454G/P456T/ | S152R/I212V/A241Q/P294R/ | | | |
| | T505H/I584V/ | E300R/I393L/N452R/ | | | |
| | D593S | N454G/P456T/I470S/Q483K/ | | | |
| | | T505H/F509R/I584V/D593S | | | |
| 231/232 | E154W/E253R/ | S36T/E52G/G101A/I124L/ | +++ | +++ | +++ |
| | E300R/P456T/ | E154W/I212V/A241Q/ | | | |
| | T505H/R545K/ | E253R/P294R/E300R/I393L/ | | | |
| | Q573V/I584V | N452R/P456T/I470S/ | | | |
| | | Q483K/T505H/F509R/R545K/ | | | |
| | | Q573V/I584V | | | |
| 233/234 | E253R/E300R/ | S36T/E52G/G101A/I124L/ | +++ | ++ | +++ |
| | N454G/P456T/ | I212V/A241Q/E253R/P294R/ | | | |
| | T505H/Q573V | E300R/I393L/N452R/ | | | |
| | | N454G/P456T/I470S/Q483K/ | | | |
| | | T505H/F509R/Q573V | | | |
| 235/236 | E154W/E253R/ | S36T/E52G/G101A/I124L/ | +++ | ++ | ++ |
| | E300R/N454G/ | E154W/I212V/A241Q/ | | | |
| | P456T/D541G/ | E253R/P294R/E300R/I393L/ | | | |
| | Q573V/I584V | N452R/N454G/P456T/ | | | |
| | | I470S/Q483K/F509R/D541G/ | | | |
| | | Q573V/I584V | | | |
| 237/238 | E154W/E300R/ | S36T/E52G/G101A/I124L/ | +++ | ++ | ++ |
| | N454G/T505H | E154W/I212V/A241Q/ | | | |
| | | P294R/E300R/I393L/N452R/ | | | |
| | | N454G/I470S/Q483K/ | | | |
| | | T505H/F509R | | | |
| 239/240 | E21M/E253R/ | E21M/S36T/E52G/G101A/ | +++ | ++ | ++ |
| | E300R/N454G/ | I124L/I212V/A241Q/E253R/ | | | |
| | P456T/R545K/ | P294R/E300R/I393L/ | | | |
| | Q573V/D593S | N452R/N454G/P456T/I470S/ | | | |
| | | Q483K/F509R/R545K/ | | | |
| | | Q573V/D593S | | | |
| 241/242 | E168G/E300R/ | S36T/E52G/G101A/I124L/ | +++ | +++ | ++ |
| | N454G/P456T/ | E168G/I212V/A241Q/P294R/ | | | |
| | R545K/Q573V | E300R/I393L/N452R/ | | | |
| | | N454G/P456T/I470S/Q483K/ | | | |
| | | F509R/R545K/Q573V | | | |
| 243/244 | E253R/E300R/ | S36T/E52G/G101A/I124L/ | +++ | + | ++ |
| | N454G/T505H/ | I212V/A241Q/E253R/P294R/ | | | |
| | Q573V | E300R/I393L/N452R/ | | | |
| | | N454G/I470S/Q483K/ | | | |
| | | T505H/F509R/Q573V | | | |
| 245/246 | E154W/E300R/ | S36T/E52G/G101A/I124L/ | +++ | ++ | ++ |
| | T505H/R545K/ | E154W/I212V/A241Q/ | | | |
| | I584V | P294R/E300R/I393L/N452R/ | | | |
| | | I470S/Q483K/T505H/ | | | |
| | | F509R/R545K/I584V | | | |
| 247/248 | E154W/E253R/ | S36T/E52G/G101A/I124L/ | +++ | ++ | ++ |
| | E300R/P456T/ | E154W/I212V/A241Q/ | | | |
| | D541R/Q573V/ | E253R/P294R/E300R/I393L/ | | | |
| | I584V | N452R/P456T/I470S/ | | | |
| | | Q483K/F509R/D541R/ | | | |
| | | Q573V/I584V | | | |
| 249/250 | E21M/E154W/ | E21M/S36T/E52G/G101A/ | +++ | ++ | ++ |
| | E300R/N454G/ | I124L/E154W/I212V/A241Q/ | | | |
| | P456T/T505H/ | P294R/E300R/I393L/ | | | |
| | R545K | N452R/N454G/P456T/I470S/ | | | |
| | | Q483K/T505H/F509R/R545K | | | |
| 251/252 | E154W/E253R/ | S36T/E52G/G101A/I124L/ | +++ | ++ | ++ |
| | E300R/N454G/ | E154W/I212V/A241Q/ | | | |
| | T505H | E253R/P294R/E300R/I393L/ | | | |
| | | N452R/N454G/I470S/ | | | |
| | | Q483K/T505H/F509R | | | |
| 253/254 | E21M/E154W/ | E21M/S36T/E52G/G101A/ | +++ | ++ | ++ |
| | E253R/E300R/ | I124L/E154W/I212V/A241Q/ | | | |
| | N454G/Q573V | E253R/P294R/E300R/ | | | |
| | | I393L/N452R/N454G/I470S/ | | | |
| | | Q483K/F509R/Q573V | | | |
| 255/256 | E300R/N454G/ | S36T/E52G/G101A/I124L/ | ++ | + | ++ |
| | T505H/D541R/ | I212V/A241Q/P294R/E300R/ | | | |
| | I584V | I393L/N452R/N454G/ | | | |
| | | I470S/Q483K/T505H/F509R/ | | | |
| | | D541R/I584V | | | |
| 257/258 | E300R/N454G/ | S36T/E52G/G101A/I124L/ | +++ | + | ++ |
| | T505H/D541R/ | I212V/A241Q/P294R/E300R/ | | | |
| | Q573V | I393L/N452R/N454G/ | | | |
| | | I470S/Q483K/T505H/F509R/ | | | |
| | | D541R/Q573V | | | |
| 259/260 | E154W/E253R/ | S36T/E52G/G101A/I124L/ | +++ | ++ | ++ |
| | E300R/N454G | E154W/I212V/A241Q/ | | | |
| | | E253R/P294R/E300R/I393L/ | | | |
| | | N452R/N454G/I470S/ | | | |
| | | Q483K/F509R | | | |
| 261/262 | E154W/E300R/ | S36T/E52G/G101A/I124L/ | +++ | ++ | ++ |
| | N454G/Q573V | E154W/I212V/A241Q/ | | | |
| | | P294R/E300R/I393L/N452R/ | | | |
| | | N454G/I470S/Q483K/ | | | |
| | | F509R/Q573V | | | |
| 263/264 | E300R/N454G/ | S36T/E52G/G101A/I124L/ | +++ | ++ | ++ |
| | P456T/R545K/ | I212V/A241Q/P294R/E300R/ | | | |
| | Q573V | I393L/N452R/N454G/ | | | |
| | | P456T/I470S/Q483K/ | | | |
| | | F509R/R545K/Q573V | | | |
| 265/266 | E253R/E300R/ | S36T/E52G/G101A/I124L/ | +++ | + | ++ |
| | N454G/P456T | I212V/A241Q/E253R/P294R/ | | | |
| | | E300R/I393L/N452R/ | | | |
| | | N454G/P456T/I470S/Q483K/ | | | |
| | | F509R | | | |
| 267/268 | E154W/E253R/ | S36T/E52G/G101A/I124L/ | +++ | ++ | ++ |
| | E300R/N454G/ | E154W/I212V/A241Q/ | | | |
| | R545K/Q573V | E253R/P294R/E300R/I393L/ | | | |
| | | N452R/N454G/I470S/ | | | |
| | | Q483K/F509R/R545K/Q573V | | | |
| 269/270 | E21M/S152R/ | E21M/S36T/E52G/G101A/ | +++ | + | ++ |
| | E253R/E300R/ | I124L/S152R/I212V/A241Q/ | | | |
| | N454G/P456T/ | E253R/P294R/E300R/ | | | |
| | D541R/Q573V | I393L/N452R/N454G/P456T/ | | | |
| | | I470S/Q483K/F509R/ | | | |
| | | D541R/Q573V | | | |
| 271/272 | E154W/E253R/ | S36T/E52G/G101A/I124L/ | +++ | + | ++ |
| | E300R/N454G/ | E154W/I212V/A241Q/ | | | |
| | P456T/Q573V | E253R/P294R/E300R/I393L/ | | | |
| | | N452R/N454G/P456T/ | | | |
| | | I470S/Q483K/F509R/Q573V | | | |
| 273/274 | S152R/E300R/ | S36T/E52G/G101A/I124L/ | +++ | + | ++ |
| | N454G | S152R/I212V/A241Q/P294R/ | | | |
| | | E300R/I393L/N452R/ | | | |
| | | N454G/I470S/Q483K/F509R | | | |
| 275/276 | E154W/E253R/ | S36T/E52G/G101A/I124L/ | +++ | ++ | ++ |
| | E300R/N454G/ | E154W/I212V/A241Q/ | | | |
| | T505H/D541R/ | E253R/P294R/E300R/I393L/ | | | |
| | D593S | N452R/N454G/I470S/ | | | |
| | | Q483K/T505H/F509R/D541R/ | | | |
| | | D593S | | | |
| 277/278 | E21M/E154W/ | E21M/S36T/E52G/G101A/ | ++ | + | ++ |
| | E300R/N454G/ | I124L/E154W/I212V/A241Q/ | | | |
| | Q573V/I584V | P294R/E300R/I393L/ | | | |
| | | N452R/N454G/I470S/Q483K/ | | | |
| | | F509R/Q573V/I584V | | | |
| 279/280 | E154W/E300R/ | S36T/E52G/G101A/I124L/ | ++ | ++ | ++ |
| | P456T/Q573V/ | E154W/I212V/A241Q/ | | | |
| | D593S | P294R/E300R/I393L/N452R/ | | | |
| | | P456T/I470S/Q483K/ | | | |
| | | F509R/Q573V/D593S | | | |
| 281/282 | S152R/E253R/ | S36T/E52G/G101A/I124L/ | ++ | ++ | ++ |
| | E300R/N454G/ | S152R/I212V/A241Q/E253R/ | | | |
| | R545K/Q573V | P294R/E300R/I393L/ | | | |
| | | N452R/N454G/I470S/Q483K/ | | | |
| | | F509R/R545K/Q573V | | | |
| 283/284 | E21M/E300R/ | E21M/S36T/E52G/G101A/ | + | ++ | ++ |
| | P456T/L495F/ | I124L/I212V/A241Q/P294R/ | | | |
| | T505H/D541R/ | E300R/I393L/N452R/ | | | |
| | I584V | P456T/I470S/Q483K/L495F/ | | | |
| | | T505H/F509R/D541R/I584V | | | |
| 285/286 | E154W/E300R/ | S36T/E52G/G101A/I124L/ | ++ | + | ++ |
| | N454G/D541G/ | E154W/I212V/A241Q/ | | | |
| | I584V | P294R/E300R/I393L/N452R/ | | | |
| | | N454G/I470S/Q483K/ | | | |
| | | F509R/D541G/I584V | | | |
| 287/288 | E253R/E300R/ | S36T/E52G/G101A/I124L/ | +++ | ++ | ++ |
| | T505H/D541R/ | I212V/A241Q/E253R/P294R/ | | | |
| | Q573V/I584V | E300R/I393L/N452R/ | | | |
| | | I470S/Q483K/T505H/F509R/ | | | |
| | | D541R/Q573V/I584V | | | |
| 289/290 | E253R/E300R/ | S36T/E52G/G101A/I124L/ | +++ | + | + |
| | I584V | I212V/A241Q/E253R/P294R/ | | | |
| | | E300R/I393L/N452R/ | | | |
| | | I470S/Q483K/F509R/I584V | | | |
| 291/292 | E253R/E300R/ | S36T/E52G/G101A/I124L/ | +++ | ++ | + |
| | N454G/P456T/ | I212V/A241Q/E253R/P294R/ | | | |
| | R545K | E300R/I393L/N452R/ | | | |
| | | N454G/P456T/I470S/Q483K/ | | | |
| | | F509R/R545K | | | |
| 293/294 | E21M/E253R/ | E21M/S36T/E52G/G101A/ | +++ | ++ | + |
| | E300R/Q573V | I124L/I212V/A241Q/E253R/ | | | |
| | | P294R/E300R/I393L/ | | | |
| | | N452R/I470S/Q483K/F509R/ | | | |
| | | Q573V | | | |
| 295/296 | E300R/N454G/ | S36T/E52G/G101A/I124L/ | ++ | + | + |
| | R545K | I212V/A241Q/P294R/E300R/ | | | |
| | | I393L/N452R/N454G/ | | | |
| | | I470S/Q483K/F509R/R545K | | | |
| 297/298 | N454G/P456T/ | S36T/E52G/G101A/I124L/ | +++ | + | + |
| | D541G/R545K/ | I212V/A241Q/P294R/I393L/ | | | |
| | Q573V/I584V | N452R/N454G/P456T/ | | | |
| | | I470S/Q483K/F509R/ | | | |
| | | D541G/R545K/Q573V/I584V | | | |
| 299/300 | S152R/E253R/ | S36T/E52G/G101A/I124L/ | +++ | ++ | + |
| | N454G/T505H/ | S152R/I212V/A241Q/E253R/ | | | |
| | R545K/Q573V | P294R/I393L/N452R/ | | | |
| | | N454G/I470S/Q483K/T505H/ | | | |
| | | F509R/R545K/Q573V | | | |
| 301/302 | E21M/E300R/ | E21M/S36T/E52G/G101A/ | ++ | + | + |
| | N454G/P456T/ | I124L/I212V/A241Q/P294R/ | | | |
| | L495F | E300R/I393L/N452R/ | | | |
| | | N454G/P456T/I470S/Q483K/ | | | |
| | | L495F/F509R | | | |
| 303/304 | E300R/Q573V | S36T/E52G/G101A/I124L/ | +++ | + | + |
| | | I212V/A241Q/P294R/E300R/ | | | |
| | | I393L/N452R/I470S/ | | | |
| | | Q483K/F509R/Q573V | | | |
| 305/306 | E154W/E300R/ | S36T/E52G/G101A/I124L/ | ++ | ++ | + |
| | N454G/R545K | E154W/I212V/A241Q/ | | | |
| | | P294R/E300R/I393L/ | | | |
| | | N452R/N454G/I470S/Q483K/ | | | |
| | | F509R/R545K | | | |
| 307/308 | E154W/E253R/ | S36T/E52G/G101A/I124L/ | ++ | + | + |
| | E300R/R545K/ | E154W/I212V/A241Q/ | | | |
| | I584V | E253R/P294R/E300R/ | | | |
| | | I393L/N452R/I470S/Q483K/ | | | |
| | | F509R/R545K/I584V | | | |
| 309/310 | E21M/E300R/ | E21M/S36T/E52G/G101A/ | ++ | + | + |
| | T505H | I124L/I212V/A241Q/P294R/ | | | |
| | | E300R/I393L/N452R/ | | | |
| | | I470S/Q483K/T505H/F509R | | | |
| 311/312 | E21M/S152R/ | E21M/S36T/E52G/G101A/ | +++ | + | + |
| | E253R/E300R/ | I124L/S152R/I212V/A241Q/ | | | |
| | N454G/P456T/ | E253R/P294R/E300R/ | | | |
| | R545K | 393L/N452R/N454G/P456T/ | | | |
| | | I470S/Q483K/F509R/R545K | | | |
| 313/314 | E154W/E253R/ | S36T/E52G/G101A/I124L/ | ++ | + | + |
| | E300R/P456T/ | E154W/I212V/A241Q/ | | | |
| | D541R/Q573V | E253R/P294R/E300R/I393L/ | | | |
| | | N452R/P456T/I470S/ | | | |
| | | Q483K/F509R/D541R/Q573V | | | |
| 315/316 | E154W/E300R/ | S36T/E52G/G101A/I124L/ | ++ | + | + |
| | N454G | E154W/I212V/A241Q/ | | | |
| | | P294R/E300R/I393L/N452R/ | | | |
| | | N454G/I470S/Q483K/F509R | | | |
| 317/318 | E253R/E300R/ | S36T/E52G/G101A/I124L/ | +++ | + | + |
| | N454G/D541R | I212V/A241Q/E253R/P294R/ | | | |
| | | E300R/I393L/N452R/ | | | |
| | | N454G/I470S/Q483K/ | | | |
| | | F509R/D541R | | | |
| 319/320 | E253R/N454G/ | S36T/E52G/G101A/I124L/ | ++ | + | + |
| | P456T/D541G/ | I212V/A241Q/E253R/P294R/ | | | |
| | I584V | I393L/N452R/N454G/ | | | |
| | | P456T/I470S/Q483K/ | | | |
| | | F509R/D541G/I584V | | | |
| 321/322 | E21M/E300R/ | E21M/S36T/E52G/G101A/ | +++ | + | + |
| | Q573V | I124L/I212V/A241Q/P294R/ | | | |
| | | E300R/I393L/N452R/ | | | |
| | | I470S/Q483K/F509R/Q573V | | | |
| 323/324 | E300R/N454G/ | S36T/E52G/G101A/I124L/ | ++ | + | + |
| | P456T | I212V/A241Q/P294R/E300R/ | | | |
| | | I393L/N452R/N454G/ | | | |
| | | P456T/I470S/Q483K/F509R | | | |
| 325/326 | E300R/N454G | S36T/E52G/G101A/I124L/ | ++ | + | + |
| | | I212V/A241Q/P294R/E300R/ | | | |
| | | I393L/N452R/N454G/ | | | |
| | | I470S/Q483K/F509R | | | |
| 327/328 | E154W/E253R/ | S36T/E52G/G101A/I124L/ | ++ | + | + |
| | E300R/N454G/ | E154W/I212V/A241Q/ | | | |
| | P456T | E253R/P294R/E300R/I393L/ | | | |
| | | N452R/N454G/P456T/ | | | |
| | | I470S/Q483K/F509R | | | |
| 329/330 | E154W/P456T/ | S36T/E52G/G101A/I124L/ | ++ | + | |
| | T505H/D593S | E154W/I212V/A241Q/ | | | |
| | | P294R/I393L/N452R/P456T/ | | | |
| | | I470S/Q483K/T505H/ | | | |
| | | F509R/D593S | | | |
| 331/332 | N454G/P456T/ | S36T/E52G/G101A/I124L/ | +++ | | |
| | T505H | I212V/A241Q/P294R/I393L/ | | | |
| | | N452R/N454G/P456T/ | | | |
| | | I470S/Q483K/T505H/F509R | | | |
| 333/334 | E253R/N454G/ | S36T/E52G/G101A/I124L/ | ++ | | |
| | P456T/I584V | I212V/A241Q/E253R/P294R/ | | | |
| | | I393L/N452R/N454G/ | | | |
| | | P456T/I470S/Q483K/F509R/ | | | |
| | | I584V | | | |
| 335/336 | E154W/E253R/ | S36T/E52G/G101A/I124L/ | ++ | | |
| | E300R | E154W/I212V/A241Q/ | | | |
| | | E253R/P294R/E300R/I393L/ | | | |
| | | N452R/I470S/Q483K/F509R | | | |
| 337/338 | E253R/P456T/ | S36T/E52G/G101A/I124L/ | ++ | | |
| | T505H/I584V | I212V/A241Q/E253R/P294R/ | | | |
| | | I393L/N452R/P456T/ | | | |
| | | I470S/Q483K/T505H/F509R/ | | | |
| | | I584V | | | |
| 339/340 | N454G/P456T | S36T/E52G/G101A/I124L/ | ++ | | |
| | | I212V/A241Q/P294R/I393L/ | | | |
| | | N452R/N454G/P456T/ | | | |
| | | I470S/Q483K/F509R | | | |
| 341/342 | E154W/N454G | S36T/E52G/G101A/I124L/ | + | | |
| | | E154W/I212V/A241Q/ | | | |
| | | P294R/I393L/N452R/N454G/ | | | |
| | | I470S/Q483K/F509R | | | |
| 343/344 | I584V | S36T/E52G/G101A/I124L/ | + | | |
| | | I212V/A241Q/P294R/I393L/ | | | |
| | | N452R/I470S/Q483K/ | | | |
| | | F509R/I584V | | | |
| 345/346 | Q432K/T505H/ | S36T/E52G/G101A/I124L/ | + | | |
| | Q573V | I212V/A241Q/P294R/I393L/ | | | |
| | | Q432K/N452R/I470S/ | | | |
| | | Q483K/T505H/F509R/Q573V | | | |
| 347/348 | P456T/I584V | S36T/E52G/G101A/I124L/ | + | | |
| | | I212V/A241Q/P294R/I393L/ | | | |
| | | N452R/P456T/I470S/ | | | |
| | | Q483K/F509R/I584V | | | |
| 349/350 | E21M/T505H/ | E21M/S36T/E52G/G101A/ | + | | |
| | R545K/I584V | I124L/I212V/A241Q/P294R/ | | | |
| | | I393L/N452R/I470S/ | | | |
| | | Q483K/T505H/F509R/ | | | |
| | | R545K/I584V | | | |
| 351/352 | E21V/E154W/ | E21V/S36T/E52G/G101A/ | + | | |
| | R545K/Q573V | I124L/E154W/I212V/A241Q/ | | | |
| | | P294R/I393L/N452R/ | | | |
| | | I470S/Q483K/F509R/ | | | |
| | | R545K/Q573V | | | |
| 353/354 | N454G/I584V | S36T/E52G/G101A/I124L/ | + | | |
| | | I212V/A241Q/P294R/I393L/ | | | |
| | | N452R/N454G/I470S/ | | | |
| | | Q483K/F509R/I584V | | | |
| 355/356 | E154W/E253R/ | S36T/E52G/G101A/I124L/ | + | | |
| | T505H | E154W/I212V/A241Q/ | | | |
| | | E253R/P294R/I393L/N452R/ | | | |
| | | I470S/Q483K/T505H/F509R | | | |
| 357/358 | T505H | S36T/E52G/G101A/I124L/ | | | |
| | | I212V/A241Q/P294R/I393L/ | | | |
| | | N452R/I470S/Q483K/ | | | |
| | | T505H/F509R | | | |

¹ Levels of increased activity following temperature challenges at 77 °C, 78 °C, and 79 °C were determined relative to the reference polypeptide of SEQ ID NO: 80 and defined as follows: “+” 1.12 to 3.50, “++” > 3.50, “+++” > 4.75

### Example 8

### Improvements Over SEQ ID NO: 224 in Isothermal Polymerase Activity

SEQ ID NO: 224 was selected as the parent enzyme for this round of directed evolution. Libraries of engineered genes were produced using established techniques (e.g., saturation mutagenesis, recombination of previously identified beneficial mutations). The polypeptides encoded by each gene were produced in HTP as described in Example 2, and the soluble lysate was generated as described in Example 3 with replicate plates lysed with lysis temperatures of 75 °C. The clarified lysate was diluted eight-fold prior to setting up the RT-LAMP reactions with 100 copies SARS-CoV-2 RNA as described in Example 4.

Residual activity after heat treatment relative to SEQ ID NO: 224 (activity FIOP) was calculated as the inverse of the quantification cycle (Cq) value and is shown in Table 8.1. Samples which do not have detectable signal above the threshold value after 120 cycles (including positive and negative controls with no detectable signal) are set to a Cq of 120 (the maximum cycles measured) rather than not detected (n.d.) to enable fold-improvement calculations.

TABLE 8.1
Activity of Variants Relative to SEQ ID NO: 224

| | Amino Acid | Amino Acid | Activity FIOP ¹ |
|---|---|---|---|
| SEQ ID | Differences | Differences | (Relative to |
| NO: | (Relative to | (Relative to | SEQ ID NO: |
| (nt/aa) | SEQ ID NO: 224) | SEQ ID NO: 2) | 224) |
| 359/360 | T144R/E154W/T505H/ | S36T/E52G/G101A/I124L/T144R/E154W/I212V/ | +++ |
| | K547H/Q573V/ | A241Q/E253R/P294R/E300R/I393L/N452R/ | |
| | V584I | N454G/P456T/I470S/Q483K/T505H/F509R/ | |
| | | K547H/Q573V | |
| 361/362 | E154W/H191E/R325W/ | S36T/E52G/G101A/I124L/E154W/H191E/I212V/ | +++ |
| | T505H/Q573V/ | A241Q/E253R/P294R/E300R/R325W/I393L/ | |
| | V584I | N452R/N454G/P456T/I470S/Q483K/T505H/ | |
| | | F509R/Q573V | |
| 363/364 | T144R/E154W/P373A/ | S36T/E52G/G101A/I124L/T144R/E154W/I212V/ | +++ |
| | D374E/T505H/ | A241Q/E253R/P294R/E300R/P373A/D374E/ | |
| | Q573V/V584I | I393L/N452R/N454G/P456T/I470S/Q483K/ | |
| | | T505H/F509R/Q573V | |
| 365/366 | S134R/E136P/E154W/ | S36T/E52G/G101A/I124L/S134R/E136P/E154W/ | +++ |
| | T505H/K547H/Q573V/ | I212V/A241Q/E253R/P294R/E300R/I393L/ | |
| | V584I | N452R/N454G/P456T/I470S/Q483K/T505H/ | |
| | | F509R/K547H/Q573V | |
| 367/368 | S81R/T144R/E154W/ | S36T/E52G/S81R/G101A/I124L/T144R/E154W/ | +++ |
| | T505H/K547H/Q573V/ | I212V/A241Q/E253R/P294R/E300R/I393L/ | |
| | V584I | N452R/N454G/P456T/I470S/Q483K/T505H/ | |
| | | F509R/K547H/Q573V | |
| 225/226 | E154W/T505H/ | S36T/E52G/G101A/I124L/E154W/I212V/A241Q/ | ++ |
| | Q573V/V584I | E253R/P294R/E300R/I393L/N452R/N454G/ | |
| | | P456T/I470S/Q483K/T505H/F509R/Q573V | |
| 369/370 | T144R/E154W/H191R/ | S36T/E52G/G101A/I124L/T144R/E154W/H191R/ | ++ |
| | E230G/V322C/ | I212V/E230G/A241Q/E253R/P294R/E300R/ | |
| | T505H/Q573V/ | V322C/I393L/N452R/N454G/P456T/I470S/ | |
| | V584I | Q483K/T505H/F509R/Q573V | |
| 371/372 | E68G/T144R/E154W/ | S36T/E52G/E68G/G101A/I124L/T144R/E154W/ | ++ |
| | T505H/Q573V/V584I | I212V/A241Q/E253R/P294R/E300R/I393L/ | |
| | | N452R/N454G/P456T/I470S/Q483K/T505H/ | |
| | | F509R/Q573V | |
| 373/374 | T144R/E154W/E226K/ | S36T/E52G/G101A/I124L/T144R/E154W/I212V/ | ++ |
| | E230Q/T505H/ | E226K/E230Q/A241Q/E253R/P294R/E300R/ | |
| | Q573V/V584I | I393L/N452R/N454G/P456T/I470S/Q483K/ | |
| | | T505H/F509R/Q573V | |
| 375/376 | T144R/E154W/D374E/ | S36T/E52G/G101A/I124L/T144R/E154W/I212V/ | ++ |
| | K486R/T505H/ | A241Q/E253R/P294R/E300R/D374E/I393L/ | |
| | Q573V/V584I | N452R/N454G/P456T/I470S/Q483K/K486R/ | |
| | | T505H/F509R/Q573V | |
| 377/378 | S81R/E114S/T144R/ | S36T/E52G/S81R/G101A/E114S/I124L/T144R/ | ++ |
| | E154W/T505H/ | E154W/I212V/A241Q/E253R/P294R/E300R/ | |
| | Q573V/V584I | I393L/N452R/N454G/P456T/I470S/Q483K/ | |
| | | T505H/F509R/Q573V | |
| 379/380 | T144R/E154W/ | S36T/E52G/G101A/I124L/T144R/E154W/I212V/ | ++ |
| | K276G/T505H/Q573V/ | A241Q/E253R/K276G/P294R/E300R/I393L/ | |
| | V584I | N452R/N454G/P456T/I470S/Q483K/T505H/ | |
| | | F509R/Q573V | |
| 381/382 | S134T/T144R/ | S36T/E52G/G101A/I124L/S134T/T144R/E154W/ | ++ |
| | E154W/T505H/Q573V/ | I212V/A241Q/E253R/P294R/E300R/I393L/ | |
| | V584I | N452R/N454G/P456T/I470S/Q483K/T505H/ | |
| | | F509R/Q573V | |
| 383/384 | T144R/E154W/ | S36T/E52G/G101A/I124L/T144R/E154W/I212V/ | + |
| | T505H/Q573V/V584I | A241Q/E253R/P294R/E300R/I393L/N452R/ | |
| | | N454G/P456T/I470S/Q483K/T505H/F509R/ | |
| | | Q573V | |
| 385/386 | S81R/T144R/E154W/ | S36T/E52G/S81R/G101A/I124L/T144R/E154W/ | + |
| | R183P/T505H/K547A/ | R183P/I212V/A241Q/E253R/P294R/E300R/ | |
| | Q573V/V584I | 393L/N452R/N454G/P456T/I470S/Q483K/ | |
| | | T505H/F509R/K547A/Q573V | |
| 387/388 | E68G/S81R/Q133A/ | S36T/E52G/E68G/S81R/G101A/I124L/Q133A/ | + |
| | S134R/T144R/E154W/ | S134R/T144R/E154W/I212V/A241Q/E253R/ | |
| | T505H/K547H/ | P294R/E300R/I393L/N452R/N454G/P456T/ | |
| | Q573V/V584I | I470S/Q483K/T505H/F509R/K547H/Q573V | |
| 389/390 | T144R/E154W/D374E/ | S36T/E52G/G101A/I124L/T144R/E154W/I212V/ | + |
| | T505H/Q573V/ | A241Q/E253R/P294R/E300R/D374E/I393L/ | |
| | V584I | N452R/N454G/P456T/I470S/Q483K/T505H/ | |
| | | F509R/Q573V | |
| 391/392 | E92G/Q133A/T144R/ | S36T/E52G/E92G/G101A/I124L/Q133A/T144R/ | + |
| | E154W/T505H/ | E154W/I212V/A241Q/E253R/P294R/E300R/ | |
| | Q573V/V584I | I393L/N452R/N454G/P456T/I470S/Q483K/ | |
| | | T505H/F509R/Q573V | |
| 393/394 | T144R/E154W/P373A/ | S36T/E52G/G101A/I124L/T144R/E154W/I212V/ | + |
| | T505H/Q573V/ | A241Q/E253R/P294R/E300R/P373A/I393L/ | |
| | V584I | N452R/N454G/P456T/I470S/Q483K/T505H/ | |
| | | F509R/Q573V | |
| 395/396 | E114S/T144R/E154W/ | S36T/E52G/G101A/E114S/I124L/T144R/E154W/ | + |
| | T505H/Q573V/ | I212V/A241Q/E253R/P294R/E300R/I393L/ | |
| | V584I | N452R/N454G/P456T/I470S/Q483K/T505H/ | |
| | | F509R/Q573V | |
| 397/398 | E154W/T505H/R551K/ | S36T/E52G/G101A/I124L/E154W/I212V/A241Q/ | + |
| | Q573R/V584I/ | E253R/P294R/E300R/I393L/N452R/N454G/ | |
| | Q585R | P456T/I470S/Q483K/T505H/F509R/R551K/ | |
| | | Q573R/Q585R | |
| 399/400 | S81R/E154W/T505H/ | S36T/E52G/S81R/G101A/I124L/E154W/I212V/ | + |
| | K547V/Q573V/ | A241Q/E253R/P294R/E300R/I393L/N452R/ | |
| | V584I | N454G/P456T/I470S/Q483K/T505H/F509R/ | |
| | | K547V/Q573V | |

¹ Levels of increased activity were determined relative to the reference polypeptide of SEQ ID NO: 224 and defined as follows: “+” 1.05 to 1.15, “++” > 1.15, “+++” > 1.25

### Example 9

### Improvements Over SEQ ID NO: 366 in Isothermal Polymerase Activity

SEQ ID NO: 366 was selected as the parent enzyme for this round of directed evolution. Libraries of engineered genes were produced using well established techniques (e.g., saturation mutagenesis, recombination of previously identified beneficial mutations). The polypeptides encoded by each gene were produced in HTP as described in Example 2, and the soluble lysate was generated as described in Example 3 with replicate plates lysed with a lysis temperature of 80 °C. The clarified lysate was diluted eight-fold prior to setting up the RT-LAMP reactions with 500 copies SARS-CoV-2 RNA as described in Example 4.

Residual activity after heat treatment relative to SEQ ID NO: 366 (activity FIOP) was calculated as the inverse of the quantification cycle (Cq) value and is shown in Table 9.1. Samples which do not have detectable signal above the threshold value after 120 cycles (including positive and negative controls with no detectable signal) are set to a Cq of 120 (the maximum cycles measured) rather than not detected (n.d.) to enable fold-improvement calculations.

TABLE 9.1
Activity of Variants Relative to SEQ ID NO: 366

| | Amino Acid | Amino Acid | Activity FIOP ¹ |
|---|---|---|---|
| SEQ ID | Differences | Differences | (Relative to |
| NO: | (Relative to | (Relative to | SEQ ID NO: |
| (nt/aa) | SEQ ID NO: 366) | SEQ ID NO: 2) | 366) |
| 401/402 | L24M/A58K/ | L24M/S36T/E52G/A58K/G101A/I124L/S134R/ | +++ |
| | Q432L | E136P/E154W/I212V/A241Q/E253R/P294R/E300R/ | |
| | | I393L/Q432L/N452R/N454G/P456T/I470S/ | |
| | | Q483K/T505H/F509R/K547H/Q573V | |
| 403/404 | A58K/Q432L/ | S36T/E52G/A58K/G101A/I124L/S134R/E136P/ | +++ |
| | I575V | E154W/I212V/A241Q/E253R/P294R/E300R/I393L/ | |
| | | Q432L/N452R/N454G/P456T/I470S/Q483K/ | |
| | | T505H/F509R/K547H/Q573V/I575V | |
| 405/406 | A58K/L115Y/ | S36T/E52G/A58K/G101A/L115Y/I124L/S134R/ | +++ |
| | Q432L/I575V | E136P/E154W/I212V/A241Q/E253R/P294R/E300R/ | |
| | | I393L/Q432L/N452R/N454G/P456T/I470S/ | |
| | | Q483K/T505H/F509R/K547H/Q573V/I575V | |
| 407/408 | L24M/L115Y/ | L24M/S36T/E52G/G101A/L115Y/I124L/S134R/ | +++ |
| | Q432L/I575V | E136P/E154W/I212V/A241Q/E253R/P294R/E300R/ | |
| | | I393L/Q432L/N452R/N454G/P456T/I470S/ | |
| | | Q483K/T505H/F509R/K547H/Q573V/I575V | |
| 409/410 | L24M/P25L/ | L24M/P25L/S36T/E52G/G101A/I124L/S134R/ | +++ |
| | V221M/Q432L | E136P/E154W/I212V/V221M/A241Q/E253R/ | |
| | | P294R/E300R/I393L/Q432L/N452R/N454G/P456T/ | |
| | | I470S/Q483K/T505H/F509R/K547H/Q573V | |
| 411/412 | P25L/A58K/ | P25L/S36T/E52G/A58K/G101A/I124L/S134R/ | +++ |
| | Q432L/I575V | E136P/E154W/I212V/A241Q/E253R/P294R/E300R/ | |
| | | I393L/Q432L/N452R/N454G/P456T/I470S/ | |
| | | Q483K/T505H/F509R/K547H/Q573V/I575V | |
| 413/414 | L24M/P25M/A58K/ | L24M/P25M/S36T/E52G/A58K/G101A/I124L/ | ++ |
| | Q432L/I575V | S134R/E136P/E154W/I212V/A241Q/E253R/ | |
| | | P294R/E300R/I393L/Q432L/N452R/N454G/P456T/ | |
| | | I470S/Q483K/T505H/F509R/K547H/Q573V/I575V | |
| 415/416 | V221M/Q432L/ | S36T/E52G/G101A/I124L/S134R/E136P/E154W/ | ++ |
| | I575V | I212V/V221M/A241Q/E253R/P294R/E300R/I393L/ | |
| | | Q432L/N452R/N454G/P456T/I470S/Q483K/ | |
| | | T505H/F509R/K547H/Q573V/I575V | |
| 417/418 | Q432L/I575V | S36T/E52G/G101A/I124L/S134R/E136P/E154W/ | ++ |
| | | I212V/A241Q/E253R/P294R/E300R/I393L/Q432L/ | |
| | | N452R/N454G/P456T/I470S/Q483K/T505H/ | |
| | | F509R/K547H/Q573V/I575V | |
| 419/420 | L24M/V221M/ | L24M/S36T/E52G/G101A/I124L/S134R/E136P/ | ++ |
| | Q432L | E154W/I212V/V221M/A241Q/E253R/P294R/E300R/ | |
| | | I393L/Q432L/N452R/N454G/P456T/I470S/ | |
| | | Q483K/T505H/F509R/K547H/Q573V | |
| 421/422 | L24M/L115Y/ | L24M/S36T/E52G/G101A/L115Y/I124L/S134R/ | ++ |
| | V221M/Q432L | E136P/E154W/I212V/V221M/A241Q/E253R/P294R/ | |
| | | E300R/I393L/Q432L/N452R/N454G/P456T/ | |
| | | 470S/Q483K/T505H/F509R/K547H/Q573V | |
| 423/424 | L24M/P25M/ | L24M/P25M/S36T/E52G/G101A/I124L/S134R/ | ++ |
| | V221M/Q432L | E136P/E154W/I212V/V221M/A241Q/E253R/P294R/ | |
| | | E300R/I393L/Q432L/N452R/N454G/P456T/ | |
| | | 470S/Q483K/T505H/F509R/K547H/Q573V | |
| 425/426 | L24M/L115Y/V221M/ | L24M/S36T/E52G/G101A/L115Y/I124L/S134R/ | ++ |
| | Q432L/I575V | E136P/E154W/I212V/V221M/A241Q/E253R/P294R/ | |
| | | E300R/I393L/Q432L/N452R/N454G/P456T/ | |
| | | I470S/Q483K/T505H/F509R/K547H/Q573V/I575V | |
| 427/428 | Q432L | S36T/E52G/G101A/I124L/S134R/E136P/E154W/ | ++ |
| | | 212V/A241Q/E253R/P294R/E300R/I393L/Q432L/ | |
| | | N452R/N454G/P456T/I470S/Q483K/T505H/ | |
| | | F509R/K547H/Q573V | |
| 429/430 | L24M/E34D/Q432L | L24M/E34D/S36T/E52G/G101A/I124L/S134R/ | ++ |
| | | E136P/E154W/I212V/A241Q/E253R/P294R/E300R/ | |
| | | I393L/Q432L/N452R/N454G/P456T/I470S/Q483K/ | |
| | | T505H/F509R/K547H/Q573V | |
| 431/432 | L24M/Q432L/I575V | L24M/S36T/E52G/G101A/I124L/S134R/E136P/ | ++ |
| | | E154W/I212V/A241Q/E253R/P294R/E300R/I393L/ | |
| | | Q432L/N452R/N454G/P456T/I470S/Q483K/ | |
| | | T505H/F509R/K547H/Q573V/I575V | |
| 433/434 | A184T/V221M/ | S36T/E52G/G101A/I124L/S134R/E136P/E154W/ | ++ |
| | Q432L/I575V | A184T/I212V/V221M/A241Q/E253R/P294R/E300R/ | |
| | | I393L/Q432L/N452R/N454G/P456T/I470S/ | |
| | | Q483K/T505H/F509R/K547H/Q573V/I575V | |
| 435/436 | L24M/Q432L | L24M/S36T/E52G/G101A/I124L/S134R/E136P/ | ++ |
| | | E154W/I212V/A241Q/E253R/P294R/E300R/I393L/ | |
| | | Q432L/N452R/N454G/P456T/I470S/Q483K/ | |
| | | T505H/F509R/K547H/Q573V | |
| 437/438 | L24M/P25L/A58K/ | L24M/P25L/S36T/E52G/A58K/G101A/L115Y/ | ++ |
| | L115Y/Q432L/ | I124L/S134R/E136P/E154W/I212V/A241Q/E253R/ | |
| | I575V | P294R/E300R/I393L/Q432L/N452R/N454G/ | |
| | | P456T/I470S/Q483K/T505H/F509R/K547H/ | |
| | | Q573V/I575V | |
| 439/440 | P25L/V221M/ | P25L/S36T/E52G/G101A/I124L/S134R/E136P/ | ++ |
| | Q432L | E154W/I212V/V221M/A241Q/E253R/P294R/E300R/ | |
| | | I393L/Q432L/N452R/N454G/P456T/I470S/ | |
| | | Q483K/T505H/F509R/K547H/Q573V | |
| 441/442 | P25M/A58K/ | P25M/S36T/E52G/A58K/G101A/L115Y/I124L/ | ++ |
| | L115Y/Q432L | S134R/E136P/E154W/I212V/A241Q/E253R/P294R/ | |
| | | E300R/I393L/Q432L/N452R/N454G/P456T/ | |
| | | I470S/Q483K/T505H/F509R/K547H/Q573V | |
| 443/444 | V221M/I575V | S36T/E52G/G101A/I124L/S134R/E136P/E154W/ | ++ |
| | | 212V/V221M/A241Q/E253R/P294R/E300R/I393L/ | |
| | | N452R/N454G/P456T/I470S/Q483K/T505H/ | |
| | | F509R/K547H/Q573V/I575V | |
| 445/446 | V221M/Q432L | S36T/E52G/G101A/I124L/S134R/E136P/E154W/ | ++ |
| | | 212V/V221M/A241Q/E253R/P294R/E300R/I393L/ | |
| | | Q432L/N452R/N454G/P456T/I470S/Q483K/ | |
| | | T505H/F509R/K547H/Q573V | |
| 447/448 | L24M/A58K/L115Y/ | L24M/S36T/E52G/A58K/G101A/L115Y/I124L/ | ++ |
| | V221M/I575V | S134R/E136P/E154W/I212V/V221M/A241Q/E253R/ | |
| | | P294R/E300R/I393L/N452R/N454G/P456T/ | |
| | | I470S/Q483K/T505H/F509R/K547H/Q573V/I575V | |
| 449/450 | L24M/P25M/V221M/ | L24M/P25M/S36T/E52G/G101A/I124L/S134R/ | ++ |
| | Q432L/I575V | E136P/E154W/I212V/V221M/A241Q/E253R/P294R/ | |
| | | E300R/I393L/Q432L/N452R/N454G/P456T/ | |
| | | 470S/Q483K/T505H/F509R/K547H/Q573V/I575V | |
| 451/452 | L24M/P25M/A58K/ | L24M/P25M/S36T/E52G/A58K/G101A/I124L/S134R/ | ++ |
| | V221M/Q432L | E136P/E154W/I212V/V221M/A241Q/E253R/ | |
| | | P294R/E300R/I393L/Q432L/N452R/N454G/ | |
| | | P456T/I470S/Q483K/T505H/F509R/K547H/Q573V | |
| 453/454 | E34D/A58K/I105S/ | E34D/S36T/E52G/A58K/G101A/I105S/I124L/ | ++ |
| | Q432L | S134R/E136P/E154W/I212V/A241Q/E253R/P294R/ | |
| | | E300R/I393L/Q432L/N452R/N454G/P456T/I470S/ | |
| | | Q483K/T505H/F509R/K547H/Q573V | |
| 455/456 | L24M/P25M/ | L24M/P25M/S36T/E52G/G101A/L115Y/I124L/ | ++ |
| | L115Y/Q432L | S134R/E136P/E154W/I212V/A241Q/E253R/P294R/ | |
| | | E300R/I393L/Q432L/N452R/N454G/P456T/ | |
| | | I470S/Q483K/T505H/F509R/K547H/Q573V | |
| 457/458 | L24M/V221M/ | L24M/S36T/E52G/G101A/I124L/S134R/E136P/ | + |
| | I575V | E154W/I212V/V221M/A241Q/E253R/P294R/E300R/ | |
| | | I393L/N452R/N454G/P456T/I470S/Q483K/ | |
| | | T505H/F509R/K547H/Q573V/I575V | |
| 459/460 | L115Y/Q432L | S36T/E52G/G101A/L115Y/I124L/S134R/E136P/ | + |
| | | E154W/I212V/A241Q/E253R/P294R/E300R/I393L/ | |
| | | Q432L/N452R/N454G/P456T/I470S/Q483K/ | |
| | | T505H/F509R/K547H/Q573V | |
| 461/462 | A58K/V221M | S36T/E52G/A58K/G101A/I124L/S134R/E136P/ | + |
| | | E154W/I212V/V221M/A241Q/E253R/P294R/E300R/ | |
| | | I393L/N452R/N454G/P456T/I470S/Q483K/ | |
| | | T505H/F509R/K547H/Q573V | |
| 463/464 | A58K/I575V | S36T/E52G/A58K/G101A/I124L/S134R/E136P/ | + |
| | | E154W/I212V/A241Q/E253R/P294R/E300R/I393L/ | |
| | | N452R/N454G/P456T/I470S/Q483K/T505H/ | |
| | | F509R/K547H/Q573V/I575V | |
| 465/466 | P25L/Q432L/ | P25L/S36T/E52G/G101A/I124L/S134R/E136P/ | + |
| | I575V | E154W/I212V/A241Q/E253R/P294R/E300R/I393L/ | |
| | | Q432L/N452R/N454G/P456T/I470S/Q483K/T505H/ | |
| | | F509R/K547H/Q573V/I575V | |
| 467/468 | P25L/Q432L | P25L/S36T/E52G/G101A/I124L/S134R/E136P/ | + |
| | | E154W/I212V/A241Q/E253R/P294R/E300R/I393L/ | |
| | | Q432L/N452R/N454G/P456T/I470S/Q483K/T505H/ | |
| | | F509R/K547H/Q573V | |
| 469/470 | L24M/V221M/ | L24M/S36T/E52G/G101A/I124L/S134R/E136P/ | + |
| | Q432L/I575V | E154W/I212V/V221M/A241Q/E253R/P294R/E300R/ | |
| | | I393L/Q432L/N452R/N454G/P456T/I470S/ | |
| | | Q483K/T505H/F509R/K547H/Q573V/I575V | |
| 471/472 | L24M/L115Y/ | L24M/S36T/E52G/G101A/L115Y/I124L/S134R/ | + |
| | Q432L | E136P/E154W/I212V/A241Q/E253R/P294R/E300R/ | |
| | | I393L/Q432L/N452R/N454G/P456T/I470S/Q483K/ | |
| | | T505H/F509R/K547H/Q573V | |
| 473/474 | L24M/P25M/I105S/ | L24M/P25M/S36T/E52G/G101A/I105S/I124L/ | + |
| | Q432L | S134R/E136P/E154W/I212V/A241Q/E253R/ | |
| | | P294R/E300R/I393L/Q432L/N452R/N454G/P456T/ | |
| | | I470S/Q483K/T505H/F509R/K547H/Q573V | |
| 475/476 | P25M/E34D/Q432L | P25M/E34D/S36T/E52G/G101A/I124L/S134R/ | + |
| | | E136P/E154W/I212V/A241Q/E253R/P294R/E300R/ | |
| | | I393L/Q432L/N452R/N454G/P456T/I470S/Q483K/ | |
| | | T505H/F509R/K547H/Q573V | |
| 477/478 | L24M/P25L/I105S/ | L24M/P25L/S36T/E52G/G101A/I105S/I124L/ | + |
| | Q432L | S134R/E136P/E154W/I212V/A241Q/E253R/P294R/ | |
| | | E300R/I393L/Q432L/N452R/N454G/P456T/I470S/ | |
| | | Q483K/T505H/F509R/K547H/Q573V | |
| 479/480 | P25M/Q432L | P25M/S36T/E52G/G101A/I124L/S134R/E136P/ | + |
| | | E154W/I212V/A241Q/E253R/P294R/E300R/ | |
| | | I393L/Q432L/N452R/N454G/P456T/I470S/Q483K/ | |
| | | T505H/F509R/K547H/Q573V | |
| 481/482 | P25M/I105S/ | P25M/S36T/E52G/G101A/I105S/I124L/S134R/ | + |
| | V221M/Q432L | E136P/E154W/I212V/V221M/A241Q/E253R/P294R/ | |
| | | E300R/I393L/Q432L/N452R/N454G/P456T/I470S/ | |
| | | Q483K/T505H/F509R/K547H/Q573V | |
| 483/484 | L24M/P25L/Q432L | L24M/P25L/S36T/E52G/G101A/I124L/S134R/ | + |
| | | E136P/E154W/I212V/A241Q/E253R/P294R/E300R/ | |
| | | I393L/Q432L/N452R/N454G/P456T/I470S/Q483K/ | |
| | | T505H/F509R/K547H/Q573V | |
| 485/486 | L24M/A58K/I575V | L24M/S36T/E52G/A58K/G101A/I124L/S134R/ | + |
| | | E136P/E154W/I212V/A241Q/E253R/P294R/E300R/ | |
| | | I393L/N452R/N454G/P456T/I470S/Q483K/ | |
| | | T505H/F509R/K547H/Q573V/I575V | |
| 487/488 | L24M/P25M/I105S/ | L24M/P25M/S36T/E52G/G101A/I105S/I124L/ | + |
| | Q432L/I575V | S134R/E136P/E154W/I212V/A241Q/E253R/P294R/ | |
| | | E300R/I393L/Q432L/N452R/N454G/P456T/I470S/ | |
| | | Q483K/T505H/F509R/K547H/Q573V/I575V | |

¹ Levels of increased activity were determined relative to the reference polypeptide of SEQ ID NO: 366 and defined as follows: “+” 1.13 to 2.00, “++” > 2.00, “+++” > 3.00

### Example 10

### DNA Polymerases for Initial Activity Screening; Purified DNA Polymerase Activity in RT-LAMP Reactions for SARS-CoV-2 Detection

### Shake-Flask Expression

Selected cultures grown as described above were plated onto LB agar plates with 1% glucose and 30 μg/ml chloramphenicol and grown overnight at 30 °C. A single colony from each culture was transferred to 5 ml of LB broth with 1% glucose and 30 μg/ml chloramphenicol. The cultures were grown for 20 h at 30 °C, 250 rpm, and sub-cultured at a dilution of approximately 1:50 into 250 ml of Terrific Broth with 30 μg/ml of chloramphenicol, to a final OD₆₀₀ of about 0.05. The cultures were incubated for approximately 195 min at 30 °C, 250 rpm, to an OD₆₀₀ of about 0.6, and then induced with the addition of IPTG at a final concentration of 1 mM. The induced cultures were incubated for 20 h at 30 °C, 250 rpm. Following this incubation period, the cultures were centrifuged at 4000 rpm×10 min. The culture supernatant was discarded, and the pellets were resuspended in 30 ml of 50 mM Tris-HCl, pH 8. This cell suspension was chilled in an ice bath and lysed using a Microfluidizer cell disruptor (Microfluidics M-110L). The crude lysate was pelleted by centrifugation (11,000 rpm for 60 min at 4 °C), and the supernatant was then filtered through a 0.2 μm PES membrane to further clarify the lysate.

Purification of DNA Polymerase from Shake-Flask Lysates

DNA polymerase lysates were purified using an AKTA Pure purification system and a 5 ml HisTrap FF column (GE Healthcare); the run parameters are provided in Table 10.1. The shake-flask wash buffer comprised 50 mM Tris-HCl, pH 8, 500 mM NaCl, 20 mM imidazole, 0.02% v/v Triton X-100 reagent and the shake-flask elution buffer comprised 50 mM Tris-HCl, pH 8, 500 mM NaCl, 250 mM imidazole, 0.02% v/v Triton X-100 reagent.

TABLE 10.1
Purification Parameters

| Parameter | Volume | |
|---|---|---|
| Column volume | 5 | mL |
| Flow rate | 5 | mL/min |
| Pressure limit | 0.3 | MPa |
| Wash Unbound volume | 20 CV = 100 mL | |
| Elution | Isocratic (step) | |
| Elution volume | 5 CV = 25 mL | |
| Fraction volume | 1.5 | mL |

| | |
|---|---|
| Re-equilibration volume | 5 CV = 25 mL |

The four most concentrated 1.5 mL fractions based on UV absorption (A280) were pooled and dialyzed overnight in dialysis buffer (40 mM Tris-HCl, pH 8, 100 mM KCl, 0.1 mM EDTA, and 50% glycerol) in a 3.5K Slide-A-Lyzer™ dialysis cassette (Thermo Fisher) for buffer exchange. DNA polymerase concentrations in the preparations were measured by absorption at 280 nm and normalized to 2 mg mL⁻¹.

RT-LAMP Reactions with Purified Large Fragment of DNA Polymerase

RT-LAMP assays with purified large fragment of DNA polymerases were conducted using a primer set targeting CoV-Orf1a, consisting of six oligonucleotides at different concentrations (final concentrations FIP: 1.6 uM, BIP: 1.6 uM, F3: 0.2 uM, B3: 0.2 uM, loop F: 0.4 uM, loop B: 0.4 uM). Each variant was screened in a 5 uL reaction that comprised SARS-CoV-2 synthetic RNA control 1 (0.5 uL, GenBank/GISAID ID MT007544.1, Twist Biosciences part number 102019, 5000 copies per reaction), 0.5 uL 10× isothermal amplification buffer (NEB catalog #B0537S), 0.5 uL 10× Orf1a LAMP primer mixture (FIP: 16 uM, BIP: 16 uM, F3: 2 uM, B3: 2 uM, loop F: 4 uM, loop B: 4 uM), dNTP mix (final concentration 1.1 mM each dNTP), 0.3 uL 100 mM MgSO₄ (final concentration 6 mM; 8 mM total including contribution from 10× isothermal amplification buffer), 0.25 uL Evagreen (20× in water stock, 1.25 uM final concentration), 1 uL purified DNA polymerase (final concentration 10 ng uL-1). Synthetic RNA control was added last to initiate the reaction. No-template controls (NTCs) had nuclease-free water added in place of synthetic RNA control solution. The 384-well PCR plate was briefly vortexed and centrifuged to mix, then inserted into a CFX Touch 384-well Real-Time PCR Detection System (Bio-Rad). The isothermal amplification reaction was performed at 55 °C for 120 cycles of 30 see, with the reactions' fluorescence analyzed in the FAM channel after every cycle.

RT-LAMP activity with DNA polymerase in the absence of a separate reverse transcriptase calculated as the ratio of the quantification cycle (Cq) value of the NTC to the Cq of the sample containing 5000 copies of synthetic RNA control and is shown in Table 10.2. Samples which do not have detectable signal above the threshold value after 120 cycles (including positive and negative controls with no detectable signal) are set to a Cq of 120 (the maximum cycles measured) rather than not detected (n.d.) to enable calculation of fold improvements.

TABLE 10.2
Activity of Large Fragment of DNA Polymerase Homologs

| SEQ ID | | | NTC/positive |
|---|---|---|---|
| NO: (nt/aa) | UniProt ID | Source organism | ratio ¹ |
| 1/2 | A0A023CMU9 | Parageobacillus | +++ |
| | | genomosp. 1 | |
| 489/490 | P52026 | Geobacillus | +++ |
| | | stearothermophilus | |
| 491/492 | A0A167UH07 | Anoxybacillus sp. | +++ |
| | | B7M1 | |
| 493/494 | M8D3Y0 | Anoxybacillus | +++ |
| | | flavithermus AK1 | |
| 495/496 | A0A0A2SK72 | Anoxybacillus gonensis | ++ |
| 497/498 | Q08IE4 | Bacillus smithii | ++ |
| 499/500 | A0A4R1QH44 | Thermolongibacillus | ++ |
| | | altinsuensis | |
| 501/502 | Q45458 | Geobacillus | ++ |
| | | stearothermophilus | |
| 503/504 | A0A0B4SB30 | Bacillus sp. WP8 | + |
| 505/506 | A0A0D1JLC4 | Bacillus subtilis | + |
| 507/508 | A0A0N0I8N0 | Geobacillus sp. BCO2 | + |
| 509/510 | A0A3R9UCK4 | Bacillus sp. HMF5848 | + |
| 511/512 | A0A084GX94 | Metabacillus indicus | + |
| | | (Bacillus indicus) | |
| 513/514 | A0A176JAP1 | Bacillus sp. SJS | + |
| 515/516 | A0A1W1II73 | Trichococcus pasteurii | + |
| 517/518 | A0A2S0U8D5 | Paenibacillus sp. | + |
| | | CAA11 | |
| 519/520 | A0A1I5VYY5 | Desemzia incerta | + |
| 521/522 | D5DMV6 | Priestia megaterium | + |
| 523/524 | E6U0L1 | Evansella cellulosilytica | + |
| 525/526 | G8PDR9 | Pediococcus claussenii | + |
| 527/528 | K1KNJ5 | Solibacillus isronensis | + |
| | | B3W22 | |
| 529/530 | L5N8Z2 | Halobacillus sp. BAB- | + |
| | | 2008 | |
| 531/532 | Q03RJ7 | Levilactobacillus brevis | + |

¹ Levels of increased activity were determined relative to the no-template control (NTC) for the same SEQ ID NO and are defined as followed: “+” 1.00 to 1.14, “++” > 1.14, “+++” > 1.48

### Example 11

### Assaying Engineered DNA Polymerase for Resistance to Inhibitors

The resistance of engineered DNA polymerase to inhibitors was examined by reverse transcriptase loop-mediated amplification (RT-LAMP). The reactions targeting 75 copies of synthetic Influenza B RNA and containing EvaGreen® fluorescent dye were performed in 20 μL volumes (n=3) with the addition of each of the following inhibitors at their respective final concentrations: 3% (v/v) ethanol, 25 mM guanidine thiocyanate (GTC), 20% (v/v) viral transport medium (VTM), 6.25 U/mL heparin, 10 μM hematin, 50 ng of human total RNA, or 100 ng of human genomic DNA (hgDNA). Additional reactions of polymerase without inhibitors were run in parallel for comparison. The reactions were conducted at 65 °C (or 68 °C for LavaLAMP) for 72 minutes using the Bio-Rad CFX Opus thermal cycler (Bio Rad, CA). The time to result (TTR) for each replicate was calculated by multiplying the measured Cq values by the time per cycle. The inhibitor resistance was compared to engineered DNA polymerase without inhibitor, and to DNA polymerase Bst 3.0 (New England Biolabs, Catalog #M0374) and to LGC Lava AMP (LGC Biosearch Technologies).

While the present invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered DNA polymerase, or a functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of SEQ ID NO: 2, 10, 80, 224, or 366, or to a reference sequence corresponding to SEQ ID NO: 2, 10, 80, 224, or 366, wherein the amino acid sequence comprises one or more substitutions relative to the reference sequence corresponding to amino acid residues 12 to 604 of SEQ ID NO: 2, 10, 80, 224, or 366, or relative to the reference sequence corresponding to SEQ ID NO: 2, 10, 80, 224, or 366.

2. The engineered DNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12 to 604 of SEQ ID NO: 2, or to the reference sequence corresponding to SEQ ID NO: 2, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

3. (canceled)

4. The engineered DNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12 to 604 of an even-numbered SEQ ID NO. of SEQ ID NOs: 4-488, or to a reference sequence corresponding to an even-numbered SEQ ID NO. of SEQ ID NOs: 4-488, wherein the amino acid sequence comprises one or more substitutions relative to the reference sequence corresponding to amino acid residues 12 to 604 of SEQ ID NO: 2, or relative to the reference sequence corresponding to SEQ ID NO: 2.

5. The engineered DNA polymerase of claim 1, wherein the amino acid sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192, 210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 2.

6. The engineered DNA polymerase of claim 1, wherein the amino acid sequence of the engineered DNA polymerase comprises at least a substitution or amino acid residue 21M/V, 24M, 25L/M, 34D, 36T, 52G, 58K, 66T, 68G, 81R, 84Q, 92G, 101A, 105S, 114S, 115Y, 124L, 133A, 133R, 134R/T, 136P, 144R, 152R, 154W, 168G, 183P, 184T, 191E/R, 192E, 210A, 212V, 221M, 226K, 230G/Q, 241E/Q, 252T, 253R, 276G, 287R, 290G, 294R, 295K, 300A/G/R, 304L, 322C, 325W, 372E/K, 373A, 374E, 393L, 427A, 432K/L, 452R/S, 454G, 456T, 462R, 470S, 483K, 486R, 495F, 505H, 509R, 541G/R, 541T, 545K, 547A/H/V, 551K, 552L, 573R/V, 575V, 578F, 584V, 585R, or 593S, or combinations thereof, wherein the amino acid positions are relative to the reference sequence corresponding to SEQ ID NO: 2.

7. (canceled)

8. (canceled)

9. The engineered DNA polymerase of claim 1, wherein the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 2.

10. The engineered DNA polymerase of claim 1, wherein the amino acid sequence of the engineered DNA polymerase comprises at least a substitution 36T, 52G, 101A, 124L, 134R/T, 136P, 154W, 212V, 241E/Q, 253R, 294R, 300A/G/R, 372E/K, 393L, 452R/S, 454G, 456T, 470S, 483K, 505H, 509R, 547A/H/V, 573R/V, or 584V, or combinations thereof, wherein the amino acid positions are relative to the reference sequence corresponding to SEQ ID NO: 2.

11-14. (canceled)

15. The engineered DNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of SEQ ID NO: 10, 80, 224, or 366, or to the reference sequence corresponding to SEQ ID NO: 10, 80, 224, or 366, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

16. The engineered DNA polymerase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 4-488, or to the reference sequence corresponding to an even-numbered SEQ ID NO. of SEQ ID NOs: 4-488, wherein the amino acid sequence comprises one or more substitutions relative to the reference sequence corresponding to residues 12 to 604 of SEQ ID NO: 10, 80, 224, or 366, or relative to the reference sequence corresponding to SEQ ID NO: 10, 80, 224, or 366.

17. The engineered DNA polymerase of claim 15, wherein the amino acid sequence of the engineered DNA polymerase comprises at least a substitution at amino acid position 21, 24, 25, 25, 34, 36, 52, 58, 66, 68, 81, 84, 92, 101, 105, 114, 115, 124, 133, 133, 134, 136, 144, 152, 154, 168, 183, 184, 191, 192, 210, 212, 221, 226, 230, 241, 252, 253, 276, 287, 290, 294, 295, 300, 304, 322, 325, 372, 373, 374, 393, 427, 432, 452, 454, 456, 462, 470, 483, 486, 495, 505, 509, 541, 541, 545, 547, 551, 552, 573, 575, 578, 584, 585, or 593, or combinations thereof, wherein the amino acid positions are relative to the reference sequence corresponding to SEQ ID NO: 10, 80, 224, or 366.

18. The engineered DNA polymerase of claim 15, wherein the amino acid sequence of the engineered DNA polymerase comprises a substitution or an amino acid residue 21M/V, 24M, 25L/M, 34D, 36T, 52G, 58K, 66T, 68G, 81R, 84Q, 92G, 101A, 105S, 114S, 115Y, 124L, 133A, 133R, 134R/T, 136P, 144R, 152R, 154W, 168G, 183P, 184T, 191E/R, 192E, 210A, 212V, 221M, 226K, 230G/Q, 241E/Q, 252T, 253R, 276G, 287R, 290G, 294R, 295K, 300A/G/R, 304L, 322C, 325W, 372E/K, 373A, 374E, 393L, 427A, 432K/L, 452R/S, 454G, 456T, 462R, 470S, 483K, 486R, 495F, 505H, 509R, 541G/R, 541T, 545K, 547A/H/V, 551K, 552L, 573R/V, 575V, 578F, 584V/1, 585R, or 593S, or combinations thereof, wherein the amino acid positions are relative to the reference sequence corresponding to SEQ ID NO: 10, 80, 224, or 366.

19. (canceled)

20. (canceled)

21. The engineered DNA polymerase of claim 15, wherein the amino acid sequence of the engineered DNA polymerase comprises at least a substitution at amino acid position 36, 52, 101, 124, 134, 136, 154, 212, 241, 253, 294, 300, 372, 393, 452, 454, 456, 470, 483, 505, 509, 547, 573, or 584, or combinations thereof, wherein the amino acid positions are relative to the reference sequence corresponding to SEQ ID NO: 10, 80, 224, or 366.

22. The engineered DNA polymerase of claim 15, wherein the amino acid sequence of the engineered DNA polymerase comprises at least a substitution or amino acid residue 36T, 52G, 101A, 124L, 134R, 136P, 154W, 212V, 241Q, 253R, 294R, 300R, 372K/E, 393L, 452R, 454G, 456T, 470S, 483K, 505H, 509R, 547H, 573V, or 584I/V, or combinations thereof, wherein the amino acid positions are relative to the reference sequence corresponding to SEQ ID NO: 10, 80, 224, or 366.

23. (canceled)

24. The engineered DNA polymerase of claim 16, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 10-218, or to the reference sequence corresponding to an even-numbered SEQ ID NO. of SEQ ID NOs: 10-218, wherein the amino acid sequence comprises one or more substitutions relative to the reference sequence corresponding to residues 12 to 604 of SEQ ID NO: 10, or relative to the reference sequence corresponding to SEQ ID NO: 10.

25. The engineered DNA polymerase of claim 24, wherein the amino acid sequence of the engineered DNA polymerase comprises at least a substitution set at amino acid positions 52/101/124/212/294/372/393/452/483/509, 52/124/300/393/452, 154/212/294/300/372/393/452/483/509, 52/101/154/294/300/452/509/593, 52/154/212/294/300/393/452, 212/300/393/452/509, 52/101/212/294/300/393/452/483/509/593, 52/124/294/300/452/509, 124/300/393/452/483/509/593, 101/124/212/452/483/509, 52/393/452/509/593, 212/300/452/509/593, 52/452/509/593, 154/212/300/372/452/509/593, 52/124/212/294/393/452/593, 124/452/509, 152/253/287/505/541/573/584, 154/212/300/452, 152/253/287/304/454/545/573/584, 52/124/154/294/300/372/452/509, 294/300/372/452/509/593, 52/212/452, 124/294/393/452, 52/300/509/593, 152/253/287/454/541/584, 52/101/452/509, 52/154/212/300/372/393/509, 21/253/287/454/584, 52/393/452, 52/124/154/294/509, 101/124/212/300/393, 124/294/300/372/452/509/593, 52/124/154/294/483/593, 52/372/393/452/509/593, 154/300/393/452/593, 52/101/294/372/393/452/509, 21/152/253/287/304/454/505, 52/300/393, 101/124/154/294/452/509, 154/212/372/393/509/593, 21/253/287/454/505/541, 287/454/505/584, 52/124/294/300/372/452, 287/505/541/573, 253/505/584, 84/253/287/456/573, 52/372/509/593, 509/593, 154/212/300/372/509, 52/154/294/509/593, 152/295/505/545/573, 253/541/584, 253/295/304/454/462/573/584, 253/454/505/573/584, 154/212/300, 287/454/545/573, 21/152/287/304/454, 152/253/295/304/541/573, 152/253/287/304/454, 152/454/573, 124/294/372/509/593, 152/253/295/454, 154/300/452, 287/304/505, 152/253/454/505, 253/454/505, 21/253/454/505/545, 152/253/287/505, 505/541/584, or 152/253/454/584, wherein the amino acid positions are relative to the reference sequence corresponding to SEQ ID NO: 10.

26. (canceled)

27. (canceled)

28. The engineered DNA polymerase of claim 16, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 220-258, or to the reference sequence corresponding to an even-numbered SEQ ID NO. of SEQ ID NOs: 220-258, wherein the amino acid sequence comprises one or more substitutions relative to the reference sequence corresponding to residues 12 to 604 of SEQ ID NO: 80, or relative to the reference sequence corresponding to SEQ ID NO: 80.

29. The engineered DNA polymerase of claim 28, wherein the amino acid sequence of the engineered DNA polymerase comprises at least a substitution or substitution set at amino acid position(s) 300/454/456/541/584, 21/300/454/545/584, 253/300/454/456/584, 154/253/300/454/456/505/573, 21/154/300/454/456/573, 152/300/454/456/505/584/593, 154/253/300/456/505/545/573/584, 253/300/454/456/505/573, 154/253/300/454/456/541/573/584, 154/300/454/505, 21/253/300/454/456/545/573/593, 168/300/454/456/545/573, 253/300/454/505/573, 154/300/505/545/584, 154/253/300/456/541/573/584, 21/154/300/454/456/505/545, 154/253/300/454/505, 21/154/253/300/454/573, 300/454/505/541/584, 300/454/505/541/573, 154/253/300/454, 154/300/454/573, 300/454/456/545/573, 253/300/454/456, 154/253/300/454/545/573, 21/152/253/300/454/456/541/573, 154/253/300/454/456/573, 152/300/454, 154/253/300/454/505/541/593, 21/154/300/454/573/584, 154/300/456/573/593, 152/253/300/454/545/573, 21/300/456/495/505/541/584, 154/300/454/541/584, 253/300/505/541/573/584, 253/300/584, 253/300/454/456/545, 21/253/300/573, 300/454/545, 454/456/541/545/573/584, 152/253/454/505/545/573, 21/300/454/456/495, 300/573, 154/300/454/545, 154/253/300/545/584, 21/300/505, 21/152/253/300/454/456/545, 154/253/300/456/541/573, 154/300/454, 253/300/454/541, 253/454/456/541/584, 21/300/573, 300/454/456, 300/454, 154/253/300/454/456, 154/456/505/593, 454/456/505, 253/454/456/584, 154/253/300, 253/456/505/584, 454/456, 154/454, 584, 432/505/573, 456/584, 21/505/545/584, 21/154/545/573, 454/584, 154/253/505, or 505, wherein the amino acid positions are relative to the reference sequence corresponding to SEQ ID NO: 80.

30. (canceled)

31. (canceled)

32. The engineered DNA polymerase of claim 16, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 360-400, or to the reference sequence corresponding to an even-numbered SEQ ID NO. of SEQ ID NOs: 360-400, wherein the amino acid sequence comprises one or more substitutions relative to the reference sequence corresponding to residues 12 to 604 of SEQ ID NO: 224, or relative to the reference sequence corresponding to SEQ ID NO: 224.

33. The engineered DNA polymerase of claim 32, wherein the amino acid sequence of the engineered DNA polymerase comprises at least a substitution set at amino acid positions 144/154/505/547/573/584, 154/191/325/505/573/584, 144/154/373/374/505/573/584, 134/136/154/505/547/573/584, 81/144/154/505/547/573/584, 154/505/573/584, 144/154/191/230/322/505/573/584, 68/144/154/505/573/584, 144/154/226/230/505/573/584, 144/154/374/486/505/573/584, 81/114/144/154/505/573/584, 144/154/276/505/573/584, 134/144/154/505/573/584, 144/154/505/573/584, 81/144/154/183/505/547/573/584, 68/81/133/134/144/154/505/547/573/584, 144/154/374/505/573/584, 92/133/144/154/505/573/584, 144/154/373/505/573/584, 114/144/154/505/573/584, 154/505/551/573/584/585, or 81/154/505/547/573/584, wherein the amino acid positions are relative to the reference sequence corresponding to SEQ ID NO: 224.

34. (canceled)

35. (canceled)

36. The engineered DNA polymerase of claim 16, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 604 of an even-numbered SEQ ID NO. of SEQ ID NOs: 402-488, or to the reference sequence corresponding to an even-numbered SEQ ID NO. of SEQ ID NOs: 402-488, wherein the amino acid sequence comprises one or more substitutions relative to the reference sequence corresponding to residues 12 to 604 of SEQ ID NO: 366, or relative to the reference sequence corresponding to SEQ ID NO: 366.

37. The engineered DNA polymerase of claim 36, wherein the amino acid sequence of the engineered DNA polymerase comprises at least a substitution or substitution set at amino acid position(s) 24/58/432, 58/432/575, 58/115/432/575, 24/115/432/575, 24/25/221/432, 25/58/432/575, 24/25/58/432/575, 221/432/575, 432/575, 24/221/432, 24/115/221/432, 24/115/221/432/575, 432, 24/34/432, 24/432/575, 184/221/432/575, 24/432, 24/25/58/115/432/575, 25/221/432, 25/58/115/432, 221/575, 221/432, 24/58/115/221/575, 24/25/221/432/575, 24/25/58/221/432, 34/58/105/432, 24/25/115/432, 24/221/575, 115/432, 58/221, 58/575, 25/432/575, 25/432, 24/221/432/575, 24/115/432, 24/25/105/432, 25/34/432, 25/105/221/432, 24/25/432, 24/58/575, or 24/25/105/432/575, wherein the amino acid positions are relative to the reference sequence corresponding to SEQ ID NO: 366.

38. (canceled)

39. (canceled)

40. The engineered DNA polymerase of claim 1, wherein the amino acid sequence of the engineered DNA polymerase comprises residues 12 to 604 of an even-numbered SEQ ID NO. of SEQ ID NOs: 4-488, or comprises an even-numbered SEQ ID NO. of SEQ ID NOs: 4-488.

41-43. (canceled)

44. The engineered DNA polymerase of claim 1, having at least one improved property as compared to a reference DNA polymerase, wherein the improved property is selected from i) increased activity, ii) increased stability, iii) increased thermostability, iv) increased processivity, v) increased fidelity, vi) increased sensitivity to input target RNA or DNA, vii) increased product yield in an isothermal amplification reaction, viii) increased salt tolerance, and ix) increased resistance to an inhibitor, or any combinations of i), ii), iii), iv), v), vi), vii) and viii), wherein the reference DNA polymerase has an amino acid sequence corresponding to residues 12 to 604 of SEQ ID NO: 2, 10, 80, 224, or 366, or an amino acid sequence corresponding to SEQ ID NO: 2, 10, 80, 224, or 366.

45-51. (canceled)

52. A recombinant polynucleotide comprising a polynucleotide sequence encoding an engineered DNA polymerase of claim 1.

53-57. (canceled)

58. An expression vector comprising at least a recombinant polynucleotide of claim 52.

59. (canceled)

60. (canceled)

61. A host cell comprising an expression vector of claim 58.

62. (canceled)

63. (canceled)

64. A method of producing an engineered DNA polymerase polypeptide in a host cell comprising culturing a host cell of claim 61 under suitable culture conditions such that the DNA polymerase is produced.

65-71. (canceled)

72. A method of preparing a complementary DNA of a target nucleic acid, comprising contacting a target nucleic acid with a DNA polymerase of claim 1 in presence of substrates and target nucleic acid under conditions suitable for production of a complementary DNA to all or a portion of the target nucleic acid.

73. (canceled)

74. A method for detecting presence of a target nucleic acid, comprising contacting a sample suspected of containing a target nucleic acid with a DNA polymerase of claim 1 in presence of substrates under conditions suitable for DNA polymerase mediated production of a DNA complementary to all or a portion of the target nucleic acid, and detecting presence of the complementary DNA.

75-82. (canceled)
